SEQUENCE LISTING (75 sequences)

SEQ ID NO: 1 - protein, 950 aa, Artificial Sequence
  Ad5-HVR1-SpyTag Hexon protein sequence
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAGSGGSGAHIVMVDAYKPTKGSGGSGTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 2 - DNA, 2853 bp, Artificial Sequence
  Ad5-HVR1-SpyTag Hexon DNA Sequence
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctggcagcggaggatccggcgcccatatcgtgatggtggacgcctacaagcctaccaaaggctctggcggaagcggcactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataa

SEQ ID NO: 3 - protein, 973 aa, Artificial Sequence
  Ad5-HVR2-SpyTag Hexon Protein Sequence
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVGSGGSGAHIVMVDAYKPTKGSGGSGPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 4 - DNA, 2922 bp, Artificial Sequence
  Ad5-HVR2-SpyTag Hexon DNA Sequence
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcggcagcggaggatccggcgcccatatcgtgatggtggacgcctacaagcctaccaaaggctctggcggaagcggccctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataa

SEQ ID NO: 5 - protein, 964 aa, Artificial Sequence
  Ad5-HVR5-SpyTag Hexon Protein Sequence
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSGSGGSGAHIVMVDAYKPTKGSGGSGPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 6 - DNA, 2895 bp, Artificial Sequence
  Ad5-HVR5-SpyTag Hexon DNA Sequence
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaggcagcggaggatccggcgcccatatcgtgatggtggacgcctacaagcctaccaaaggctctggcggaagcggccctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataa

SEQ ID NO: 7 - protein, 960 aa, Artificial Sequence
  Ad5-HVR1-DogTag Hexon Protein Sequence
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAGSGGSGDIPATYEFTDGKHYITNEPIPPKGSGGSGTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 8 - DNA, 2883 bp, Artificial Sequence
  Ad5-HVR1-DogTag Hexon DNA Sequence
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctggcagcggaggatccggcgatattccggctacatatgaatttaccgatggtaaacattatatcaccaatgaaccgataccgccgaaaggctctggcggaagcggcactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataa

SEQ ID NO: 9 - protein, 983 aa, Artificial Sequence
  Ad5-HVR2-DogTag Hexon Protein Sequence
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVGSGGSGDIPATYEFTDGKHYITNEPIPPKGSGGSGPKYADKTFQPEPQIGESQWYETEINHAAGRVLKKTTPMKPCYGSYAKPTNENGGQGILVKQQNGKLESQVEMQFFSTTEATAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 10 - DNA, 2952 bp, Artificial Sequence
  Ad5-HVR2-DogTag Hexon DNA Sequence
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcggcagcggaggatccggcgatattccggctacatatgaatttaccgatggtaaacattatatcaccaatgaaccgataccgccgaaaggctctggcggaagcggccctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataa

SEQ ID NO: 11 - protein, 974 aa, Artificial Sequence
  Ad5-HVR5-DogTag Hexon Protein Sequence
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSGSGGSGDIPATYEFTDGKHYITNEPIPPKGSGGSGPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 12 - DNA, 2925 bp, Artificial Sequence
  Ad5-HVR5-DogTag Hexon DNA Sequence
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaggcagcggaggatccggcgatattccggctacatatgaatttaccgatggtaaacattatatcaccaatgaaccgataccgccgaaaggctctggcggaagcggccctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataa

SEQ ID NO: 13 - protein, 252 aa, Artificial Sequence
  pIX-(EAAAK3)-SpyCatcher Protein Sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKGSDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 14 - DNA, 759 bp, Artificial Sequence
  pIX-(EAAAK3)-SpyCatcher DNA Sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaagggatccgacagcgccacacacatcaagttcagcaagagagatgaggacggcaaagagctggctggcgcaacaatggaactgagagatagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacatttaa

SEQ ID NO: 15 - protein, 125 aa, Artificial Sequence
  DogCatcher Protein Sequence
MGSSHHHHHHSSGLVPRGSHMKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 16 - DNA, 378 bp, Artificial Sequence
  DogCatcher DNA Sequence
atgggcagcagccatcatcatcatcatcacagcagcggcctggtgccgcgcggcagccatatgaaactgggcgatattgaatttattaaagtgaacaaaaacgataaaaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcagtaa

SEQ ID NO: 17 - protein, 160 aa, Artificial Sequence
  DogCatcher-NANP9 Protein Sequence
MAHHHHHHVGTGKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQGSGGSGGSNANPNANPNANPNANPNANPNANPNANPNANPNANP

SEQ ID NO: 18 - DNA, 483 bp, Artificial Sequence
  DogCatcher-NANP9 DNA Sequence
atggcacatcaccaccaccatcacgtgggtaccggtaaactgggcgatattgaatttattaaagtgaacaaaaacgataaaaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcagggctctggcggaagcggcggatccaatgcgaaccctaatgcgaatcccaatgcaaatcccaatgcgaaccctaacgcaaatccgaacgcaaaccctaacgcgaaccctaatgctaatcctaacgccaatccttaa

SEQ ID NO: 19 - protein, 196 aa, Artificial Sequence
  DogCatcher-NANP18 Protein Sequence
MAHHHHHHVGTGKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQGSGGSGGSNANPNANPNANPNANPNANPNANPNANPNANPNANPNANPNANPNANPNANPNANPNANPNANPNANPNANP

SEQ ID NO: 20 - DNA, 591 bp, Artificial Sequence
  DogCatcher-NANP18 DNA Sequence
atggcacatcaccaccaccatcacgtgggtaccggtaaactgggcgatattgaatttattaaagtgaacaaaaacgataaaaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcagggctctggcggaagcggcggatccaatgctaaccctaacgctaaccccaacgccaatccgaatgcgaatcctaacgccaatccaaatgccaatccgaacgcgaacccaaacgctaatccaaacgcgaatccaaatgcgaaccctaatgcgaatcccaatgcaaatcccaatgcgaaccctaacgcaaatccgaacgcaaaccctaacgcgaaccctaatgctaatcctaacgccaatccttaa

SEQ ID NO: 21 - protein, 292 aa, Artificial Sequence
  DogCatcher-NANP Domain Protein Sequence
MAHHHHHHVGTGKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQGSGGSGGSNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANP

SEQ ID NO: 22 - DNA, 879 bp, Artificial Sequence
  DogCatcher-NANP Domain DNA Sequence
atggcacatcaccaccaccatcacgtgggtaccggtaaactgggcgatattgaatttattaaagtgaacaaaaacgataaaaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcagggctctggcggaagcggcggatccaatgcaaatccgaatgttgatccgaacgcgaacccgaacgtggaccctaacgccaatcctaatgtggacccaaatgcgaatccaaatgctaacccaaacgcaaacccgaatgcgaaccccaatgccaatccgaacgctaatcccaatgctaatcctaatgcaaatccaaacgcgaatccgaacgccaatcctaacgcaaacccgaacgcaaatccaaatgcaaacccaaatgctaatcctaatgcgaacccgaatgctaacccgaatgcaaaccctaacgttgaccctaatgctaaccctaacgctaaccccaacgccaatccgaatgcgaatcctaacgccaatccaaatgccaatccgaacgcgaacccaaacgctaatccaaacgcgaatccaaatgcgaaccctaatgcgaatcccaatgcaaatcccaatgcgaaccctaacgcaaatccgaacgcaaaccctaacgcgaaccctaatgctaatcctaacgccaatccttaa

SEQ ID NO: 23 - protein, 411 aa, Artificial Sequence
  SpyTag-MBP Protein Sequence
MGSSHHHHHHSSGLVPRGSHMGAHIVMVDAYKPTKGSGESGKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSS

SEQ ID NO: 24 - DNA, 1236 bp, Artificial Sequence
  SpyTag-MBP DNA Sequence
atgggcagcagccatcatcatcatcatcacagcagcggcctggtgccgcgcggcagccatatgggagcccacatcgtgatggtggacgcctacaagccgacgaagggtagtggtgaaagtggtaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgtaa

SEQ ID NO: 25 - protein, 121 aa, Artificial Sequence
  SnoopLigase Protein Sequence
MGSWSHHHHHHSSGGSGVNKNDKKPLRGAVFSLQKQHPDYPDIYGAIDQNGTYQNVRTGEDGKLTFKNLSDGKYRLFENSEPPGYKPVQNKPIVAFQIVNGEVRDVTSIVPPGVPATYEFT

SEQ ID NO: 26 - DNA, 366 bp, Artificial Sequence
  SnoopLigase DNA Sequence
atgggcagctggagccatcatcatcatcatcacagctctggtggtagtggtgtgaataagaacgataaaaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccccgggctataaaccggtgcagaataagccgattgtggcgtttcagattgtgaatggcgaagtgcgtgatgtgaccagcattgtgccgccgggtgtgccggctacatatgaatttacctaa

SEQ ID NO: 27 - protein, 109 aa, Artificial Sequence
  SnoopTagJr-AffiHER2 Protein Sequence
MGSSHHHHHHSSGGKLGSIEFIKVNKGSGESGSGASMTGGQQMGRDPGVDNKFNKEMRNAYWEIALLPNLNNQQKRAFIRSLYDDPSQSANLLAEAKKLNDAQAPKGLE

SEQ ID NO: 28 - DNA, 330 bp, Artificial Sequence
  SnoopTagJr-AffiHER2 DNA Sequence
atgggcagcagccatcatcatcatcatcacagcagcggcgggaaactgggctctattgaatttattaaagtgaacaaaggcagtggtgagtcgggatccggagctagcatgactggtggacagcaaatgggtcgggatccgggcgtggacaacaaattcaacaaagaaatgaggaacgcttactgggagatagctcttttacccaacttaaacaatcaacagaaaagggctttcataaggtcgttatacgatgacccaagccaaagcgctaaccttttagcagaagctaaaaagctaaatgatgctcaggcgccgaaaggcctcgagtaa

SEQ ID NO: 29 - protein, 139 aa, Artificial Sequence
  SpyCatcher Protein Sequence
MSYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 30 - DNA, 420 bp, Artificial Sequence
  SpyCatcher DNA Sequence
atgtcgtactaccatcaccatcaccatcacgattacgacatcccaacgaccgaaaacctgtattttcagggcgccatggttgataccttatcaggtttatcaagtgagcaaggtcagtccggtgatatgacaattgaagaagatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatttaa

SEQ ID NO: 31 - protein, 744 aa, Artificial Sequence
  HCMV gH-SpyTag Protein Sequence
MRPGLPSYLIILAVCLFSHLLSSRYGAEAVSEPLDKAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGEQPTTVPPPIDLSIPHVWMPPQTTPHGWTESHTTSGLHRPHFNQTCILFDGHDLLFSTVTPCLHQGFYLIDELRYVKITLTEDFFVVTVSIDDDTPMLLIFGHLPRVLFKAPYQRDNFILRQTEKHELLVLVKKDQLNRHSYLKDPDFLDAALDFNYLDLSALLRNSFHRYAVDVLKSGRCQMLDRRTVEMAFAYALALFAAARQEEAGAQVSVPRALDRQAALLQIQEFMITCLSQTPPRTTLLLYPTAVDLAKRALWTPNQITDITSLVRLVYILSKQNQQHLIPQWALRQIADFALKLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYIVTNQYLIKGISYPVSTTVVGQSLIITQTDSQTKCELTRNMHTTHSITVALNISLENCAFCQSALLEYDDTQGVINIMYMHDSDDVLFALDPYNEVVVSSPRTHYLMLLKNGTVLEVTDVVVDATDSRLLGSGGSGAHIVMVDAYKPTKHHHHHH

SEQ ID NO: 32 - DNA, 2235 bp, Artificial Sequence
  HCMV gH-SpyTag DNA Sequence
atgcggccaggcctcccctcctacctcatcatcctcgccgtctgtctcttcagccacctactttcgtcacgatatggcgcagaagccgtatccgaaccgctggacaaagcgtttcacctactgctcaacacctacgggagacccatccgcttcctgcgtgaaaataccacccagtgtacctacaacagcagcctccgtaacagcacggtcgtcagggaaaacgccatcagtttcaactttttccaaagctataatcaatactatgtattccatatgcctcgatgtctttttgcgggtcctctggcggagcagtttctgaaccaggtagatctgaccgaaaccctggaaagataccaacagagacttaacacttacgcgctggtatccaaagacctggccagctaccgatctttttcgcagcagctaaaggcacaagacagcctaggtgaacagcccaccactgtgccaccgcccattgacctgtcaatacctcacgtttggatgccaccgcaaaccactccacacggctggacagaatcacataccacctcaggactacaccgaccacactttaaccagacctgtatcctctttgatggacacgatctactattcagcaccgtcacaccttgtttgcaccaaggcttttacctcatcgacgaactacgttacgttaaaataacactgaccgaggacttcttcgtagttacggtgtccatagacgacgacacacccatgctgcttatcttcggccatcttccacgcgtacttttcaaagcgccctatcaacgcgacaactttatactacgacaaactgaaaaacacgagctcctggtgctagttaagaaagatcaactgaaccgtcactcttatctcaaagacccggactttcttgacgccgcacttgacttcaactacctagacctcagcgcactactacgtaacagctttcaccgttacgccgtggatgtactcaagagcggtcgatgtcagatgctggaccgccgcacggtagaaatggccttcgcctacgcattagcactgttcgcagcagcccgacaagaagaggccggcgcccaagtctccgtcccacgggccctagaccgccaggccgcactcttacaaatacaagaatttatgatcacatgcctctcacaaacaccaccacgcaccacgttgctgctgtatcccacggccgtggacctggccaaacgagccctttggacaccgaatcagatcaccgacatcaccagcctcgtacgcctggtctacatactctctaaacagaatcagcaacatctcatcccccaatgggcactacgacagatcgccgactttgccctaaaactacacaaaacgcacctggcctcttttctttcagccttcgcacgccaagaactctacctcatgggcagcctcgtccactccatgctggtacatacgacggagagacgcgaaatcttcatcgtagaaacgggcctctgttcattggccgagctatcacactttacgcagttgttagctcatccacaccacgaatacctcagcgacctgtacacaccctgttccagtagcgggcgacgcgatcactcgctcgaacgcctcacgcgtctcttccccgatgccaccgtccccgctaccgttcccgccgccctctccatcctatctaccatgcaaccaagcacgctggaaaccttccccgacctgttttgcttgccgctcggcgaatccttctccgcgctgaccgtctccgaacacgtcagttatatcgtaacaaaccagtacctgatcaaaggtatctcctaccctgtctccaccaccgtcgtaggccagagcctcatcatcacccagacggacagtcaaactaaatgcgaactgacgcgcaacatgcataccacacacagcatcacagtggcgctcaacatttcgctagaaaactgcgccttttgccaaagcgccctgctagaatacgacgacacgcaaggcgtcatcaacatcatgtacatgcacgactcggacgacgtccttttcgccctggatccctacaacgaagtggtggtctcatctccgcgaactcactacctcatgcttttgaaaaacggtacggtactagaagtaactgacgtcgtcgtggacgccaccgacagtcgtctcctcggaagcggaggctctggtgcccatatcgtgatggtggacgcctacaagcctaccaaacatcatcaccatcaccactaa

SEQ ID NO: 33 - protein, 278 aa, Artificial Sequence
  HCMV gL Protein Sequence
MCRRPDCGFSFSPGPVILLWCCLLLPIVSSAAVSVAPTAAEKVPAECPELTRRCLLGEVFEGDKYESWLRPLVNVTGRDGPLSQLIRYRPVTPEAANSVLLDEAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 34 - DNA, 837 bp, Artificial Sequence
  HCMV gL DNA Sequence
atgtgccgccgcccggattgcggcttctctttctcacctggaccggtgatactgctgtggtgttgccttctgctgcccattgtttcctcagccgccgtcagcgtcgctcctaccgccgccgagaaagtccccgcggagtgccccgaactaacgcgccgatgcttgttgggtgaggtgtttgagggtgacaagtatgaaagttggctgcgcccgttggtgaatgttaccgggcgcgatggcccgctatcgcaacttatccgttaccgtcccgttacgccggaggccgccaactccgtgctgttggacgaggctttcctggacactctggccctgctgtacaacaatccggatcaattgcgggccctgctgacgctgttgagctcggacacagcgccgcgctggatgacggtgatgcgcggctacagcgagtgcggcgatggctcgccggccgtgtacacgtgcgtggacgacctgtgccgcggctacgacctcacgcgactgtcatacgggcgcagcatcttcacggaacacgtgttaggcttcgagctggtgccaccgtctctctttaacgtggtggtggccatacgcaacgaagccacgcgtaccaaccgcgccgtgcgtctgcccgtgagcaccgctgccgcgcccgagggcatcacgctcttttacggcctgtacaacgcagtgaaggaattctgcctgcgtcaccagctggacccgccgctgctacgccacctagataaatactacgccggactgccgcccgagctgaagcagacgcgcgtcaacctgccggctcactcgcgctatggccctcaagcagtggatgctcgctaa

SEQ ID NO: 35 - protein, 129 aa, Artificial Sequence
  HCMV UL131A Protein Sequence
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 36 - DNA, 498 bp, Artificial Sequence
  HCMV UL131A DNA Sequence
atgcggctgtgtcgggtgtggctgtctgtttgtctgtgcgccgtggtgctgggtcagtgccagcgggaaaccgcggaaaaaaacgattattaccgagtaccgcattactgggacgcgtgctctcgcgcgctgcccgaccaaacccgttacaagtatgtggaacagctcgtggacctcacgttgaactaccactacgatgcgagccacggcttggacaactttgacgtgctcaagaggtgagggtacgcgctaaagatgcatgacaacgggaaggtaagggcgaacgggtaacgggtaagtaaccgcatggggtatgaaatgacgttcggaacctgtgcttgcagaatcaacgtgaccgaggtgtcgttgctcatcagcgactttagacgtcagaaccgtcgcggcggcaccaacaaaaggaccacgttcaacgccgccggttcgctggcgccacacgcccggagcctcgagttcagcgtgcggctctttgccaactag

SEQ ID NO: 37 - protein, 171 aa, Artificial Sequence
  HCMV UL128 Protein Sequence
MSPKDLTPFLTALWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 38 - DNA, 759 bp, Artificial Sequence
  HCMV UL128 DNA Sequence
atgagtcccaaagatctgacgccgttcttgacggcgttgtggctgctattgggtcacagccgcgtgccgcgggtgcgcgcagaagaatgttgcgaattcataaacgtcaaccacccgccggaacgctgttacgatttcaaaatgtgcaatcgcttcaccgtcgcgtacgtattttcatgattgtctgcgttctgtggtgcgtctggatctgtctctcgacgtttctgatagccatgttccatcgacgatcctcgggaatgccagagtagattttcatgaatccacaggctgcggtgtccggacggcgaagtctgctacagtcccgagaaaacggctgagattcgcgggatcgtcaccaccatgacccattcattgacacgccaggtcgtacacaacaaactgacgagctgcaactacaatccgtaagtctcttcctgagggccttacagcctatgggagagtaagacagagagggacaaaacatcattaaaaaaaaaagtctaatttcacgttttgtaccccccttcccctccgtgttgtaggttatacctcgaagctgacgggcgaatacgctgcggcaaagtaaacgacaaggcgcagtacctgctgggcgccgctggcagcgttccctatcgatggatcaatctggaatacgacaagataacccggatcgtgggcctggatcagtacctggagagcgttaagaaacacaaacggctggatgtgtgccgcgctaaaatgggctatatgctgcagtga

SEQ ID NO: 39 - protein, 233 aa, Artificial Sequence
  HCMV UL130 Protein Sequence
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIVGGGGSGGGGSGGGGSEPEA

SEQ ID NO: 40 - DNA, 702 bp, Artificial Sequence
  HCMV UL130 DNA Sequence
atgctgcggcttctgcttcgtcaccactttcactgcctgcttctgtgcgcggtttgggcaacgccctgtctggcgtctccgtggtcgacgctaacagcaaaccagaatccgtccccgccatggtctaaactgacgtattccaaaccgcatgacgcggcgacgttttactgtccttttctctatccctcgcccccacgatcccccttgcaattctcggggttccagcgggtatcaacgggtcccgagtgtcgcaacgagaccctgtatctgctgtacaaccgggaaggccagaccttggtggagagaagctccacctgggtgaaaaaggtgatctggtacctgagcggtcggaaccaaaccatcctccaacggatgccccgaacggcttcgaaaccgagcgacggaaacgtgcagatcagcgtggaagacgccaagatttttggagcgcacatggtgcccaagcagaccaagctgctacgcttcgtcgtcaacgatggcacacgttatcagatgtgtgtgatgaagctggagagctgggctcacgtcttccgggactacagcgtgtcttttcaggtgcgattgacgttcaccgaggccaataaccagacttacaccttctgcacccatcccaatctcatcgttggaggcggaggatctggcggaggtggaagtggcggaggcggatctgagcccgaggcctaa

SEQ ID NO: 41 - protein, 30 aa, Artificial Sequence
  SnoopTagJr-hTERT Protein Sequence
GKLGSIEFIKVNKGEARPALLTSRLRFIPK

SEQ ID NO: 42 - protein, 25 aa, Artificial Sequence
  SnoopTagJr-GGS-SIINFEKL (PEP1) Protein Sequence
GKLGSIEFIKVNKGGGSSIINFEKL

SEQ ID NO: 43 - protein, 25 aa, Artificial Sequence
  SnoopTagJr-AAY-SIINFEKL (PEP2) Protein Sequence
GKLGSIEFIKVNKGAAYSIINFEKL

SEQ ID NO: 44 - protein, 13 aa, Artificial Sequence
  Biotin-SnoopTagJr Protein Sequence
GKLGSIEFIKVNK

SEQ ID NO: 45 - protein, 18 aa, Artificial Sequence
  Biotin-SpyTag003 Protein Sequence
GSRGVPHIVMVDAYKRYK

SEQ ID NO: 46 - protein, 232 aa, Artificial Sequence
  Ad5-pIX-SpyCatcher (no linker) pIX sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 47 - DNA, 699 bp, Artificial Sequence
  Ad5-pIX-SpyCatcher (no linker) pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttgacagcgccacacacatcaagttcagcaagagagatgaggacggcaaagagctggctggcgcaacaatggaactgagagatagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacatttaa

SEQ ID NO: 48 - protein, 249 aa, Artificial Sequence
  Ad5-pIX-SpyCatcher (EAAAK3-GS linker) pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVEAAAKEAAAKEAAAKGSDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 49 - DNA, 750 bp, Artificial Sequence
  Ad5-pIX-SpyCatcher (EAAAK3-GS linker) pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttgaagccgccgctaaagaagctgctgccaaagaggccgctgcaaagggatccgacagcgccacacacatcaagttcagcaagagagatgaggacggcaaagagctggctggcgcaacaatggaactgagagatagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacatttaa

SEQ ID NO: 50 - protein, 250 aa, Artificial Sequence
  Ad5-pIX-SpyCatcher (GGS-EAAAK3 linker) pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 51 - DNA, 753 bp, Artificial Sequence
  Ad5-pIX-SpyCatcher (GGS-EAAAK3 linker) pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaaggacagcgccacacacatcaagttcagcaagagagatgaggacggcaaagagctggctggcgcaacaatggaactgagagatagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacatttaa

SEQ ID NO: 52 - protein, 247 aa, Artificial Sequence
  Ad5-pIX-SpyCatcher (EAAAK3 linker, no GGS or GS hinges) pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVEAAAKEAAAKEAAAKDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 53 - DNA, 744 bp, Artificial Sequence
  Ad5-pIX-SpyCatcher (EAAAK3 linker, no GGS or GS hinges) pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttgaagccgccgctaaagaagctgctgccaaagaggccgctgcaaaggacagcgccacacacatcaagttcagcaagagagatgaggacggcaaagagctggctggcgcaacaatggaactgagagatagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacatttaa

SEQ ID NO: 54 - protein, 262 aa, Artificial Sequence
  Ad5-pIX-SpyCatcher (GGS-EAAAK5-GS linker) pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKEAAAKEAAAKGSDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 55 - DNA, 789 bp, Artificial Sequence
  Ad5-pIX-SpyCatcher (GGS-EAAAK5-GS linker) pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaaggaggccgcagctaaggaagccgccgctaagggatccgacagcgccacacacatcaagttcagcaagagagatgaggacggcaaagagctggctggcgcaacaatggaactgagagatagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacatttaa

SEQ ID NO: 56 - protein, 243 aa, Artificial Sequence
  Ad5-pIX-SpyCatcher(deltaN1deltaC2) pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKGSDSATHIKFSKRDEDGKELAGATMELRDSSGKTISTWISDGQVKDFYLYPGKYTFVETAAPDGYEVATAITFTVNEQGQVTVNG

SEQ ID NO: 57 - DNA, 732 bp, Artificial Sequence
  Ad5-pIX-SpyCatcher(deltaN1deltaC2) pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaagggatccgacagcgccacccacatcaagttcagcaagagggacgaggacggcaaggagctggccggcgcaacaatggagctgagggacagcagcggcaagaccatcagcacctggatcagcgacggccaggtgaaggacttctacctgtaccccggcaagtacaccttcgtggagaccgccgcccccgacggctacgaggtggccaccgccatcaccttcaccgtgaacgagcagggccaggtgaccgtgaacggctaa

SEQ ID NO: 58 - protein, 274 aa, Artificial Sequence
  Ad5-pIX-SnoopCatcher pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 59 - DNA, 825 bp, Artificial Sequence
  Ad5-pIX-SnoopCatcher pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaagggatcccacatgaagcccctgaggggcgccgtgttcagcctgcagaagcagcaccccgactaccccgacatctacggcgccatcgaccagaacggcacctaccagaacgtgaggaccggcgaggacggcaagctgaccttcaagaacctgagcgacggcaagtacaggctgttcgagaacagcgagcccgccggctacaagcccgtgcagaacaagcccatcgtggccttccagatcgtgaacggcgaggtgagggacgtgaccagcatcgtgccccaggacatccccgccacctacgagttcaccaacggcaagcactacatcaccaacgagcccatcccccccaagtaa

SEQ ID NO: 60 - protein, 264 aa, Artificial Sequence
  Ad5-pIX-DogCatcher pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKGSKLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 61 - DNA, 795 bp, Artificial Sequence
  Ad5-pIX-DogCatcher pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaagggatccaaactgggcgatattgaatttattaaagtgaacaaaaacgataaaaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcagtaa

SEQ ID NO: 62 - protein, 173 aa, Artificial Sequence
  Ad5-pIX-SpyTag pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKGSAHIVMVDAYKPTK

SEQ ID NO: 63 - DNA, 522 bp, Artificial Sequence
  Ad5-pIX-SpyTag pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaagggatccgcccacatcgttatggtggatgcctacaagcccaccaaataa

SEQ ID NO: 64 - protein, 172 aa, Artificial Sequence
  Ad5-pIX-SnoopTagJr pIX protein sequence
MSTNSFDGSIVSSYLTTRMPPWAGVRQNVMGSSIDGRPVLPANSTTLTYETVSGTPLETAASAAASAAAATARGIVTDFAFLSPLASSAASRSSARDDKLTALLAQLDSLTRELNVVSQQLLDLRQQVSALKASSPPNAVGGSEAAAKEAAAKEAAAKGSKLGSIEFIKVNK

SEQ ID NO: 65 - DNA, 519 bp, Artificial Sequence
  Ad5-pIX-SnoopTagJr pIX DNA sequence
atgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggttggaggctccgaagccgccgctaaagaagctgctgccaaagaggccgctgcaaagggatccaaactgggctctattgaatttattaaagtgaacaaataa

SEQ ID NO: 66 - protein, 1311 aa, Artificial Sequence
  SARS CoV2 Spike-SnoopTagJr protein sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGIVQQQNNLLRAIEAQQHLLQLTVWGIKQLQARILAGGSGGHTTWMEWDREINNYTSLIHSLIEESQNQQEKNEQELLEGSGGSGKLGSIEFIKVNKEPEA

SEQ ID NO: 67 - DNA, 3936 bp, Artificial Sequence
  SARS CoV2 Spike-SnoopTagJr DNA sequence
atgttcgtgtttctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccctggcagcgcctcttctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctacaggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcaaggctctggaattgtgcagcagcaaaacaatctgctgcgggccatcgaggctcagcagcatctgctgcagctgaccgtgtggggaatcaagcagctccaggccagaatcctggctggtggatctggcggccataccacctggatggaatgggacagagagatcaacaactacaccagcctgatccacagcctgattgaggaatcccagaatcagcaagagaagaacgagcaagaactgctggaaggaagcggaggctctggtaaactgggctctattgaatttattaaagtgaacaaagagcccgaagcctaa

SEQ ID NO: 68 - protein, 251 aa, Artificial Sequence
  SARS CoV2 Spike RBD-SnoopTagJr protein sequence
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSGGSGKLGSIEFIKVNKEPEA

SEQ ID NO: 69 - DNA, 756 bp, Artificial Sequence
  SARS CoV2 Spike RBD-SnoopTagJr DNA sequence
atgttcgtgtttctggtgctgctgcctctggtgtccagccagtgtcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatggaagcggaggctctggtaaactgggctctattgaatttattaaagtgaacaaagagcccgaagcctaa

SEQ ID NO: 70 - protein, 359 aa, Artificial Sequence
  SARS CoV2 Spike RBD-SnoopCatcher protein sequence
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSGGSGGSGGSGHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPKEPEA

SEQ ID NO: 71 - DNA, 1080 bp, Artificial Sequence
  SARS CoV2 Spike RBD-SnoopCatcher DNA sequence
atgttcgtgtttctggtgctgctgcctctggtgtccagccagtgtcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatggaagcggaggctctggtggatccggtggatctggacacatgaagcctctgagaggcgccgtgttcagcctgcagaaacagcaccctgactaccccgatatctacggcgccatcgaccagaacggcacctaccagaatgttcggacaggcgaggatggcaagctgaccttcaagaacctgagcgacggcaagtaccggctgttcgagaattctgagcctgccggctacaagcccgtgcagaacaaacctatcgtggccttccagatcgtgaacggcgaagtgcgggatgtgaccagcatcgtgcctcaggatatccccgccacctacgagttcaccaacggcaagcactacatcaccaacgagcccattcctccaaaagagcccgaagcctaa

SEQ ID NO: 72 - protein, 362 aa, Artificial Sequence
  SnoopCatcher-SARS CoV2 Spike RBD protein sequence
METDTLLLWVLLLWVPGSTGDGHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPKGSGGSGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNEPEA

SEQ ID NO: 73 - DNA, 1089 bp, Artificial Sequence
  SnoopCatcher-SARS CoV2 Spike RBD DNA sequence
atggagacagacacactcctgctatgggtactgctgctctgggttccaggttccactggtgacggtcacatgaagcccctgaggggcgccgtgttcagcctgcagaagcagcaccccgactaccccgacatctacggcgccatcgaccagaacggcacctaccagaacgtgaggaccggcgaggacggcaagctgaccttcaagaacctgagcgacggcaagtacaggctgttcgagaacagcgagcccgccggctacaagcccgtgcagaacaagcccatcgtggccttccagatcgtgaacggcgaggtgagggacgtgaccagcatcgtgccccaggacatccccgccacctacgagttcaccaacggcaagcactacatcaccaacgagcccatcccccccaagggctctggcggaagcggcggatcccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatgagcccgaggcctaa

SEQ ID NO: 74 - protein, 337 aa, Artificial Sequence
  SARS CoV2 Spike RBD-SpyCatcher protein sequence
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNGSGGSGGSGGSGDSATHIKFSKRDEDGKELAGATMELRDSSGKTISTWISDGQVKDFYLYPGKYTFVETAAPDGYEVATAITFTVNEQGQVTVNGKATKGDAHIEPEA

SEQ ID NO: 75 - DNA, 1014 bp, Artificial Sequence
  SARS CoV2 Spike RBD-SpyCatcher DNA sequence
atgttcgtgtttctggtgctgctgcctctggtgtccagccagtgtcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatggaagcggaggctctggtggatccggtggatctggcgattctgccacacacatcaagttcagcaagcgcgacgaggacggcaaagaactggctggcgctaccatggaactgagagacagcagcggcaagaccatcagcacctggatctccgacggccaagtgaaggacttctatctgtaccccggcaagtacaccttcgtggaaaccgccgctcctgacggatatgaagtggccaccgccatcaccttcaccgtgaatgagcagggacaagtgaccgtgaacggcaaggccacaaaaggcgacgcccacattgagcccgaagcctaa

(19) United States
(12) Patent Application Publication
(10) Pub. No.: US 2022/0372514 A1
(43) Pub. Date: Nov. 24, 2022

(54) VIRUSES WITH MODIFIED CAPSID PROTEINS

(71) Applicant: SpyBiotech Limited, Oxford (GB)

(72) Inventors: Matthew DICKS, Oxford (GB); Mark HOWARTH, Oxford (GB); Sumi BISWAS, Oxford (GB)

(21) Appl. No.: 17/755,447

(22) PCT Filed: Nov. 2, 2020
(86) PCT No.: PCT/GB2020/052774

§ 371 Date: Apr. 29, 2022

(30) Foreign Application Priority Data
Nov. 1, 2019 (GB) 1915905.2

(51) Int. Cl. C12N 15/86; A61K 35/761

(52) U.S. Cl. CPC C12N 15/86; A61K 35/761; C12N 2710/10322; C12N 2710/10343; C12N 2710/10334; C12N 2710/10352; C12N 2710/10332; C12N 2710/10345; A61K 39/00

(57) ABSTRACT

This invention relates to preparations comprising adenoviral vectors with modified capsid proteins. These modified capsid proteins enable customisable decoration of the adenoviral vector to be performed, enabling diverse applications from personalised cancer vaccines to targeted gene therapy vectors, and mixtures of the same. In particular, the adenoviral vectors with modified capsid proteins may be modified in the hexon and/or pIX capsid proteins. The invention makes use of peptide pairs to provide a “primed” adenovirus which is ready for decoration.

### FIELD OF THE INVENTION

This invention relates to preparations comprising adenoviral vectors with modified capsid proteins. These modified capsid proteins enable customisable decoration of the adenoviral vector to be performed, enabling diverse applications from personalised cancer vaccines to targeted gene therapy vectors, and mixtures of the same.

In particular, the adenoviral vectors with modified capsid proteins may be modified in the hexon and/or pIX capsid proteins.

Furthermore, the adenoviral vectors of the present invention are less susceptible to clearance by neutralising antibodies than the vectors of the prior art. Advantageously, therefore, these modified adenoviral vectors may evade the host immune response.

A preferred aspect of the invention is to use adenoviral vectors with modified capsid proteins in the preparation of vaccines. These vaccines may be prophylactic or therapeutic. In this aspect of the invention, the modified capsid proteins enable modular covalent display of antigens, thereby inducing immune responses to said antigens, and compositions comprising the same. In particular it relates to an adenoviral vector with modified capsid proteins that permits the quick assembly of personalised vaccine therapies, in order to fight disease. The inventors have shown that the display of antigens using adenoviral vectors increases the immune response to the antigen.

Other aspects of the invention include the use of the adenoviral vectors with modified capsid proteins in the preparation of oncolytic viruses, gene therapy vectors and/or retargeting of adenoviral tropism.

Yet further aspects of the invention allow the combination of different types of modification to enable several therapeutic applications simultaneously, such as an oncolytic virus expressing cancer antigens.

### BACKGROUND OF THE INVENTION

Adenoviruses (Ads) have an icosahedral protein capsid that surrounds the linear duplex genome. No lipid envelope is present. The capsid includes the structural proteins hexon, fiber, penton, IIIa, VIII and IX. It is thought that the fiber capsid protein aids attachment to the host cell, via the knob domain. Ads rely upon host infection in order to be able to replicate using the host cell's replication machinery. There are at least 57 serotypes of human adenovirus, Ads1-57 that may be grouped into seven “species” A-G. Similarly, animal Ads exist, such as canine and equine Ads, also classifiable into various serotypes and “species”. Serotypes are generally defined by the ability of antisera to neutralise the infection of cells in vitro. These viruses are well studied and understood, they can be grown in high titres, they can infect both dividing and non-dividing cells and can be maintained in host cells as an episome. These characteristics make them a good therapeutic choice, since nearly all trials have shown they are safe and well-tolerated.

The icosahedral capsid is made up of several proteins. Hexon is the major protein forming the 20 triangular faces of the viral capsid. The hexon proteins form trimers, and each trimer interacts with six other trimers. The 12 vertices are formed by the penton capsomere, these are a complex of 3 fiber proteins and five penton proteins. A long fiber extends from each vertex, composed of three identical chains that form a knob at the end. The capsid also includes minor proteins, notably amongst them pIIIa, pVI, pVIII and pIX. These minor capsid proteins may be located on the inner or outer surface of the capsid and may have additional functions beyond structural ones. Exemplarily, pVI may facilitate nuclear import of hexon proteins and pIX may be involved with DNA packaging into the capsid and transcriptional activation.

Ads are commonly used for gene therapy, in particular as gene delivery vectors, due to their capacity for inclusion of additional genetic sequences. Over 2,000 gene therapy trials have been conducted using Ads. Adenoviral vectors allow for the transmission of the transgene they carry into the host nucleus but do not integrate viral DNA into the host chromosome. The insert size for Ads when used as gene therapy vectors is large, with a capacity of 8-36 kb possible.

Oncolytic adenoviruses are being explored for use in cancer, most notably for the treatment of head and neck cancer. Oncolytic viruses preferentially infect and kill cancer cells, the process of oncolysis releasing new infective adenoviral virions and also recruiting the host immune system to raise an anti-cancer response. Various means have been used to ensure the targeting of the oncolytic adenovirus to the cancer cell, including the use of adapter molecules such as fusion proteins including an antibody to a capsid protein, directly modifying the viral capsid proteins or using transcriptional targeting.

Additionally, Ads have emerged as a promising vaccine delivery vehicle due to their ability to induce both innate and adaptive immune responses; having the capacity to induce potent antigen-specific B and T cell immune responses. Adenoviral vectors are highly immunogenic and are efficient in delivering antigens. However, this potential has yet to be fully realised. Several adenoviral vector based vaccine candidates were developed and pursued further in clinical trials, however, these were not successful. Adenovirus of the human serotype 5 (AdHu5) based vaccine, developed against HIV-1 by Merck, induced CD8⁺ T cell responses but failed to prevent HIV infections.

A major obstacle to the success of adenoviral based vectors in human and animal therapy is the neutralisation by virus-specific antibodies. Natural infection by adenovirus is high in human and animal populations, and therefore the adaptive immune system may recognise and respond to the presence of adenoviral vectors by the secretion of neutralising antibody (NAB). Similarly, the innate immune system may also be responsible for assisting the response to adenoviral vectors. It is estimated that 50% to 90% of the adult population has pre-existing immunity to AdHu5 for example. Such a response can clear the therapeutic adenoviral vectors before the desired effect is seen. Solving this issue would enable adenoviral therapeutic vectors to be more routinely used.

Various approaches have been taken to evade the host immune response in the use of adenoviral vectors. One approach was to swap the hypervariable regions (HVR) of AdHu5 with hypervariable regions from another adenoviral serotype, in order to produce a “chimera”. This may allow the chimera to avoid AdHu5 neutralising antibodies, but will only be effective in the absence of neutralising antibodies against the alternative serotype. An improved means of evading the immune response is desirable.

Ads have been used as vaccines, mainly as DNA vaccine delivery vectors by including within the adenoviral genome the genetic sequence for an antigen that is expressed within the host cell. Alternative ways of using Ads as vaccine compositions include the use of the adenovirus particle itself to display antigens. This approach has had varying degrees of success, depending on the nature of the decoration, such as the use of genetic fusions, use of modified antibodies to bind to capsid proteins and click chemistry. Incorporating immunogenic peptides into the capsid offers potential advantages, such as the ability to induce a strong humoral response, similar to the response generated by native capsid proteins.

One approach is to modify the adenoviral capsid proteins to include an immunogenic peptide for display on the virus surface. A major obstacle to the “antigen capsid-incorporation” strategy is the limitation this places on the size of insertion, since it must not disrupt the natural folding of the capsid protein, nor affect the interaction which holds the capsid proteins together. Further, one disadvantage of directly encoding the antigen within the capsid protein is the amount of time and work that is required to optimise the vaccine for each individual antigen. It would be desirable to formulate an adenovirus vector with a modified capsid protein that was ready for the attachment of any desired antigen, without the need for optimisation of the insertion within a capsid protein. Such a “primed” adenoviral vaccine composition would be of particular use when it comes to the preparation of personalised vaccines, where a vaccine is prepared in a bespoke manner for just one individual. The latter may be the case for personalised cancer vaccines or to raise an immune response to a particular drug-resistant microbiological infection and the like.

Indeed, given the current challenges with exploiting adenovirus as a therapeutic delivery vehicle due to the neutralisation of the virus, and an inability to include a sequence of longer than 100 amino acids into some of the capsid proteins including the most abundant capsid protein, hexon, there remains a pressing need to develop an approach which would allow for the utilisation of the clear benefits of an adenovirus vector, allow for a personalisation of the therapeutic and also reduce the immune clearance of the vector. It would be desirable to prepare a “primed” adenoviral vector, which has a modified capsid protein that is ready to accept decoration by an entity, such as an antigen, immunogenic peptide, protein, glycoprotein, antibody, targeting molecule, cell surface marker, protein, peptide, glycoprotein, lipoprotein and the like. The production of a “primed” adenovirus may permit the preparation of personalised therapeutics without laborious trial and error, since the modification of the capsid protein has already been completed and this reduces the restriction on the decoration of the viral capsid that may be achieved.

Adenoviral capsid proteins have been previously modified; most commonly the work is to alter the natural tropism of the viral vector, rather than to overcome the neutralisation of the virus-based vector. Altering the tropism, the particular cells and/or tissues of a host that support growth of the virus, is undertaken such that the viral vector may be targeted to a particular cell type for a particular indication. For example, a gene therapy adenoviral vector may wish to be directed only to retinal cells, and the tropism may be altered accordingly. For vaccine applications in particular, the natural tropism of the virus may be less important, since it is the aim of the viral vaccine vector to induce an immune response to the antigen it is displaying, rather than to infect any particular cell type.

The present invention concerns incorporating modifications, such as peptide insertions, into the adenoviral capsid proteins in order to prepare a vector ready for addition of decoration. These insertions allow for modular assembly and display of a foreign entity on the surface of the adenovirus that are not limited in size. It also allows for the preparation of bespoke therapies, such as vaccines, for example personalised cancer vaccines or oncolytic viruses, with the minimal amount of preparation. The viral vector described here is a flexible platform, allowing the preparation of a multitude of different therapeutics using the same vector. This is a novel technology that has been demonstrated for the first time, using modular covalently-bonded display of multiple peptide and protein partners on the surface of the virus. Adenoviral vectors have not been successfully modified previously using the technology proposed by the current inventors.

The present invention provides a significant improvement over the art. The present inventors have shown that the technology enables the attachment of entities to the capsid proteins through the protein partner pairs that effectively shield the adenovirus from the effect of neutralising antibodies. This improvement is demonstrated here with the attachment of antigens, but in practice any entity could be attached in order to have this shielding or blocking effect. Notably, the inventors are particularly pleased to note that despite the large size of some of the entities added using the technology, the infectivity of the adenovirus is retained. Therefore, the present technology could be used to improve current gene therapy vectors or oncolytic adenovirus vectors by simply modifying a capsid protein and adding a shielding entity.

In relation to vaccine preparations, the present invention enables immune responses to be generated against displayed antigens. Advantageously, for the first time, the inventors demonstrate that the surface of the virus can be decorated with larger antigenic proteins that have an advantageous effect of blocking the neutralisation of the virus by potent neutralising monoclonal antibody. Thus, not only can the viral vaccine vector be used for a variety of antigen types, should the antigen be sufficiently large, the displayed antigens may also protect the viral vaccine vector from neutralisation by host antibody. This would ensure that the viral vaccine vector can persist for longer to induce an immune response, and could also enable the same vector to be used for multiple immunizations without reduction in efficacy associated with vector neutralisation.

Use of peptide binding pairs, such as SpyCatcher and SpyTag (WO2011/098772), based upon attachment proteins from a bacterium, has been established as a technology to irreversibly conjugate recombinant proteins and the like. Bio-conjugation between entities that would be impossible to achieve through genetic fusion between proteins can work using peptide binding pairs. Various Catcher and Tag pairs are now available, some based upon modifications of SpyCatcher and SpyTag and others based upon similar chemistry from alternative bacterial proteins.

In relation to adenovirus, the present inventors found that the inclusion of the peptide pairs within the adenoviral capsid proteins were not as routine or straightforward as postulated, particularly in relation to the provision of an attachment means for antigens. Indeed, work by the inventors has shown that the most commonly utilised peptide pair, in this case insertion of SpyTag into the hexon capsid protein did not allow for the formation of a functional adenoviral vector (a vector allowing for effective coupling of SpyCatcher and capable of infecting cells). Indeed, the data showing that SpyTag was poorly reactive after insertion is included in the Examples, together with data on the lack of infectivity of the modified adenovirus. Thus, significant work has been expended in developing a system that works in relation to a modified adenoviral vector that is ready to accept peptide pairing and attachment of an entity to the modified capsid protein via the peptide partner inserted into the capsid protein.

### SUMMARY OF THE INVENTION

In accordance with a first aspect of the invention, there is provided an adenoviral vector for preparation of a prophylactic or therapeutic adenoviral composition, comprising at least one modification in the capsid protein, wherein said modification comprises the inclusion of a first peptide partner in a capsid protein, wherein said first peptide is capable of forming a covalent bond with a second peptide partner, which can be attached to an entity.

The modification may be a fusion with the first peptide partner or an insertion of the first peptide partner into the capsid protein.

The capsid protein may be any capsid protein, but is preferably a hexon protein or a pIX protein.

The first peptide partner and the second peptide partner form a peptide partner pair, which may be covalently linked by an isopeptide or ester bond, preferably an isopeptide bond.

In one aspect, the first peptide partner is the “tag” partner, which may be covalently linked by an isopeptide or ester bond to a second peptide partner which is a “catcher”. In this aspect, the capsid protein is preferably a hexon protein. Thus, the first peptide partner or “tag” which modifies the hexon is preferably a DogTag, Isopeptag, Isopeptag-N, SdyTag, PsCsTag or Jo. It is preferred that the first peptide partner or tag is not SpyTag. In this embodiment, SpyTag is unmodified during its insertion into the hexon protein.

In an alternative aspect, the first peptide partner is the “catcher” partner, which may be covalently linked by an isopeptide or ester bond to a second peptide partner which is a “tag”. In this aspect, the capsid protein is preferably a hexon protein. Thus, the first peptide partner or “catcher” which modifies the hexon may be a DogCatcher, SpyCatcher, SnoopCatcher, Pilin-C, Pilin-N, SdyCatcher, PsCsCatcher or In.

In one aspect, the first peptide partner is the “catcher” partner, which may be covalently linked by an isopeptide or ester bond to a second peptide partner which is a “tag”. In this aspect, the capsid protein is preferably a pIX protein. Thus, the first peptide partner or “catcher” which modifies the pIX is preferably a SpyCatcher, DogCatcher, SnoopCatcher, Pilin-C, Pilin-N, SdyCatcher, PsCsCatcher or In.

In an alternative aspect, the first peptide partner is the “tag” partner, which may be covalently linked by an isopeptide or ester bond to a second peptide partner which is a “catcher”. In this aspect, the capsid protein is preferably a pIX protein. Thus, the first peptide partner or “tag” which modifies the pIX protein is preferably a SpyTag, SnoopTagJr, DogTag, Isopeptag, Isopeptag-N, SdyTag, PsCsTag or Jo.

In one aspect, the first peptide partner may be inserted into a hexon protein, and optionally the insertion into the hexon protein may be up to 200, up to 150 or up to 100 amino acids in length. The insertion into the hexon protein may be at any appropriate point, optionally in any one or more of the hypervariable HVR loops. The first peptide partner inserted into the hexon protein may be a DogTag. DogTag is capable of forming a spontaneous covalent bond with DogCatcher, or a covalent bond with SnoopTagJr or SnoopTag in a reaction requiring a catalyst, SnoopLigase. DogCatcher or SnoopTagJr or SnoopTag may therefore be the second peptide partner. DogTag or SnoopTagJr may therefore be the second peptide partner. The second peptide partner is linked or attached to an entity such as an antigen. It is surprising to the inventors that DogTag was able to be inserted into the hexon capsid protein to form a functional adenovirus vector for capsid display of protein partners after the failure of the SpyTag insertion, as described above. As the Examples show, the insertion of DogTag into the hexon and pairing with SnoopTagJr or DogCatcher enables the modified adenovirus to retain its infective ability in the cells tested after coupling. A wide range of SnoopTagJr and DogCatcher fusion proteins, some over 50 kDa in size, have been coupled efficiently and displayed on adenovirus with no reduction in viral infectivity. The retention of infectivity is important if the therapeutic use of the adenoviral vector requires cell entry, for example for gene therapy or as an oncolytic virus.

Other first peptide partners that may be included within the hexon protein that are less than 100 amino acids in length include:

Isopeptag which pairs with Pilin-C

Isopeptag-N which pairs with Pilin-N

SdyTag which pairs with SdyCatcher

PsCsTag which pairs with PsCsCatcher

Jo which pairs with In, or

RrgATag/RrgATag2/DogTag which pair with RrgACatcher (also denoted here as “DogCatcher”).

It is preferred that of the possible peptide partners that are included within the hexon protein, the inclusion is not an insertion of SpyTag. In this embodiment, SpyTag was inserted within various HVRs of hexon and found to reduce infectivity to an unsatisfactory level once paired with SpyCatcher. Thus, viral fitness was impaired.

In another aspect, the first peptide partner may be fused to the pIX capsid protein, optionally at the N- or C-terminal end, preferably at the C-terminal end. The first peptide partner fused to the pIX capsid protein may be a SpyCatcher, SnoopCatcher or DogCatcher. SpyCatcher is capable of forming a covalent bond with SpyTag, which herein forms the second peptide partner, and can therefore be attached to an antigen. SnoopCatcher is capable of forming a covalent bond with either SnoopTag or SnoopTagJr, and DogCatcher is capable of forming a covalent bond with DogTag, and may be used as a binding pair in either orientation as first or second peptide partner. The first peptide partner may also be a DogTag, SpyTag, SnoopTagJr or SnoopTag, wherein the matching second peptide partner is DogCatcher, SnoopTagJr, SnoopTag, SpyCatcher, DogTag, or SnoopCatcher. Other peptide partner pairs that may be suitable for fusion with pIX in either orientation are: RrgATag/RrgATag2/DogTag and RrgACatcher, Isopeptag/Pilin-C, Isopeptag-N/Pilin-N, SdyTag/SdyCatcher, PsCsTag/PsCsCatcher and Jo/In.

The insertion of SpyCatcher into pIX has been demonstrated to permit the coupling of SpyTag conjugated entities, including the HCMV pentamer, whilst retaining infectivity.

Particularly preferred may be the insertion of SnoopCatcher or DogCatcher into pIX, since these have both been demonstrated herein to have good adenoviral viability. These insertions are furthermore genetically stable for greater than 3 passages.

As used herein for any aspect of the invention, the first peptide partner is part of a pair of peptides that are capable of forming a covalent bond, such as an isopeptide bond or ester bond under the appropriate conditions. These are also known as protein tag and catcher pairs or protein tag and binding partner pairs. The first peptide partner may be a first peptide tag or may be a first peptide catcher. Each partner pair may comprise a tag and a catcher. The covalent bond that is formed may spontaneously react, or require the assistance of a third entity such as a ligase. Further information on suitable peptide pairs is included below.

Said adenoviral vector may be used in the preparation of a vaccine. The vaccine may be prophylactic or therapeutic. The invention therefore extends to a method of preparing a vaccine, comprising the use of an adenoviral vector as described herein. The method comprises the attachment of an antigen to the adenoviral vector via a second peptide partner. Said second peptide partner is attached to the antigen, preferably fused to said antigen, and is capable of forming a covalent bond with the first peptide partner present on the immunogenic adenoviral vector. The covalent bond, and therefore attachment, may occur spontaneously, or may require the use of a third entity to facilitate binding, such as a ligase. Thus, the antigen is attached to the adenovirus by means of the peptide partner pair, the first partner of which is included within a modified capsid protein.

In another aspect of the invention, there is provided a vaccine composition comprising an adenoviral vector including at least one modification in a capsid protein, wherein said modification comprises the inclusion of a first peptide partner, and said first peptide partner is covalently bonded to a second peptide partner attached to an antigen. Said adenoviral vector is as described extensively herein.

In another aspect of the invention, there is provided an immunogenic adenoviral vector comprising of at least one modification in the hexon capsid protein, wherein said modification comprises:

a. a first peptide partner; and
b. a second peptide partner attached to an antigen
wherein the first peptide partner and second peptide partner are coupled via a covalent bond.

In one embodiment of this aspect of the invention, the first peptide partner inserted into the hexon capsid protein is less than 200 amino acids in length, less than 150 amino acids in length, less than 100 amino acids in length, optionally wherein said first peptide partner is a DogTag. If DogTag is the first peptide partner, the second peptide partner may be DogCatcher or SnoopTagJr or SnoopTag. In order to assist the formation of a covalent bond between DogTag and SnoopTagJr or SnoopTag, a ligase may be utilised.

The first peptide partner inserted into the hexon may also be any one of SnoopTag, SnoopTagJr, SnoopCatcher, DogCatcher, Isopeptag, Pilin-C, Isopeptag-N, Pilin-N, SdyTag, SdyCatcher, PsCsTag, PsCsCatcher, RrgATag/RrgATag2, RrgACatcher, Jo, or In.

It is preferred that SpyTag is not used as the first peptide partner in the hexon protein, with SpyCatcher as the second peptide partner.

In another aspect of the invention, there is provided an immunogenic adenoviral vector comprising of at least one modification in the pIX capsid protein, wherein said modification comprises:
a. a first peptide partner; and
b. a second peptide partner attached to an antigen
wherein the first peptide partner and second peptide partner are coupled via a covalent bond.

According to one aspect, the first peptide partner may be a SpyCatcher protein. In this instance, the second peptide partner may be a SpyTag. The covalent bond that forms between these partner pairs is spontaneous and does not require assistance. SnoopCatcher or DogCatcher could equally be utilised as the first peptide partner, with their partner pairs being SnoopTag/SnoopTagJr or DogTag respectively. SnoopTagJr/SnoopTag or SpyTag could equally be utilised as the first peptide partner, with their partner pairs being SnoopCatcher or SpyCatcher respectively.

Equally, any one of the following pairs could be utilised: Isopeptag which pairs with Pilin-C
Isopeptag-N which pairs with Pilin-N
SdyTag which pairs with SdyCatcher
PsCsTag which pairs with PsCsCatcher
Jo which pairs with In, or
RrgATag/RrgATag2/DogTag which pair with RrgACatcher (DogCatcher).

Any of the modifications to the adenoviral capsid proteins with a first peptide partner described herein permits the construction of an adenoviral vector, which is effectively an adenoviral vector which has been “primed” for the addition of an entity in order to manufacture a prophylactic or therapeutic composition, such as the addition of an antigen to manufacture a vaccine. Alternatively, the adenoviral vector is primed to receive the attachment of a shielding entity whose sole purpose is to block the recognition and binding of host antibodies to the adenovirus.

The entity can then be added by contacting the adenoviral vector with the second peptide partner attached to the entity, which is capable of forming a spontaneous or assisted covalent bond with the first peptide partner on the adenoviral vector. Any entity can also be added using this technology. Thus, targeting entities or moieties, such as antibodies or fragments thereof, cell surface marker binding agents or partner pairs may be used. Alternatively, the entity may be a shielding entity. These may be a benign, unreactive or innocuous protein, polypeptide, peptide, glycopeptide, lipopeptide, polysaccharide or lipid whose sole function is to shield the adenovirus capsid from binding of host immune cells. In this context benign means an entity that has no additional function, such as an antigen for a vaccine or a targeting moiety. All the shielding entity does is provide a physical shield to the capsid that does not allow the binding of host antibodies. Nevertheless, the inventors have shown that it is possible to retain infectivity with such attachments, which is a surprise. This effect was particularly seen with modifications to the hexon, the major component of the viral capsid.

The adenoviral vector may be decorated by attaching more than one entity. The adenoviral vector may have attached a multiplicity of entities. Each entity may have a distinct function, for example, an antigen and a targeting moiety. Multiple entities may be attached, each one to a separate second peptide partner. This enables the attachment of multiple entities to a single adenoviral vector. The different entities may be for the same indication (for example different antigens for the same disease) or different indications (for example a combined vaccine composition for different diseases such as measles, mumps and rubella on a single adenoviral vector).

It is not generally an object of the invention in relation to vaccines to be altering the natural tropism of the adenovirus, since the present invention is seeking to raise antibodies or T cells to the presented antigen, rather than to retarget the vector to specific cell types. The fiber component is thought to be the major determinant of tropism. The inventors have seen for several Examples using a DogTag modification in the hexon protein that the modification to the capsid protein has little or no effect on the capability of transducing a specific target cell. Indeed, in the Examples, there was no drop in infectivity in HEK293 cells when DogTag was inserted into the hexon and paired with DogCatcher on the antigen. However, in some instances, the inventors have shown that modifications can affect the ability of the virus to infect HEK293 cells, herein this was demonstrated with SpyTag when inserted into the hexon and paired with SpyCatcher on the antigen. A 100-fold decrease in infectivity was seen for the SpyTag-inserted adenovirus. This is undesirable and unexpected. Indeed, given that this is the most commonly used peptide tag, and is frequently preferred for most applications, the inventors were of the opinion that adenovirus may be intransigent to the modification of the capsid protein using partner pairs. It was only through significant work that it was appreciated that this did not hold true for all partner pairs, and that DogTag in particular was a workable modification of the hexon protein.

The modified adenoviral vectors described here have been carefully engineered such that they are readily produced with yields comparable to vectors with a wild-type capsid protein, e.g. wild-type hexon or wild-type pIX capsid proteins. Methods of producing adenovirus in vitro are well known to those skilled in the art, along with methods of introducing recombinant genes into the adenovirus.

It is widely appreciated in the field that performing modifications and/or insertions to viral capsid proteins is difficult and often disruptive to viral function and replication. Modification must not inhibit correct folding of the capsid protein or its incorporation within the capsid structure of the virion during assembly. For antigen display in particular, modifications must be performed such that the conformation and location of the displayed antigen enables recognition by the immune system, and in the case of infectious viral particles, does not impede the ability of the viral vector to infect cells. Previous studies have demonstrated the complexity of these requirements. In the context of hexon HVR loops, Matthews et al showed that vector viability is dependent upon the size of insertion and which HVR loop is targeted. [Matthews Q L, et al Optimization of capsid-incorporated antigens for a novel adenovirus vaccine approach. Virol J. 2008 Aug. 21; 5:98. doi: 10.1186]. The same study also demonstrated that immune recognition by displayed epitopes was also dependent on the nature and location of the inserted antigen. Other studies, such as in Gu et al, have revealed the importance of the nature and length of linker sequences to facilitate efficient epitope display. [Linlin Gu, et al, A recombinant adenovirus-based vector elicits a specific humoral immune response against the V3 loop of HIV-1 gp120 in mice through the “Antigen Capsid-Incorporation” strategy, Virol J. 2014 Jun. 16; 11:112. doi: 10.1186]. In the present application, the present inventors again demonstrate this phenomenon, by showing that coupling of SpyCatcher to Ad5-HVR-SpyTag vectors is less efficient, and substantially impedes vector infectivity (FIG. 2), while coupling of Ad5-HVR-DogTag to DogCatcher is highly efficient and does not impede vector infectivity (FIG. 3). The superior efficiency and utility of DogTag:DogCatcher coupling compared to SpyTag:SpyCatcher coupling in the context of adenovirus hexon insertion was unexpected, since SpyTag:SpyCatcher coupling has been shown to be highly efficient in a wide variety of other contexts. Indeed, there are over 500 documented constructs using the SpyCatcher:SpyTag technology.

In the context of pIX modification, studies have demonstrated that the pIX protein can tolerate a variety of different extensions to the C-terminus. However, the viability, capsid stability, and efficiency of antigen display are highly dependent on the nature and length of the fusion [Igor P. Dmitriev, et al, Engineering of Adenovirus Vectors Containing Heterologous Peptide Sequences in the C Terminus of Capsid Protein IX. J Virol. 2002 July; 76(14):6893-9] [Nadine C. Salisch, et al, Antigen capsid-display on human adenovirus 35 via pIX fusion is a potent vaccine platform. PLoS One. 2017; 12(3): e0174728].

In some embodiments of any aspect of the invention at least one modification in the capsid protein of the virus is within a major capsid protein or within a minor capsid protein. All of the adenovirus proteins on the outer surface of the virion (hexon, fiber, protein IX and penton) are capable of being modified.

In one embodiment, the modification in the major capsid protein is in the hexon protein.

In other embodiments, the modification in the minor capsid protein is in the pIX protein.

For any aspect of the present invention, at least one modification is the insertion or fusion of a first peptide partner to the capsid protein. Preferably, at least one modification is the insertion or fusion of a first peptide partner to the capsid protein via genetic modification of the adenovirus genome. Suitably, the modification is the insertion of a first peptide partner in the hexon protein, preferably into one or more of the HVR loops of the hexon protein. Thus, the insertion may be made into the sequence of the hexon protein, rather than fusing the first peptide partner to the N- or C-terminus. Suitably, the modification is the fusion of a first peptide partner in the pIX protein. For the pIX fusion, the first peptide partner may be fused at any suitable location, optionally the C- or N-terminal ends, preferably the C-terminal end. Those skilled in the art of modifying an adenovirus would be aware of how to introduce genetic modifications in order to modify the viral coat proteins as discussed herein.

In some embodiments of any aspect of the invention a covalent bond is an isopeptide. This allows irreversible conjugation of the first peptide partner to the second peptide partner forming covalently stabilised multi-protein complexes. This isopeptide bond may be spontaneous, i.e. without assistance, or require assistance, i.e. from a ligase. Specific peptide partner pairs are well known in the art, and includes the non-exhaustive list: SpyTag/SpyCatcher and derivatives and modifications thereof, SnoopTag/SnoopCatcher, DogTag/DogCatcher, SnoopTagJr/SnoopCatcher, SnoopTagJr/Dog Tag, Isopeptag/Pilin-C, Isopeptag-N/Pilin-N, SdyTag/SdyCatcher, PsCsTag/PsCsCatcher, RrgATagor RrgATag2/RrgACatcher and/or Jo/In and modifications and variants of any of these partner pairs.

In one embodiment, a first peptide partner may be DogTag, or SpyCatcher. Alternatively, a first peptide partner may be SpyTag. The adenoviral capsid protein inserted with DogTag is assigned the term “Ad-DogTag”. The adenoviral capsid protein inserted with SpyTag is assigned the term “Ad-SpyTag”. The adenoviral capsid protein fused with SpyCatcher is assigned the term “Ad-SpyCatcher”.

In other embodiments, a second peptide partner may be DogCatcher, SnoopTagJr, or SpyTag. In one example, a first peptide partner couples to a second peptide partner forming an isopeptide bond to make a peptide: peptide binding pair.

In relation to the data presented here, it is show that the insertion of DogTag into the hexon capsid protein performs well, and is capable of a good yield (FIG. 12).

In relation to the data presented here, the insertion of the following first peptide partners into pIX have been demonstrated as viable: SpyCatcher, SnoopCatcher, DogCatcher, SpyTag and SnoopTagJr. In FIG. 12 it is demonstrated that SpyCatcher fusions into pIX may be genetically less stable under certain circumstances, whilst SnoopCatcher and DogCatcher inserted into the same capsid protein may be more genetically stable, and therefore preferable under certain circumstances. However, SpyCatcher fusions are operable, see FIG. 11 where various entities are attached, whilst the virus retains infectivity. In FIG. 14 it is demonstrated that SnoopTagJr and SpyTag insertions into pIX do not adversely affect infectious yield, even when entities such as antigens are attached to these first peptide partners using the second peptide partner. It should be noted that the entire spike protein from Sars-CoV2 is over 500 KDa and is therefore a large attachment which has been successfully attached. Thus, SpyCatcher, SnoopCatcher, DogCatcher, SpyTag and SnoopTagJr may be inserted into pIX as the first peptide partner, most preferably SnoopCatcher, DogCatcher, SpyTag and SnoopTagJr may be inserted into pIX as the first peptide partner.

Examples of the peptide:peptide binding pair described herein includes but are not limited to SpyTag and SpyCatcher, DogTag and DogCatcher, SnoopTag/SnoopTagJr and SnoopCatcher; RrgATag/RrgATag2/DogTag and RrgACatcher, IsopepTag/IsopepTag-N and Pilin-C or Pilin-N, PsCsTag and PsCsCatcher; SnoopTagJr/SnoopTag and DogTag (mediated by SnoopLigase), and variants, derivatives and modifications of all these systems. Suitable peptide tag/binding partner pairs are described in detail in WO2011/09877, WO2016/193746, WO2018/18951 and WO2018/197854, herein incorporated by reference.

Accordingly, the second peptide partner may be linked or fused to an entity. This entity may be anything which it is desired to be attached to an adenoviral vector and is capable of being produced as a fusion to the second peptide partner. The type of entity will depend upon the prophylactic or therapeutic use to which the adenoviral vector will be put. The entity can be a targeting moiety, such as part of a ligand binding pair, an antibody or fragment thereof, or any other entity specifically recognised by a cell surface receptor. Such targeting moieties may be useful for oncolytic viruses and gene therapy applications. However, the entity may also simply have the function of blocking or shielding the adenovirus from the binding of antibodies present in the host in which the therapeutic is administered. Such a shielding entity may have no further function other than the provision of a shield to the adenoviral capsid core, enabling it to evade clearance. It is preferred that this shielding entity does not alter the tropism (natural infectivity) of the adenovirus. The inventors have observed that when the size of the second peptide partner with an attached shielding entity exceeds about 15 kDa, the combination appears to assist the modified adenovirus escape the immune response. This observation is recorded in FIG. 8A, where a partial shielding effect is noted for SnoopTagJr-AffiHER2. From gel migration, SnoopTagJr-AffiHER2 is considered to be about 15 kDa. This partial shielding effect may be advantageous. However, if the size of the combined second peptide partner plus attached shielding entity is greater than about 25 kDa, more complete shielding is observed, as seen in the results for DogCatcher-NANP9 (approximately 25-30 kDa) as seen in FIGS. 8B and 8C. In the case of a vaccine, the inventors have shown that the antigen may have a dual function, to raise an immune response in the host, but also to evade the host immune response to the adenovirus itself. Since it has not previously been possible to covalently attach entities of the size under consideration, this effect has not previously been observed.

Accordingly, the second peptide partner is linked or fused to an antigen or synthesised to include an antigen. The antigen may be attached to prevent future disease (prophylactic) or to assist the immune response in fighting a current disease (therapeutic). The antigen may be any suitable composition, including a peptide, polypeptide, protein, glycoprotein, lipoprotein, saccharide, polysaccharide, and the like. This antigen may be any suitable antigen, including self-antigen, cancer antigen, antigen that is an allergen, bacterial antigen, fungal antigen, viral antigen or an antigen from any pathogenic organism. The antigen may be a whole or entire antigen, or it may be a fragment thereof, such as an epitope. The antigen may be entirely natural or may be modified. The antigen may be a viral antigen or neoepitope or neoantigen. The antigen may be one common to a particular type of cancer, for example, such as shared neoepitope. The neoepitope or neoantigen may be patient-specific. Patient-specific antigens may arise from mutations in the antigen, for example change of coding sequence, frameshift mutation or altered post-translational modification. For example, wild type SnoopTagJr sequence has been synthesised as a fusion peptide to include a neoepitope sequence. In another example, DogCatcher is linked to an antigen to form a DogCatcher fusion.

In one embodiment, a first peptide partner is DogTag and a second peptide partner is DogCatcher. The attachment of a first peptide partner for example, DogTag attached to the hexon capsid protein forms the vector Ad-DogTag. Upon coupling to the second peptide partner DogCatcher linked to an antigen, the vaccine vector formed is called Ad-DogTag:DogCatcher-Antigen. In another embodiment, a first peptide partner is DogTag and a second peptide partner is SnoopTagJr or SnoopTag. The synthesis of SnoopTagJr with an antigen such as a neoepitope results in SnoopTagJr fusion. The synthesis of SnoopTag with an antigen such as a neoepitope results in SnoopTag fusion. The coupling of either SnoopTagJr or SnoopTag fusion to the vector Ad-DogTag results in the vector called Ad-DogTag:SnoopTagJr-fusion or Ad-DogTag:SnoopTag-fusion, respectively. This reaction requires SnoopLigase to facilitate the coupling between DogTag and SnoopTagJr fusion, or SnoopTag fusion. It may be preferred that DogTag is attached to the hexon capsid protein, optionally to a HVR loop. In some embodiments, the DogTag may be used to modify more than one HVR loop within the same hexon protein. This may increase the opportunity for attachment to the viral capsid.

In another embodiment, a first peptide partner is SpyCatcher and a second peptide partner is SpyTag. The SpyTag is linked to an antigen to form Ad-SpyCatcher:SpyTag-Antigen. Thus, it can be seen that the first peptide partner can be either what is termed in the art as a “tag” or “catcher”; with the second peptide partner component being the partner for this pair, the “catcher” or the “tag”, respectively. Herein both are designated as protein partners as they may be provided in either orientation, modifying the capsid protein or attached to the antigen. The coupling of the first peptide partner to the second peptide partner enables display of the antigens on the surface of the immunogenic adenoviral viral proteins. In this embodiment, the capsid protein is preferably pIX. Other “first peptide partner” and “second peptide partner” combinations that may be used in the context of pIX are primary partner SnoopTagJr and secondary partner SnoopCatcher, primary partner SpyTag and secondary partner SpyCatcher, primary partner SnoopCatcher and secondary partner SnoopTagJr or primary partner DogCatcher and secondary partner DogTag.

In another embodiment, the first peptide partner inserted into pIX may be any one of SnoopCatcher, DogCatcher, SpyTag and SnoopTagJr. These have all been demonstrated to be effective insertions, with good yield viability and are accessible for their respective second binding pair to allow decoration of the adenovirus with a desired entity. The respective second peptide partners are discussed here throughout. The resultant adenoviruses may be termed “Ad-SnoopCatcher”, “Ad-DogCatcher”, “Ad-SpyTag” and “Ad-SnoopTagJr”. Alternatively, these may be named “Ad pIX-SnoopCatcher”, “Ad pIX-DogCatcher”, “Ad pIX-SpyTag” and “Ad-pIX SnoopTagJr”

In some embodiments, the combination of the second peptide partner and the attached entity for attachment/decoration, such as an antigen is of significant size, for example it over 20 kDa, 25 kDa, 30 kDa, 40 kDa, 50 kDa, 60 kDa, 70 kDa, 80 kDa, 90 kDa, 110 kDa, 120 kDa, 130 kDa, 140 kDa, 150 kDa or 160 kDa, 170 kDa, 180 kDa, 190 kDa or more, such as over 200 kDa, over 300 kDa or over 400 kDa in size. Such decoration of a modified adenovirus is significant, since this type of addition is not possible using genetic fusion, due to the size limit imposed by the capsid proteins themselves. The inventors have shown that the addition of a second peptide partner entities of at least around 25-30 kDa has a beneficial effect of shielding the adenovirus from neutralising antibodies. Thus, it is preferred that the combined size of the entity attached to the second peptide partner is greater than 15 kDa, preferably greater than 20 kDa, optionally greater than 25 kDa in size, optionally at least 30 kDa in size, in order to provide a shielding effect.

The inventors have demonstrated that with larger entities (over about 30 kDa) attached to the adenoviral particle using the methods of the present invention, the decorated adenovirus of the present invention may evade the usual host immune response to the adenovirus. It is postulated that this is because the entity shields the adenovirus from antibody neutralisation by the host immune system. Evasion of host immunity to the adenoviral particle itself may be useful, since it would permit the adenovirus vector to be effective even in individuals who had previously been exposed to the parental adenovirus strain. Given that a large percentage of the human population is expected to have been previously infected by adenovirus, this would be a useful improvement over the prior art. Host immune clearance of prophylactic or therapeutic adenoviral vectors is a major obstacle to current therapeutics reaching the clinic. The present invention can therefore be used primarily as a way to shield any adenoviral vector from the host immune system. Advantageously, the entity attached using the technology can itself have a therapeutic effect, such as an antigen for a vaccine, or a targeting moiety for cell targeting in gene therapy or oncolytic viruses.

In other embodiments, DogTag is inserted into the surface loops of the hexon capsid protein. Hexon is the major component of the adenoviral capsid proteins with approximately 720 copies per virion. This enables a display of up to 720 ligands per virion, assuming that 100% of the hexon proteins are coupled with a peptide tag. DogTag is therefore the first peptide partner in some embodiments. In one embodiment, DogTag is inserted into at least one HVR loop of the hexon.

In other embodiments, SpyCatcher is fused to the C-terminus of adenovirus minor capsid protein pIX. The attachment of a first peptide partner for example, SpyCatcher fused to the pIX capsid protein forms the vector Ad-SpyCatcher. Upon coupling to the second peptide partner SpyTag linked to an antigen, the vaccine vector formed is called Ad-SpyCatcher:SpyTag-Antigen.

In another embodiment, the first peptide partner inserted into pIX may be any one of SnoopCatcher, DogCatcher, SpyTag and SnoopTagJr. The resultant adenoviruses may be termed “Ad-SnoopCatcher”, “Ad-DogCatcher”, “Ad-SpyTag” and “Ad-SnoopTagJr”. Alternatively, these may be named “Ad pIX-SnoopCatcher”, “Ad pIX-DogCatcher”, “Ad pIX-SpyTag” and “Ad-pIX SnoopTagJr”. Upon coupling to the respective peptide pairs, these may form: “Ad-SnoopCatcher: Snooptag”, “Ad-DogCatcher: DogTag”, “Ad-SpyTag:Spycatcher” and “Ad-SnoopTagJr:SnoopTag”. Any entity may be attached to the second peptide partner, such as an antigen for a vaccine composition.

As used herein the colon “:” refers to the presence of a covalent bond between the peptide pairs.

Accordingly, in another aspect of the invention there is provided a composition comprising an adenoviral vector in accordance with the invention. The composition is preferably immunogenic. Said adenoviral vector comprises a modified capsid protein, wherein said modification primes the vector for accepting the attachment of an entity. The modified capsid protein involves the fusion or insertion of a first peptide partner. A therapeutic or prophylactic virus may then be prepared by adding the requisite second peptide partner which is attached to an entity such as an antigen. Together the first and second peptide partners form a covalent bond either spontaneously or assisted by a third entity such as a ligase. Therefore, in the therapeutic or prophylactic composition, the capsid protein is modified by the inclusion of a first peptide partner covalently linked to a second peptide partner attached to an entity such as an antigen. If the entity attached is an antigen, the composition is a vaccine composition. This is a preferred use of the technology, since it capitalises on the highly immunogenic nature of the adenovirus itself Indeed, in FIG. 10 it can be seen that attaching the antigen to the vector (in this case a peptide antigen) is advantageous when compared to the results where the peptide and adenovirus are provided together as separate entities.

In another aspect of the invention there is provided a method of producing an adenoviral vector in accordance with the invention.

In another aspect there is provided a method of producing a vaccine comprising the mixing of an adenoviral vector in accordance with the invention with a second peptide partner attached to an antigen. Said method may require the use of a third or helper entity such as a ligase.

In a further aspect there is provided a method of producing an oncolytic virus comprising the mixing of an adenoviral vector in accordance with the invention with a second peptide partner attached to an entity such as a targeting moiety or shielding entity. Said method may require the use of a third or helper entity such as a ligase. The targeting moiety may be any suitable entity that permits the virus to be targeted to a cell or tissue type. Advantageously, said moiety also blocks the binding of host antibodies to the modified adenovirus. Alternatively, the shielding entity simply helps the already targeted oncolytic virus to escape the immune response.

In a further aspect there is provided a method of producing a gene therapy vector comprising the mixing of an adenoviral vector in accordance with the invention with a second peptide partner attached to a targeting moiety. Said method may require the use of a third or helper entity such as a ligase. The targeting moiety may be any suitable entity that permits the virus to be targeted to a cell or tissue type. Advantageously, said moiety also blocks the binding of host antibodies to the modified adenovirus. Those skilled in the art are aware of numerous gene therapy uses of adenovirus. The present invention may be used to shield vectors from the immune system and/or help with targeting of the vector.

In another aspect of the invention there is provided a vaccine comprising an immunogenic adenoviral vector in accordance with the invention for use in the prophylaxis and/or treatment of a disease. Suitably, said vaccine comprises an immunogenic adenoviral vector in accordance with any aspect or embodiment of the invention. Suitably, the vaccine is for use in mammals, including humans and animals. Suitably the vaccine is for use in humans, for example children, adults, women of reproductive age or pregnant women. In another aspect, the invention provides a method of inducing an immunogenic response, for example a protective immune response wherein the method comprises administering a composition in accordance with any aspect or embodiment of the invention. In a further aspect the invention provides a cancer vaccine which targets one or more tumour-specific mutations, permitting the specific targeting of an immune response to cancer cells. Personalised cancer vaccines are also contemplated. Such vaccines include one or more antigens that are specifically present on the cancer cells for that patient. This means that the vaccine is personalised to the particular cancer the patient has. These tumour-associated or cancer-associated antigens may be neoantigens, which are antigens newly expressed by the cancer cell.

In another aspect of the invention there is provided a kit comprising an adenoviral vector in accordance with the invention. The kit may also include the second peptide partner linked to an antigen, the second peptide partner ready for protein fusion to an antigen, or the genetic sequence for the second peptide partner to allow the genetic fusion of the second peptide partner to the gene for the antigen.

The present invention also extends to a kit for the preparation of a vaccine, said kit comprising an adenoviral vector as described herein. Also present in said kit may be the gene sequence for the second peptide partner, ready for fusing with the antigen. A further component may be instructions for use. Also included may be a third entity necessary for facilitating the covalent bond between the peptide partner pair.

In another aspect of the invention there is provided a kit comprising an adenoviral vector in accordance with the invention. The kit may also include the second peptide partner linked to a targeting moiety, the second peptide partner ready for protein fusion to a targeting moiety, or the genetic sequence for the second peptide partner to allow the genetic fusion of the second peptide partner to the gene for the targeting moiety.

The present invention also extends to a kit for the preparation of an oncolytic virus, said kit comprising an adenoviral vector as described herein. Also present in said kit may be the gene sequence for the second peptide partner, ready for fusing with the targeting moiety. A further component may be instructions for use. Also included may be a third entity necessary for facilitating the covalent bond between the peptide partner pair. The adenoviral vector in this instance would be replication competent and may be modified to include genes to ensure destruction of the infected cancer cell.

The present inventors are aware that peptide binding partners have previously been used in vaccine preparation, in particular for use in virus-like particles. However, this technology is distinct from the use of a viral vector, which may have a different utility in vaccine preparation due to the immunological response the viral vector itself generates. A viral vector is efficient at gaining entry to the cytoplasm and often the nucleus as well, which leads to an enhanced cytotoxic T cell response to viral vectors, compared to virus like particles. For many infectious diseases and for cancer, the cytotoxic T cell response is a major component of protective immunity. Virus like particles (VLPs) resemble viruses in their size (approx. 20-200 nm), their shape and their repetitive protein arrangement but lack any genetic material from a pathogen. Therefore, the method of their synthesis and the challenges faced in preparing vaccines from VLPs is very different to the method of preparation and the challenges faced with preparing a vaccine from a viral vector. These peptide partner pairs have not previously been used or suggested for use for modifying a capsid protein in an immunogenic viral vector.

Further, the inventors are aware that others have used peptide binding partners, most notably SpyTag, to modify the surfaces of other viruses. The other viruses selected for modification by other groups present different challenges to those at hand with adenovirus. Primarily, the majority of viruses previously modified are enveloped. Thus, the modifications are taking place on proteins that are part of the lipid envelope. In adenovirus, the present inventors have found that modifying capsid proteins in a non-enveloped virus is not as straightforward to do as expected, and that structural constraints mean that it is not possible in some instances to include the modification and retain infectivity to a satisfactory level. Further, the previous work conducted to modify viruses with peptide partner pairs that form a covalent bond has focused on re-targeting the virus or altering tropism. This is different to the main focus of the present application, which is the correct display of antigens on the capsid surface in order to raise an immune response.

### Detailed Description of the Invention

### FIGURES

FIG. 1 (A-D). Modular covalent decoration of the adenovirus capsid via insertion of SpyTag or DogTag into hexon HVR loops.

FIG. 1 shows four representations of embodiments of the present invention. FIG. 1A) Reaction of SpyCatcher with SpyTag via an isopeptide bond. FIG. 1B) Reaction of DogCatcher with DogTag via an isopeptide bond. FIG. 1C) Coupling reaction between SnoopTagJr and DogTag, catalysed by SnoopLigase. FIG. 1D) Modular hexon decoration, showing surface display of DogTag on the viral capsid and subsequent coupling of peptides or protein antigens via SnoopTagJr or DogCatcher. FIG. 1E) Multiple sequence alignment of the adenovirus hexon protein indicating location of hypervariable region (HVR) loops 1, 2 and 5 and the deletions generated at the insertion site prior to insertion of SpyTag or DogTag at each locus. Shaded residues indicate differing residue identity to Ad5. Hexon sequences from the following Human adenovirus serotypes are shown; Ad5 and Ad2 (species C), Ad12 and Ad18 (species A), Ad3 and Ad35 (species B), Ad4 (species E), Ad40 and Ad41 (species F). Note that the insertions sites indicated are identical between SpyTag and DogTag recombinants at each of the three HVRs tested. At each locus, SpyTag or DogTag sequences are flanked by GSGGSG sequences.

FIG. 2 (A-D). Reactivity of SpyTag following insertion into Ad5 HVR loops. FIG. 2A: SDS-PAGE and Coomassie staining analysis of Ad5 displaying SpyTag in the loop HVR1, HVR2 or HVR5 (1E+10 viral particles) incubated with SpyCatcher at 4° C. for 16 h. FIG. 2B: Vector infectivity assay of the samples shown in FIG. 2A. Data show infectious units (ifu) per ml mean+range for duplicate wells. FIG. 2(C-D): Ad5(GFP) vectors displaying SpyTag at HVR1 (Ad5(GFP)HVR1-SpyTag) (1E+10 viral particles) were incubated with biotinylated SpyCatcher at 15 μM or 40 μM under different conditions as shown. FIG. 2C: SDS-PAGE Coomassie staining analysis of Ad5 displaying SpyTag incubated with SpyCatcher at 20° C. or 37° C. for 3 hours or at 20° C. or 4° C. for 16 hours. FIG. 2D: Vector infectivity assay was performed on the same samples shown in FIG. 2C. Data show mean+range for duplicate wells.

FIG. 3 (A-D). Reactivity of DogTag following insertion into Ad5 HVR loops. FIG. 3A: Yield comparison of Ad5 vectors displaying DogTag on hexon surfaces (in HVR 1, 2 or 5) versus Ad5 vectors with an unmodified hexon (WT). P:I ratios (ratio of total viral particles calculated by UV spectrophotometry to infectious units calculated by GFP focus assay) for each vector batch are indicated above each bar. FIG. 3B: SDS-PAGE and Coomassie staining analysis of Ad5 displaying DogTag at HVR1, HVR2 or HVR5 (1E+10 viral particles) incubated with DogCatcher (5 μM) at 4° C. for 16 h. FIG. 3C: Vector infectivity assay was performed on the samples shown in FIG. 3B. Data show mean+SD of triplicate wells. FIG. 3D: SDS-PAGE and Coomassie staining of Ad5 displaying DogTag incubated with DogCatcher for 0.1, 1 or 16 hours at 4° C.

FIG. 4 (A-C). Reactivity of Ad5 HVR-DogTag with SnoopTagJr directed by SnoopLigase. FIG. 4A: Reactivity of SnoopTagJr fused to an affibody to HER2 (SnJr-AffiHER2) with DogTag inserted into different HVR loops, catalysed by SnoopLigase, assessed by SDS-PAGE and Coomassie staining. FIG. 4B-C: Temperature-dependence of reactivity of SnJr-AffiHER2 with Ad5(GFP)HVR5-DogTag. FIG. 4B: SDS-PAGE and Coomassie staining analysis of the reaction between SnJr-AffiHER2 and Ad5(GFP)HVR5-DogTag, catalysed by SnoopLigase at 4° C. or 20° C. FIG. 4C: A vector infectivity assay was performed on the same samples described in FIG. 4B. Data show mean+range of duplicate wells. FIG. 4D: SDS-PAGE and Coomassie staining analysis on the effect of glycerol (15% w/v) and salt addition on SnoopLigase-mediated reactivity.

FIG. 5 (A-B). Assessment of vector infectivity using an anti-hexon immunostaining assay for non-fluorescent recombinant vectors. FIG. 5A: Anti-hexon immunostaining of HEK293A cells infected with Ad5 vectors with a WT hexon protein or Ad5 vectors displaying DogTag at HVR5. Brightfield microscopy images using a 4× objective, with hexon-positive cells shown in black. Scale bar=1000 μm. FIG. 5B: Comparison of the infectious titre for Ad5(GFP) hexon WT and Ad5(GFP) HVR5 DogTag using both GFP fluorescence and hexon immunostaining assays. Both assays were performed using the same wells on the same plate, n=4. A two-tailed t-test was performed, but no statistically significant difference was observed between groups. Bars show mean±SD; dots represent spot counts from individual wells.

FIG. 6 (A-F). Reactivity of DogTag inserted into hexon loops with SnoopTagJr tagged peptides. FIG. 6A: Illustration of a competition assay using DogCatcher (DC) to assess the efficiency of SnoopTagJr-peptide (SnJr-peptide) coupling to hexon-DogTag (hexon-DT), catalysed by SnoopLigase (SnL). FIG. 6B: SDS-PAGE and Coomassie analysis of the coupling efficiency of SnoopTagJr-hTERT peptide to hexon-DT using the DogCatcher competition assay. FIG. 6C: SDS-PAGE and Coomassie analysis of the coupling efficiency of SnoopTagJr-SIINFEKL peptide (PEP1; SnJr-GGS-SIINFEKL. PEP2; SnJr-AAY-SIINFEKL) to hexon-DT using the DogCatcher competition assay. FIG. 6D: Illustration of assessment of SnJr-peptide coupling to hexon-DT using monovalent streptavidin (mSA) direct gel shift assay with Western blotting using anti-hexon antibody. If added after boiling, the biotin-streptavidin interaction is stable on SDS-PAGE. FIG. 6E: SDS-PAGE and Coomassie analysis of SnJr-biotin coupling to Ad5-DT using the direct gel shift assay with mSA. FIG. 6F: A vector infectivity assay was performed on the same samples shown in FIG. 6C. Bars show mean and range of duplicate wells.

FIG. 7 (A-C). Coupling of Ad5 HVR DogTag to DogCatcher fusion proteins. FIG. 7A: Illustration of three DogCatcher fusion constructs; NANP9, NANP18, and NANPD, derived from the Plasmodium falciparum circumsporozoite protein (PfCSP), isolate 3D7. NDVP and NANP tetrapeptide repeats are illustrated. FIG. 7B: SDS-PAGE and Coomassie analysis of the reactivity of DogCatcher-NANPn with DogTag inserted into hexon HVR5 loop. FIG. 7C: A vector infectivity assay was performed on the same samples shown in FIG. 7B. Bars show mean and range of duplicate wells. Sizes of surface decoration: DogCatcher-NANP9 (^{˜}25-30 kDa), DogCatcher-NANP18 (^{˜}35-40 kDa) and DogCatcher-NANP Domain (^{˜}60 kDa)

FIG. 8 (A-C). Coupling via isopeptide bonds to the surface of Ad5 via hexon reduces the potency of virus-neutralising antibodies. FIG. 8A: Coupling via SnoopLigase to the surface of Ad5 via hexon reduces the potency of virus-neutralising antibodies. Adenovirus was added to cells in the presence of varying concentration of the monoclonal antibody targeting the adenovirus hexon (mAb 9C12). Productive adenovirus infection was detected from the fluorescence of adenovirus-encoded GFP expressed in the cells. Intensity of fluorescence of Ad5-DT alone, Ad5-DT+SnJr-AffiHER2 and Ad5-DT+SnL+SnJr-AffiHER2. FIG. 8B: Coupling of DogCatcher-NANPn to the surface of Ad5 via hexon reduces the potency of virus-neutralising antibodies, analysed as in FIG. 8A. Intensity of fluorescence from Ad5-DT alone, or DogCatcher-NANPn coupled to Ad5 DogTag. FIG. 8C: Adenovirus was added to cells in the presence of varying dilutions of adenovirus-neutralising serum. Productive adenovirus infection was detected from the fluorescence of adenovirus-encoded GFP expressed in the cells. Intensity of fluorescence of Ad5-DT alone, or DogCatcher-NANPn coupled to Ad5 DogTag. In A-C, bars indicate mean and range of duplicate values.

FIG. 9: Human coagulation Factor X-dependent vector transduction of SKOV3 cells. Ad5(GFP)-DogTag (Ad5) or Ad5(GFP)-DogTag:DogCatcher-NANP18 (Ad5-NANP18) vectors (2E+9 viral particles) were incubated in the presence or absence of human coagulation Factor X (8 μg/mL) on SKOV3 cells for 2 h at 37° C. in serum-free media. Then media was replaced with fresh complete media and plates were incubated for a further 48 h. Infectious titres were then calculated by enumeration of GFP-positive foci by fluorescence microscopy. Samples were plated in duplicate and bars show mean and range of data values.

FIG. 10 (A-C). Ad5(GFP) vectors displaying SIINFEKL peptides on the capsid surface generate CD8+T cell responses against both the capsid-displayed peptide and internally-encoded GFP antigens. FIG. 10A: Design and immunisation schedule for mouse immunogenicity experiment to assess CD8⁺ T cell responses to Ad5 surface-displayed peptide antigens. To confirm the importance of physical attachment of peptide epitopes to the capsid surface, SIINFEKL specific T cell responses after administration of Ad5(GFP) HVR5 DogTag with SIINFEKL attached (Ad5-DT:SIINFEKL) were compared to co-administration of Ad5(GFP) HVR5 DogTag (Ad5-DT) with free SIINFEKL peptide (Groups 1 and 2, vs Group 3), and to SIINFEKL peptide with poly I:C adjuvant (Group 4). FIG. 10B: Spleen ex vivo IFNγ-ELISPOT responses to SIINFEKL (spot forming cells per million splenocytes). FIG. 10C: Spleen ex vivo IFNγ-ELISPOT responses to the epitope DTLVNRIEL (EGFP₁₁₈₋₁₂₆). In FIG. 10B and FIG. 10C, spots represent responses in individual animals, while bars represent median values with range.

FIG. 11 (A-D). Modular covalent decoration of the adenovirus capsid via fusion of SpyCatcher at the C-terminus of pIX. FIG. 11A: Modular adenovirus capsid decoration, showing surface display of SpyCatcher on the viral capsid via fusion to pIX, and subsequent coupling of peptide or protein antigens via SpyTag. FIG. 11B: SDS-PAGE analysis of Ad5(GFP) pIX-SpyCatcher coupled with SpyTag-Maltose binding protein (SpyTag-MBP, 5 μM) or SpyTag-Human cytomegalovirus (HCMV) pentamer (SpyTag-Pentamer, 2.5 μM) for 16 h at 4° C. gH is the pentamer subunit containing the SpyTag. Proteins detected by Western blotting using anti-SpyCatcher polyclonal mouse sera. FIG. 11C: SDS-PAGE analysis of Ad5(GFP) pIX-SpyCatcher coupled to SpyTag-peptides. Monovalent streptavidin (mSA) was added to induce a clear gel shift, and proteins detected by Western blotting using anti-SpyCatcher polyclonal mouse sera. FIG. 11D: Vector infectivity assay was performed on the same samples as shown in FIG. 11B and FIG. 11C. Bars show mean and SD of triplicate wells.

FIG. 12 (A-B). Viability, genetic stability, and yield of different recombinant adenovirus vectors with SpyCatcher, SnoopCatcher or DogCatcher fused to the C-terminus of pIX. FIG. 12A: Schematic representation of the pIX amino acid sequence for different Ad5(GFP) vectors with versions of SpyCatcher, SnoopCatcher or DogCatcher fused to the C-terminus of pIX, with corresponding information regarding vector viability in HEK293A cells and genetic stability (after >3 passages). SpyCatcher dN1 (delta N1) refers to the same N-terminally truncated version of SpyCatcher used in the other Figures from this document. SpyCatcher dN1dC2 (delta N1, delta C2) refers to a shorter version of SpyCatcher with an additional C terminal truncation. FIG. 12B: Yield comparison of viable Ad5 vectors (as indicated in FIG. 12A) displaying SpyCatcher, SnoopCatcher or DogCatcher at the C-terminus of pIX. Yields of vectors with pIX-Catcher fusions are compared to Ad5(GFP) HVR5 DogTag, using equivalent volumes of cultured cells and the same vector harvest/purification protocols. For SnoopCatcher and DogCatcher fusions, bars indicate mean and range of n=2 batches.

FIG. 13 (A-C). Reactivity of SnoopCatcher and DogCatcher fused to the C-terminus of pIX. FIG. 13A: Reactivity of Ad5(GFP) pIX-SnoopCatcher vector with SnoopTagJr-fused to the full length spike protein from SARS CoV2 (Spike) or SnoopTagJr fused to the receptor binding domain (RBD) of SARS CoV2 spike. Ad5(GFP) pIX-SnoopCatcher (3E+9 viral particles) was incubated alone or co-incubated with Spike-SnoopTagJr (0.75 μM) or RBD-SnoopTagJr (5 μM) for 16 h at 4° C. Samples were run on SDS-PAGE and western blotting performed using polyclonal mouse sera with reactivity against SnoopCatcher. Species representing pIX-SnoopCatcher (pIX-SnC), pIX-SnoopCatcher:SnoopTagJr-RBD (pIX-SnC:SnJr-RBD) and pIX-SnoopCatcher:SnoopTagJr-Spike (pIX-SnC:SnJr-Spike) are indicated. FIG. 13B: Vector infectivity assay was performed using the same samples shown in FIG. 13A. Bars show mean and SD of triplicate samples. FIG. 13C: Reactivity of Ad5(GFP) pIX-DogCatcher vector with DogTag-fused to Small Ubiquitin Modifier (SUMO). Ad5(GFP) pIX-DogCatcher (1E+9 viral particles) was incubated alone or co-incubated with SUMO-DogTag (17 μM) for 16 h at 4° C. Samples were run on SDS-PAGE and western blotting performed using polyclonal mouse sera with reactivity against DogCatcher. Species representing pIX-DogCatcher (pIX-DC) and pIX-DogCatcher:DogTag-SUMO (pIX-DC:DT-SUMO) are indicated.

FIG. 14 (A-C). Reactivity of SnoopTagJr and SpyTag fused to the C-terminus of pIX. FIG. 14A: Yield comparison of Ad5 vectors displaying SnoopTagJr or SpyTag fused to the C-terminus of pIX. Both vectors have GGS (EAAAK)3 GS linker sequences between pIX and Tag. Yields of vectors in HEK293A cells with pIX-Catcher fusions are compared to Ad5(GFP) HVR5 DogTag, using equivalent volumes of cultured cells and the same vector harvest/purification protocols. FIG. 14B: Reactivity of Ad5(GFP) pIX-SnoopTagJr with SnoopCatcher fused to SARS CoV2 RBD and reactivity of Ad5(GFP) pIX-SpyTag with SpyCatcher fused to SARS CoV2 RBD. Vectors (1E+10 viral particles) were incubated alone, or co-incubated with RBD-SnoopCatcher, SnoopCatcher-RBD, or RBD-SpyCatcher (all 3.5 μM) for 16 h at 4° C. Samples were run on SDS-PAGE and western blotting performed using a polyclonal anti-RBD antibody. Species representing pIX-SnoopTagJr:SnoopCatcher-RBD (pIX-SnJr:SnC-RBD), pIX-SpyTag:SpyCatcher-RBD (pIX-ST:SC-RBD) and non-coupled SnoopCatcher-RBD/SpyCatcher-RBD (SnC/SC-RBD) are indicated and labelled (*). FIG. 14C: Vector infectivity assay was performed using the same samples shown in FIG. 14B. Bars show mean and range of duplicate samples.

### ADENOVIRUS

Adenoviruses (Ad) are a non-enveloped double stranded DNA virus with a genome of approximately 36 kilobases (kb). There are over 60 human adenovirus serotypes grouped into species A-G. Each group comprises of a number of adenoviral serotypes, for example, the subgroup species C includes Ad5 and Ad2. Ad5 is the most extensively studied serotype, and the most widely used platform for the development of oncolytic viruses. In the development of oncolytic viruses, it is desirable to be able to target particular tissues, and therefore the tropism may be altered. A major issue with using some adenovirus serotypes, including Ad5, in clinical settings is the pre-existing immunity in humans. Adenoviruses are typically 70-90 nm in size with an icosahedral capsid shape. The outer capsid structure, also known as ‘capsid protein’ comprises three major types of protein (hexon, fiber and penton base). There are additional minor proteins in the outer capsid including VI, VIII, IX, IIIa and IVa2. Hexon is the major component of the adenoviral capsid accounting for more than 83% of the capsid protein. Hexon modification has been shown to allow for circumvention of pre-existing neutralising antibodies in some circumstances, including the swapping of HVR from different serotypes.

### Adenovirus for Modification

Adenovirus can be replication-defective: certain genes are deleted from the genome in order to ensure that when the adenovirus is used as a therapeutic, it is no longer capable of replication. This may result from the deletion of a set of genes from the genome, and is within the skills of those working with adenoviruses. This may be an advantage for use in vaccines, where the aim of the adenoviral vector is to present the antigen to the immune system in a format that makes it highly immunogenic, while limiting cytotoxicity. However, for other applications such as oncolytic viruses, being replication-competent is key. Replication-competent adenoviruses may still contain some modifications to prevent replication in normal cells, for example by deletion of a key gene. Oncolytic Ad vectors lyse cancer cells at the end of their life cycle and it is important that the progeny do not infect normal tissue. Cancer cells are generally more permissive for adenoviral replication, whilst normal cells require the adenovirus to have a full complement of genes to assist replication.

The adenovirus may be from any serotype or strain of adenovirus. Therefore, suitable adenoviruses for modification may come from those that infect mammals other than humans, in order to minimise prior exposure effects. The capsid structure is strongly conserved, and therefore the adenoviral serotypes and species may be interchangeable.

The adenovirus may be any modified adenovirus. Thus, the modified adenovirus may additionally encode antigens for example. These encoded antigens would then be expressed after infection. This provides the possibility of a multi-faceted prophylactic or therapeutic, such that an antigen can be displayed on the surface of the virus and another antigen expressed upon vector transduction using host cell machinery. Thus, the adenovirus may be genetically modified, such that, for example it includes a transgene. This transgene is designed for delivery to the host cell and may be a gene encoding an antigen, for example.

### Adenovirus-Mediated Infectivity

Adenovirus infectivity in cells that express the Coxsackievirus and adenovirus receptor (CAR) is mediated via the fiber protein. An example of a cell line that expresses the CAR receptor is HEK293 cells. Fiber binds to the CAR receptor on the surface of cells and this mediates the initial attachment of the virus. However, it was recently demonstrated that instead of a fiber-mediated entry of the adenovirus, Factor X (FX)—a coagulation factor present in human serum can bind to the hexon proteins of some adenovirus serotypes to facilitate the entry of the virus in some cell types. An example of a cell line that mediates infection via the hexon protein is SKOV3. It is believed that FX mediated infection via the adenovirus hexon can enhance liver tropism of adenovirus vectors in vivo. Modifications of the hexon protein such as insertion of DogTag and coupling to an antigen reduces hexon-mediated infectivity of the cells, as demonstrated in the Examples. This is a desirable effect as the natural tropism of adenovirus when injected intravenously can cause liver toxicity in patients at very high doses. Reduction of hexon-mediated infectivity to reduce liver toxicity would be advantageous to the present invention.

### Hexon Capsid Protein

The hexon capsid protein is approximately 100 kDa in size, with 720 monomers per virion. Hexon monomers organise into trimers so that 12 lie on each of the 20 facets, resulting in 240 trimers per virion. Hexon sequences contain hypervariable regions (HVR) corresponding to loops on the external surface on the virus and therefore cover almost the entire surface of the virus. Each monomer has seven HVRs identified as HVR1-HVR7 which are serotype specific. As the loops are on the external surface of the virus, hexon loops are the main antigen recognition site, a target for host immune responses. Hexon protein varies in length, for example, Ad2 is the longest known hexon protein with a length of 968 amino acids (UniProt ID: P03277). Ad5, the most commonly used adenovirus for gene therapy has a length of 952 amino acids (UniProt ID: P04133). Modifying hexon HVRs which contain the serotype-specific epitope seems to be a promising approach to overcome the host neutralisation response. Any one of the HVRs could be modified. Exemplified herein, modifications were successfully made to HVR1, HVR2 and HVR5, surprisingly using DogTag. When the hexon protein was modified according to the invention and an antigen was attached via the peptide partner pair, neutralisation by anti-adenovirus neutralising antibodies was reduced.

### pIX Capsid Protein

pIX protein is a minor capsid protein which is approximately 14.3 kDa in size. There are approximately 240 pIX monomers per virion. The pIX protein functions to stabilise the hexons on the viral surface. The C-terminus of the pIX protein is exposed on the surface of the virus and is therefore a desirable site for fusion of small and large peptides. Ad5 pIX has two domains connected by a flexible linker. The Ad5 pIX protein has a length of 196 amino acids (UniProt ID: Q2KS03).

### Modifications to the Capsid Proteins

Modification to the capsid proteins can be genetic or non-genetic, including chemical. The capsid proteins can be genetically modified through the incorporation of antigens into the capsid. Alternatively, the viral particle surface may be directly modified. Modification of all three major capsid proteins has been demonstrated previously. However, the results from these modifications has been mixed, and there is a major obstacle in the size of the insert that the most promising approaches offer, particularly regarding modification of hexon.

“At least one modification” as used herein refers to the inclusion of a first peptide partner insertion into the viral capsid protein using any appropriate means. For example, the insertion of the first peptide partner into the adenoviral hexon loops or the fusion of a first peptide partner to the adenoviral pIX minor capsid protein. This modification may be made genetically through gene fusion, for example, or chemically.

### Insertion

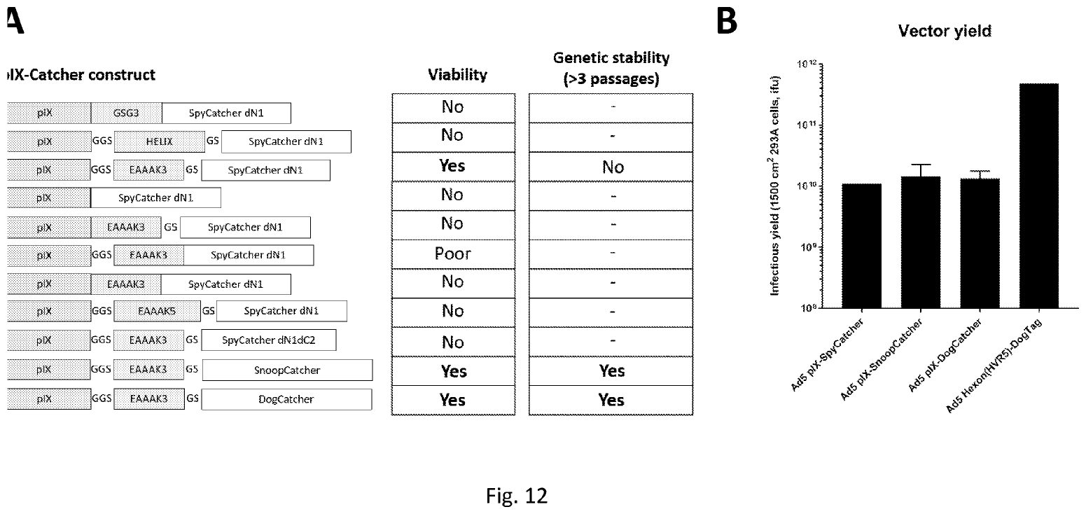

The first peptide partner may be directly inserted into the relevant capsid protein. As discussed herein, such an insertion is achieved by genetic manipulation. Alternatively, the first peptide partner can be inserted with a peptide sequence of any suitable length which separates the first peptide partner from the capsid protein. This peptide sequence may be described as a linker sequence, a spacer sequence, a structural sequence such as a helix, or even a hinge sequence. Linker or spacer sequences may simply separate the capsid protein and the first peptide partner and act as a “link” between the two entities. A structural sequence may provide a physical separation of the first peptide partner from the capsid protein. A hinge sequence may act as a linker between the capsid protein and the first peptide partner but permit a degree of motion to occur, such that the first peptide partner can move relative to the capsid protein. Various linkers, spacers and hinge sequences are exemplified herein, most notably those depicted in FIG. 12.

Where the insertion is into a loop of sequence, the first peptide partner may be separated from the capsid protein on one or both sides, i.e. it may be flanked by sequences which are linkers, spacers, structures or hinges. Each flanking side may be the same or different.

### Peptide Partner Pairs

Proteins that are capable of spontaneous isopeptide bond formation (so-called “isopeptide proteins”) have been advantageously used to develop peptide partner pairs (i.e. two-part linkers) which covalently bind to each other and provide irreversible interactions (see e.g. WO2011/098772 and WO 2016/193746 both herein incorporated by reference, together with WO2018/189517 and WO2018/197854 both incorporated herein by reference). In this respect, proteins which are capable of spontaneous isopeptide bond formation may be expressed as separate fragments, to give a first peptide partner and a second peptide partner which is the peptide binding partner for the first peptide partner, where the two fragments are capable of covalently reconstituting by isopeptide bond formation. This covalent reconstitution links molecules or components fused to the second peptide partner and the requisite first peptide partner. The isopeptide bond formed by the peptide partner pair is stable under conditions where non-covalent interactions would rapidly dissociate, e.g. over long periods of time (e.g. weeks), at high temperature (to at least 95° C.), at high force, or with harsh chemical treatment (e.g. pH 2-11, organic solvent, detergents or denaturants).

Isopeptide bonds are amide bonds formed between carboxyl/carboxamide and amino groups, where at least one of the carboxyl or amino groups is outside of the protein main-chain (the backbone of the protein). Such bonds are chemically irreversible under typical biological conditions and they are resistant to most proteases. As isopeptide bonds are covalent in nature, they result in some of the strongest measured protein-protein interactions.

In brief, a two-part linker, i.e. a peptide partner pair (a so-called peptide tag/binding partner or catcher pair) may be derived from a protein capable of spontaneously forming an isopeptide bond (an isopeptide protein), wherein the domains of the protein are expressed separately to produce a peptide “tag” that comprises one of the residues involved in the isopeptide bond (e.g. an aspartate or asparagine, or a lysine) and a peptide or peptide binding partner (or “catcher”) that comprises the other residue involved in the isopeptide bond (e.g. a lysine, or an aspartate or asparagine) and at least one other residue required to form the isopeptide bond (e.g. a glutamate). Mixing the peptide tag and binding/catcher partner results in the spontaneous formation of an isopeptide bond between the tag and binding partner. Thus, by separately incorporating the peptide tag and binding partner into different molecules or components, e.g. proteins, it is possible to covalently link said molecules or components together via an isopeptide bond formed between the peptide tag and binding partner, i.e. to form a linker between the molecules or components incorporating the peptide tag and binding partner.

The spontaneous formation of the isopeptide bond may be in isolation, and not require the addition of any other entity. For some peptide tag and binding/catcher partner pairs, the presence of a third or helper entity, such as a ligase, may be required in order to generate the isopeptide bond.

A peptide tag/binding partner pair (two-part linker), termed SpyTag/SpyCatcher, has been derived from the CnaB2 domain of the Streptococcus pyogenes FbaB protein (Zakeri et al., 2012, Proc Natl Acad Sci USA 109, E690-697) and used in diverse applications including vaccine development (Brune et al., 2016, Scientific reports 6, 19234; Thrane et al., 2016, Journal of Nanobiotechnology 14, 30).

Variants, derivatives and modifications of the binding pairs may be made by any suitable means. Variants, derivatives and functionally operative modifications may involve amino acid additions, substitutions, alterations or deletions that retain the same function in relation to the ability to form an isopeptide bond with the relevant binding partner.

For some of the binding pairs, mediation by a third entity such as an enzyme is required. For example, SnoopLigase may be used to mediate the bond formation between SnoopTagJr/SnoopTag and DogTag. Thus, the pairing may require the assistance of an enzyme such as a ligase.

It will be understood that as used herein, either the first peptide partner or the second peptide partner may be the peptide “tag” and the other is the “binding partner/catcher”.

Suitably, the first and second peptide partners form the peptide partner pair termed SpyTag/SpyCatcher. Suitably, the SpyCatcher component is DeltaN1 (ΔN1) SpyCatcher (as described in Li, L., Fierer, J. O., Rapoport, T. A. & Howarth, M. Structural analysis and optimization of the covalent association between SpyCatcher and a peptide Tag. J. Mol. Biol. 426, 309-317 (2014)) which has a 23 amino acid truncation at the N-terminus compared to “SpyCatcher”.

In other embodiments, the first and second peptide partners form a peptide partner pair which is a mutated version of SpyTag/SpyCatcher displaying an increased rate of reaction for isopeptide bond formation such as, for example, those described in co-pending application, GB1706430.4. In some embodiments, these mutated forms may be useful for the attachment of large proteins (e.g. >50 kDa or >100 kDa, such as the >160 kDa HCMV pentameric protein exemplified herein) and/or where slow reactions or steric hindrance may be an issue.

In other embodiments, the isopeptide proteins forming the peptide partner pair may include SnoopTag/SnoopCatcher, described, for example in WO 2016/193746.

In some embodiments, one or both of the isopeptide proteins forming the peptide partner pair may have N- or C-terminal truncations, whilst still retaining the reactivity of the isopeptide bond.

Exemplary first and second peptide partner pairs (peptide tag/binding partner pairs; reactive pairs) are described in the following table:

Reactive pairs

| | | |
|---|---|---|
| (a) | SpyTag | SpyCatcher |
| | SpyTag002 | |
| | SpyTag002 RG T3H | |
| (b) | SpyTag | SpyCatcher002 |
| | SpyTag002 | |
| | SpyTag002 RG T3H | |
| (c) | SpyTag | SpyCatcher002 D5A A92P Q100D |
| | SpyTag002 | |
| | SpyTag002 RG T3H | |
| (d) | SnoopTag | SnoopCatcher |
| | SnoopTagJr | |
| (e) | RrgATag | RrgACatcher - denoted here also as |
| | RrgATag2 | DogCatcher |
| | DogTag | |
| (f) | Isopeptag | Pilin-C |
| (g) | Isopeptag-N | Pilin-N |
| (h) | PsCsTag | PsCsCatcher |
| (i) | SnoopTagJr | DogTag [mediated by SnoopLigase] |
| | SnoopTag | |

These entities are described, for example, in WO2011/098772, WO2016/193746, GB1706430.4 GB 1705750.6 or Li L., et al., J. Mol. Biol. 426, 309-317 (2014).

Variants, derivatives and modifications of the binding pairs may be made by any suitable means. Variants, derivatives and functionally operative modifications may involve amino acid additions, substitutions, alterations or deletions that retain the same function in relation to the ability to form an isopeptide bond with the relevant binding partner.

For some of the binding pairs, mediation by a third entity such as an enzyme is required. For example, SnoopLigase may be used to meditate the bond formation between SnoopTagJr and DogTag. Thus, the pairing may require the assistance of an enzyme such as a ligase.

### Antigen

An antigen as used herein refers to any molecule that is capable of inducing immune responses. An antigen can be a self-antigen, cancer antigen, allergenic antigen, tumour antigen, viral antigen, bacterial antigen, parasitic antigen or fungal antigen. A tumour antigen includes tumour-specific antigen, tumour-associated antigen and neoantigens, newly formed antigens by cancerous cells. “Tumour-specific antigen” refers to antigens that are only found on tumour cells. “Tumour-associated antigen” refers to antigens presented by both tumour and normal cells. “Neoantigen” refers to newly formed antigens by tumour cells. “Antigen” as used herein includes peptides and epitopes, variants and derivatives thereof.

Tumour-associated antigens include, but are not limited to adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (CEA), CAGE 1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin DI, Cyclin-AI, dek-can fusion protein, DKK1, EFTUD2, Elongation factor 2, ENAH (hMena), Ep-CAM, EpCAM, EphA3, epithelial tumor antigen (ETA), ETV6-AML1 fusion protein, EZH2, ErbB receptors, E6, E7, FGF5, FLT3-ITD, FN1, G250/MN/CAIX, GAGE-1,2,8, GAGE-3,4,5,6,7, GAS7, glypican-3, GnTV, gp100/Pmel 17, GPNMB, HAUS3, Hepsin, HER-2/neu, HERV-K-MEL, HLA-A11, HLA-A2, HLA-DOB, hsp70-2, HPV E2, HPV E6, HPV E7 antigen, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferase fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class 1, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (PEM), PPPIR3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPDI, SOX10, Spl7, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-I/gp75, TRP-2, TRP2-INT2, tyrosinase (TYR), VEGF, WT1, XAGE-Ib/GAGED2a,

Those skilled in the art of identifying tumour-associated antigens will appreciate that new antigens, including neoantigens, are continually identified, and as such this list is not exhaustive.

Viral antigens include, but are not limited to antigens of the following viruses or class of viruses; Human Papilloma Viruses (HPV), Human Immunodeficiency virus (HIV), Herpes Simplex Virus (HSV2/HSV1), Influenza virus (types A, B and C), Polio virus, Respiratory Syncitial Virus (RSV), Rhinoviruses, Rotaviruses, Hepatitis A virus, Norwalk Virus Group, Enteroviruses, Astroviruses, Measles virus, Parainfluenza virus, Mumps virus, Varicella-Zoster virus, Human Cytomegalovirus (HCMV), Epstein-Barr virus, Adenoviruses, Rubella virus, Human T-cell Lymphoma type I virus (HTLV-I), Hepatitis B virus (HBV), Hepatitis C virus (HCV), Hepatitis D virus, Poxviruses, Marburg virus and Ebola virus, SARS-CoV-2

Bacterial antigens include, but are not limited to antigens of the following bacteria: Mycobacterium tuberculosis, Chlamydia, Neisseria gonorrhoeae, Shigella, Salmonella, Vibrio cholerae, Treponema pallidum, Pseudomonas, Bordetella pertussis, Brucella, Francisella tularensis, Helicobacter pylori, Leptospira interrogans, Legionella pneumophila, Yersinia pestis, Streptococcus (types A and B), Pneumococcus, Meningococcus, Haemophilus influenzae (type b), Toxoplasma gondii, Campylobacter, Moraxella catarrhalis, Klebsiella granulomatis and Actinomyces.

Fungal antigens include, but are not limited to antigens of the following fungal pathogens: Candida and Aspergillus, Cryptococcus, Histoplasma and Pneumocystis.

Parasitic antigens include, but are not limited to antigens of the following parasitic pathogens: Taenia, Flukes, Roundworms, Plasmodium, Amoeba, Giardia, Cryptosporidium, Schistosoma, Trichomonas, Trypanosoma and Trichinella.

In order to enhance expression of the antigen-second peptide partner prior to coupling, a leader sequence may be used. Those skilled in the art are aware of appropriate leader sequences to enhance expression. Such are exemplified herein.

### Pharmaceutical Composition and Use

The compositions of the invention may be incorporated into a vaccine or therapeutic composition. Suitably, a vaccine or immunogenic composition will comprise particles of the invention in an immunogenic dose.

A pharmaceutical composition may comprise a particle or composition in accordance with the invention provided with a pharmaceutically acceptable carrier. Suitable carriers are well known to those skilled in the art. In one embodiment a pharmaceutical composition comprises a buffer, excipient or carrier. Suitably a pharmaceutical composition may comprise suitable excipients and formulations to maintain stability of the composition. Suitably the formulation may comprise an adjuvant. In one embodiment, the formulation may comprise AddaVax™ or a similar squalene-based oil-in-water nano-emulsion with a formulation similar to MF59®. Other suitable adjuvants include liposome-based adjuvants such as Matrix M and AS01. Other suitable adjuvants include aluminium-based formulations such as Alhydrogel®. In one embodiment the formulation may comprise EDTA, for example at a concentration of 5 mM. Suitable excipients or formulations may depend on the properties of the particle or immunogenic composition; for example, the choice of expression system may affect the stability, glycosylation or folding of the proteins of the composition, which may in turn affect the optimal formulation of the composition. Methods of determination of a suitable excipient, formulation or adjuvant will be known to those skilled in the art.

### Vaccine

A vaccine is a preparation that comprises a fragment or entire entity against which it is possible to raise an immune response. It is an entity such as a protein, peptide, lipoprotein, glycoprotein or fragments thereof that are capable of inducing an immune response. For example, the vaccine may comprise micro-organisms or a part thereof capable of inducing an immune response against said micro-organism. A vaccine comprising an immunogenic adenoviral vector in accordance with the invention can be used against any pathogen for which the antigen displayed is crucial for the induction of an immune response. Further, the vaccine may comprise an immunogenic adenoviral vector in accordance with the invention displaying tumour related antigens. These tumour-related antigens may be modified self-proteins and the like. The vaccine may therefore raise an immune response to the tumour cells.

Such vaccine compositions (or other immunogenic) are formulated in a suitable delivery vehicle. Generally, doses for the immunogenic compositions are within the ranges defined for therapeutic compositions. Optionally, a vaccine composition of the invention may be formulated to contain other components, including, for example, adjuvants, stabilizers, pH adjusters, preservatives and the like. Such components are well known to those skilled in the art of vaccines. Examples of suitable adjuvants include, without limitation, liposomes, alum, monophosphoryl lipid A, and any biologically active factor, such as a cytokine, an interleukin, a chemokine and optimally combinations thereof.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, locally and/or using aerosol, injection, infusion, continuous infusion, localized perfusion bathing target cells directly or via a catheter and/or lavage.

Vaccines for the treatment or prevention of a disease as used herein includes but is not limited to urogenital cancers (such as prostate cancer, renal cell cancers, bladder cancers), gynaecological cancers (such as ovarian cancers, cervical cancers, endometrial cancers), lung cancer, gastrointestinal cancers (such as non-metastatic or metastatic colorectal cancers, pancreatic cancer, gastric cancer, oesophageal cancers, hepatocellular cancers, cholangiocellular cancers), head and neck cancer (e.g. head and neck squamous cell cancer), malignant glioblastoma, malignant mesothelioma, non-metastatic or metastatic breast cancer (e.g. hormone refractory metastatic breast cancer), malignant melanoma, Merkel Cell Carcinoma or bone and soft tissue sarcomas, and haematologic neoplasias, such as multiple myeloma, acute myelogenous leukaemia, chronic myelogenous leukaemia, myelodysplastic syndrome and acute lymphoblastic leukaemia. In a preferred embodiment, the disease is non-small cell lung cancer (NSCLC), breast cancer (e.g. hormone refractory metastatic breast cancer), head and neck cancer (e.g. head and neck squamous cell cancer), hormone sensitive or hormone refractory prostate cancer, colorectal cancer, ovarian cancer, hepatocellular cancer, renal cell cancer, soft tissue sarcoma, or small cell lung cancer.

The vaccine may be used to treat or prevent infection with any one of the disease-causing pathogens hereinbefore described.

### Ad-DogTag

The “Ad-DogTag” viral vector comprises the insertion of DogTag into surface loops of the hexon capsid protein enabling display of up to ^{˜}720 ligands/virion. Coupling of an antigen to hexon-DogTag has been achieved by the present inventors using SnoopTagJr-tagged antigens (using SnoopLigase as a catalyst) or directly via DogCatcher linked antigens. Previous technologies have only been capable of inserting small immunogenic T cell or B cell epitopes with a length of <100 amino acids into adenovirus hexon loops. The present invention demonstrates the coupling of peptides of 10-60 kDa to hexon, which has not previously been possible to achieve. This represents a big step forwards in the development of vaccines based upon adenovirus in particular.

### Ad-SpyCatcher

The “Ad-SpyCatcher” viral vector comprises the fusion of SpyCatcher onto the C-terminus of adenovirus minor capsid protein pIX. The recent invention was successful in modifying the pIX minor capsid protein without loss of viral infectivity.

### Ad-SnoopCatcher

The “Ad-SnoopCatcher” viral vector comprises the fusion of SnoopCatcher onto the C-terminus of adenovirus minor capsid protein pIX. The work here shows success in modifying the pIX minor capsid protein without loss of viral infectivity.

### Ad-DogCatcher

The “Ad-DogCatcher” viral vector comprises the fusion of DogCatcher onto the C-terminus of adenovirus minor capsid protein pIX. The work here shows success in modifying the pIX minor capsid protein without loss of viral infectivity.

### Ad-SnoopTagJr

The “Ad-SnoopTagJr” viral vector comprises the fusion of SnoopTagJr onto the C-terminus of adenovirus minor capsid protein pIX. The work here shows success in modifying the pIX minor capsid protein without loss of viral infectivity.

### Ad-SpyTag

The “Ad-SpyTag” viral vector comprises the fusion of SpyTag onto the C-terminus of adenovirus minor capsid protein pIX. The work here shows success in modifying the pIX minor capsid protein without loss of viral infectivity.

All references mentioned herein are incorporated by reference where permitted.

### EXAMPLES

Certain aspects and embodiments of the invention will now be illustrated by way of example and with reference to the figures described above.

### Examples—Materials and Methods

Generating a Bacterial Artificial Chromosome (BAC)-Derived Replication-Defective Molecular Clone of Ad5 Expressing GFP

Plasmid pAd-PL-DEST, an E1/E3-deleted (and therefore replication-defective) molecular clone of Ad5, was obtained from Invitrogen. An expression construct, consisting of an immediate early cytomegalovirus promoter (CMVp) driving expression of enhanced green fluorescent protein (EGFP), was cloned into shuttle vector pENTR4 (Invitrogen). The CMVp EGFP expression construct was then inserted into the Ad5 E1 locus using Invitrogen Gateway site-specific recombination (LR clonase) technology. BAC sequences from pBELOBAC11 (NEB) were amplified using forward (5′-TTAATTAAcgtcgaccaattctcatg) and reverse (5′-TTAATTAAgtcgacagcgacacacttg) primers to introduce PacI sites at either end of the BAC cassette. The entire Ad5(GFP) genome sequence was subsequently cloned into the BAC with PacI, to generate pBAC-Ad5(GFP).

Genetic Modification of PBAC-Ad5(GFP) to Insert Protein Superglue Technology into Viral Capsid Proteins Using BAC GalK Recombineering

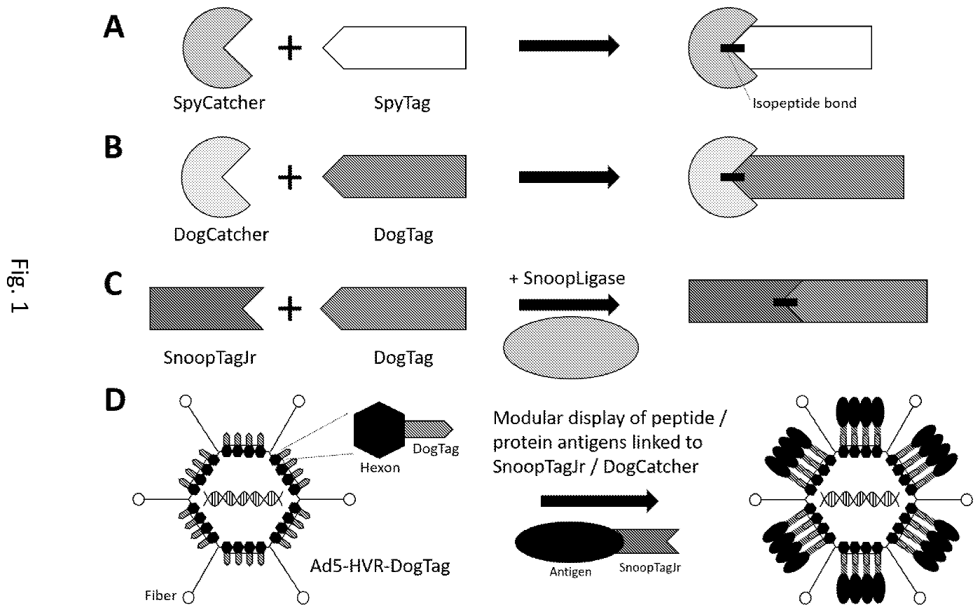

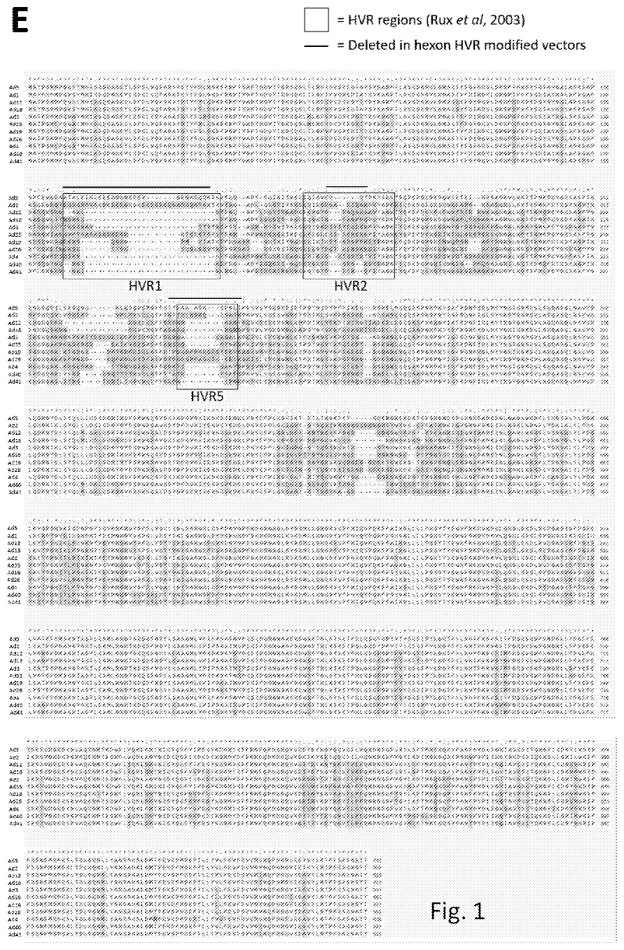

SW102, an E. coli strain required for GalK recombineering, was obtained from the National Cancer Institute, National Institutes of Health, USA. Modified from DH10B, SW102 cells contain A-Red-encoded recombination genes (exo, bet, gam) under the control of a temperature-sensitive repressor with a deleted galactokinase (GalK) gene (which is necessary for bacterial growth using galactose as the sole carbon source). The GalK recombineering system enables the GalK gene to be used for both positive and negative selection, and GalK recombineering was performed exactly as described in Warming et al, 2005 [Warming S, Costantino N, Court D L, Jenkins N A, Copeland N G. Simple and highly efficient BAC recombineering using galK selection. Nucleic Acids Res. 2005; 33(4):e36]. Insertion sites were created in hexon HVR loops (as described in FIG. 1E) or at the C-terminus of pIX by insertion of the Ga/K gene at these specific loci by recombineering followed by positive selection for the presence of Ga/K. Insertion of SpyTag/DogTag/SnoopTagJr/SpyCatcher/SnoopCatcher/DogCatcher sequences was performed by recombineering to replace Ga/K, and subsequent selection against the presence of the Ga/Kgene using 2-deoxygalactose.

Rescue of Recombinant Adenoviruses Incorporating Protein Superglue Technology

BAC DNA from recombinant adenovirus molecular clones was linearised with PacI to release left and right viral inverted terminal repeats (ITRs). Linearised DNA was transfected into E1-complementing Human Embryonic Kidney (HEK) 293A cells (Invitrogen) in 25 cm² flasks (T25) using Lipofectamine 2000 reagent (Invitrogen). After cytopathic effect (CPE) was observed, the cells were harvested, subjected to three cycles of freeze-thaw, and the virus amplified further in HEK293A cells. Upon infection of 10×150 cm² flasks (T150), virus was harvested from infected cells after 48 hours and purified by CsCl gradient ultracentrifugation according to standard protocols. Purified virus was dialysed against sucrose storage buffer (10 mM Tris-HCl, 7.5% w/v sucrose, pH 7.8) and stored at −80° C.

Estimation of Viral Particle Count for Purified Viral Vector Preparations The number of adenovirus particles in a purified preparation can be estimated by measuring viral DNA content by spectrophotometric absorption at 260 nm as described by Maizel et al, 1968. [J. Maizel, D. White, M. Scharff, The polypeptides of adenovirus: I. Evidence for multiple protein components in the virion and a comparison of types 2, 7A, and 12. Virology, Volume 36, Issue 1, September 1968, Pages 115-125]. Briefly, samples were diluted 1:10 in virus storage buffer containing 1% w/v sodium dodecyl sulphate (SDS) to release viral DNA from capsids and absorbance at 260 nm was measured using a spectrophotometer. An absorbance of 1.00 (AU, 1 cm path length) at 260 nm corresponds to 1.1×10¹² viral particles/mL.

Infectious Titration of Recombinant Adenoviruses in HEK293A Cells

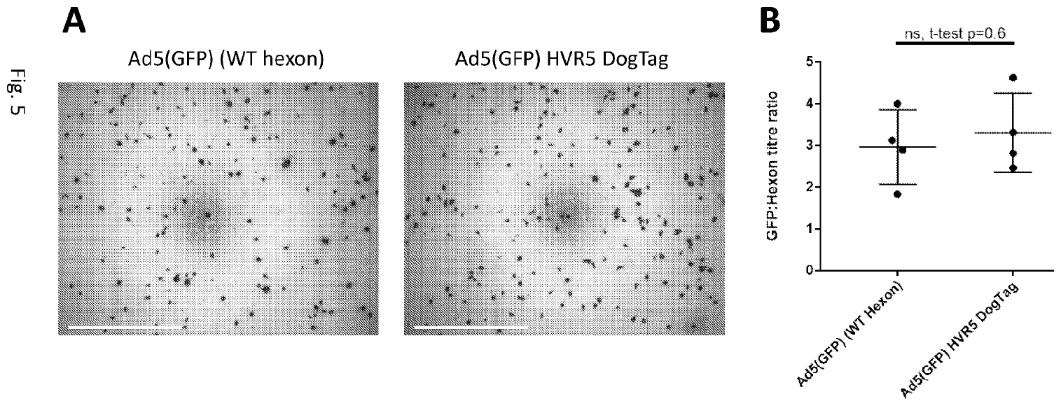

Infectious titre of vector preparations was assessed by single cell infectivity assay on HEK293A cells. For vectors expressing EGFP, infected HEK293 cells were visualised and enumerated directly by fluorescent microscopy. An alternative assay for vectors without a fluorescent marker, by immunostaining for expression of the hexon capsid protein, was also tested (FIG. 5). Tenfold serial dilutions of virus in complete media (Dulbecco's Modified Eagles Medium, plus 1×GlutaMAX and 10% v/v foetal bovine serum) were performed in sterile 96-well deep well plates. Two or three serial dilutions were performed per virus, and 50 μL of each dilution (10³ to 10¹⁰ dilution) was added per well of a 96-well plate containing adherent HEK293 cells at 80-90% confluency. For hexon immunostaining, 96-well plates pre coated with poly-L-Lysine were used to improve cell adhesion to the plate surface. Plates were incubated for 48 hours at 37° C., 5% CO₂. For titration of EGFP-expressing vectors, 48 hours post infection, single GFP-positive cells were enumerated by fluorescence microscopy, and an infectious titre was calculated in infectious units (ifu) per mL. For hexon immunostaining, media was aspirated from the cell monolayer and cells were fixed with ice-cold methanol. Plates were then washed three times in Dulbecco's phosphate buffered saline (PBS 1X, Gibco) before blocking for an hour with 3% w/v low-fat milk (Marvel). Detecting mouse monoclonal anti-hexon antibody (B025/AD51, Thermo-Fisher) was added at 1:1000 dilution in 1% w/v milk in PBS and incubated for an hour at 25° C. After incubation of the primary antibody, cells were washed a further three times with 1% w/v milk in PBS prior to addition of a secondary goat anti-mouse alkaline phosphatase (ALP) conjugated antibody (STAR117A, BioRad) at 1:1000 dilution in 1% w/v milk in PBS. After a further hour incubation, plates were washed five times in PBS prior to development. To develop, 100 μL of freshly prepared SIGMAfast BCIP/NBT solution (Sigma) was added to each well and plates incubated at 25° C. until the appearance of dark stained foci, representing single infected cells. P:I ratios for CsCl-purified vector preparations were calculated by dividing the estimated no. of viral particles per mL with the no. of infectious units (ifu) per mL.

Assessment of Coagulation Factor X-Mediated Vector Transduction of SKOV3 Cells

SKOV3 cells (human ovary adenocarcinoma) were obtained from Public Health England and cultured in McCoy's 5a media with 2 mM Glutamine and 15% v/v foetal bovine serum (complete McCoy's media). For the assay, GFP-expressing vectors were serially diluted (1:10 to 1:107) in serum free media. Human coagulation Factor X (FX) was added to diluted vectors at a final concentration of 8 μg/mL (control samples without addition of FX were included). Vector-FX mixtures were added to monolayers of SKOV3 cells (80-90% confluent) in 96-well plates, and incubated with cells for 2 h at 37° C. and 5% CO₂. After 2 h, vector-FX mixtures were replaced with complete McCoy's media, and plates incubated at 37° C., 5% CO₂ for a further 48 h. Infectivity was assessed after 48 h by enumeration of GFP-positive foci as described above.

Production of Protein and Peptide Ligands

SpyTag- and SnoopTagJr-fused peptide ligands were produced using solid-phase synthesis techniques by Insight Biotechnology at >95% purity. Peptides were quality control tested by HPLC and mass spectrometry.

DNA constructs for expression of polyhistidine-tagged recombinant DogCatcher-NANP fusion proteins were cloned into expression plasmid pET45(+) (EMD Millipore) for protein production in BL21(DE3) E. coli. (NEB). DNA sequences for DogCatcher and Plasmodium falciparum circumsporozoite protein (PfCSP) from the 3D7 strain of malaria were synthesised separately (GeneArt, Thermo Fisher), DNA fragments required for individual constructs amplified by PCR, and assembled in pET45(+) by restriction cloning. Recombinant proteins were purified using affinity Ni-NTA resin (Qiagen) according to a previously published protocol [SnoopLigase Catalyzes Peptide-Peptide Locking and Enables Solid-Phase Conjugate Isolation. Buldun C M, Jean J X, Bedford M R, Howarth M. J Am Chem Soc. 2018 Feb. 28; 140(8):3008-3018. doi: 10.1021/jacs.7b13237], dialysed into PBS, and stored at −80° C.

SpyCatcher (GenBank: AFD50637.1) and SpyTag-MBP (Addgene Plasmid #35050) were expressed in E. coli and purified by Ni-NTA exactly as described (Peptide tag forming a rapid covalent bond to a protein, through engineering a bacterial adhesin. Zakeri B, Fierer J O, Celik E, Chittock E C, Schwarz-Linek U, Moy V T, Howarth M. Proc Natl Acad Sci USA. 2012 Mar. 20; 109(12):E690-7. doi: 10.1073/pnas.1115485109).

Monovalent streptavidin (mSA) was expressed in E. coli, refolded from exclusion bodies and purified by ion-exchange chromatography exactly as described (Plug-and-play pairing via defined divalent streptavidins. Fairhead M, Krndija D, Lowe E D, Howarth M. J Mol Biol. 2014 Jan. 9; 426(1):199-214. doi: 10.1016/j.jmb.2013.09.016).

SnoopLigase (GenBank: AVD97783.1), SnoopTagJr-AffiHER2, and SUMO-DogTag (GenBank:MG867376) were expressed in E. coli and purified by Ni-NTA exactly as described (SnoopLigase Catalyzes Peptide-Peptide Locking and Enables Solid-Phase Conjugate Isolation. Buldun C M, Jean J X, Bedford M R, Howarth M. J Am Chem Soc. 2018 Feb. 28; 140(8):3008-3018. doi: 10.1021/jacs.7b13237).

DogCatcher (previously termed RrgACatcher in the patent “Methods and products for fusion protein synthesis” Howarth M, Veggiani G, Gayet R. 2015, United Kingdom Patent application WO2016193746A1) was expressed in E. coli and purified by Ni-NTA following standard protocols (Peptide tag forming a rapid covalent bond to a protein, through engineering a bacterial adhesin. Zakeri B, Fierer J O, Celik E, Chittock E C, Schwarz-Linek U, Moy V T, Howarth M. Proc Natl Acad Sci U S A. 2012 Mar. 20; 109(12):E690-7. doi: 10.1073/pnas.1115485109).

HCMV pentamer (with SpyTag-gH) is described in patent application PCT/GB2019/051245. Protein was expressed in suspension 293F cells by transient transfection using five separate plasmids (sequences provided). Protein was harvested, concentrated by tangential flow filtration, affinity purified by C-tag affinity resin (Thermo Fisher), and further purified by size exclusion chromatography on a Superdex 200 column (GE) using an AKTA chromatography system (GE).

DNA constructs for expression of SARS CoV2 Spike and RBD fusion proteins were cloned into mammalian protein expression plasmid pcDNA3.4. DNA sequences for SARS CoV2 Spike and RBD were synthesised separately (GeneArt, Thermo Fisher), and assembled in frame with SnoopTagJr, SpyCatcher, or SnoopCatcher by PCR amplification and restriction cloning. To facilitate secretion of RBD fusion constructs, a leader sequence was introduced at the N-terminus (for RBD-SnoopTagJr, RBD-SpyCatcher and RBD-SnoopCatcher the leader sequence from SARS CoV2 spike MFVFLVLLPLVSSQC was used, for SnoopCatcher-RBD the IgK leader sequence METDTLLLWVLLLWVPGSTGD was used). Spike-SnoopTagJr and RBD-SnoopTagJr proteins were expressed in suspension Expi293F cells, and SnoopCatcher-RBD, RBD-SnoopCatcher and RBD-SpyCatcher proteins were expressed in suspension ExpiCHO-S cells. Protein was harvested from culture supernatant, affinity purified using C-tag affinity resin (Thermo Fisher) using an AKTA chromatography system (GE), and dialysed into tris-buffered saline (TBS) pH 7.4.

Coupling Reactions

For in vitro assays, coupling reactions between SpyCatcher and SpyTag, DogCatcher and DogTag, SnoopCatcher and SnoopTagJr, and SnoopTagJr and DogTag (catalysed by SnoopLigase) partners were performed by co-incubation of spontaneously reacting components in a total volume of 20-25 μL, with individual components at concentrations described in the figure legends. Reactions between Ad5-HVR-DogTag and DogCatcher (including DogCatcher fusions), between Ad5-HVR-SpyTag or Ad5-pIX-SpyTag and SpyCatcher (including SpyCatcher fusions), between Ad5-pIX-SpyCatcher and SpyTag (including SpyTag fusions), between Ad5-pIX-SnoopCatcher and SnoopTagJr (including SnoopTagJr fusions), between Ad5-pIX-DogCatcher and DogTag (including DogTag fusions) and between Ad5-pIX-SnoopTagJr and SnoopCatcher (including SnoopCatcher fusions) were incubated for 16 h at 4° C. Reactions between Ad5-HVR-DogTag and SnoopTagJr (including SnoopTagJr fusions) catalysed by SnoopLigase were incubated for 48 h at 4° C. SnoopLigase catalysed reactions were performed in buffers containing a final concentration of 15% v/v glycerol and minimal salt to increase the efficiency of coupling.

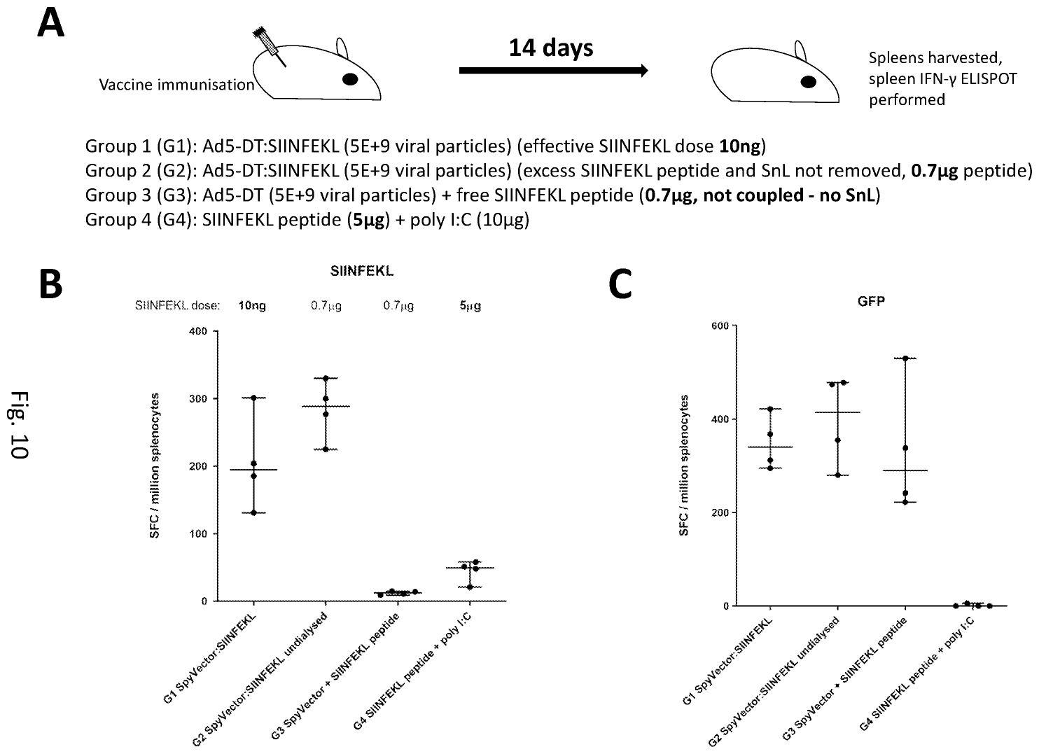

Peptide-decorated vector batches for immunisation studies were prepared by co-incubating 5E+11 viral particles of Ad5(GFP)-HVR5-DogTag with 35 μM SnoopTagJr-GGSSIINFEKL, 30 μM SnoopLigase, and 15% v/v glycerol in a total volume of 400 μL for 48 hours at 4° C. To remove excess peptide and SnoopLigase (FIG. 10A, Group 1) coupled vectors were dialysed into sucrose storage buffer using SpectraPor dialysis cassettes (100 kDa MWCO). No excess peptide was detectable post-dialysis on a coomassie-stained SDS-PAGE gel. Peptide-decorated vectors were stored at −80° C., endotoxin tested, and infectious titration of stored batches was repeated prior to immunisation.

Assessment of Coupling Efficiency by SDS-PAGE

Coupling reactions were performed as described above and stopped by addition of SDS loading buffer (BioRad, 31.5 mM Tris-HCl, pH 6.8, 10% glycerol, 1% SDS, 0.005% Bromophenol Blue, 300 mM DTT). Samples were boiled at 95° C. for 5 min and loaded on SDS-PAGE (NuPAGE 4-12% Bis-Tris, Invitrogen) gels. For direct gel shift assays to assess coupling of ligands to Ad5-HVR-DogTag or Ad5-HVR-SpyTag, proteins were resolved by SDS-PAGE (200V, 40-55 min) and visualized by Coomassie staining [16 h staining with Quick Coomassie (Generon), destained with water]. Coupling efficiency was assessed by comparing band intensities of uncoupled hexon-DogTag/hexon-SpyTag with hexon-DogTag:DogCatcher/hexon-SpyTag:SpyCatcher using Image J (For hexon-SpyTag:SpyCatcher coupling efficiency, % hexon coupled=band intensity of hexon-SpyTag:SpyCatcher divided by the sum of band intensities of hexon-SpyTag:SpyCatcher and uncoupled hexon-SpyTag, multiplied by 100).

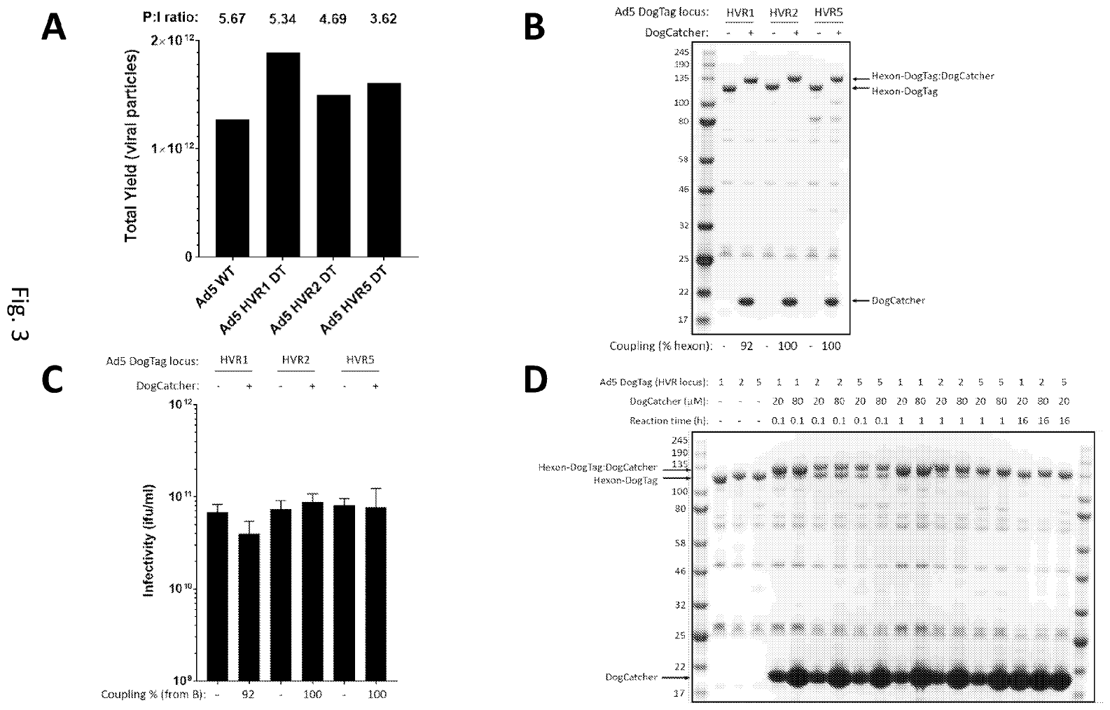

For assessment of coupling of Ad5-HVR-DogTag to SnoopTagJr fused peptides, a competition assay using DogCatcher (DC) protein was performed due to the fact that coupling of low molecular weight peptides could not be accurately assessed using the direct gel shift assay. After coupling, an excess of DogCatcher protein (30 μM) was added to the reaction and samples incubated at 4° C. for a further 24 hours. Since DC binds ^{˜}100% of free (uncoupled) hexon-DogTag molecules on the surface of Ad5 (see FIG. 3B) the proportion of hexon-DogTag coupled to SnoopTagJr-peptide can be estimated to be the proportion of hexon that does not undergo a gel shift of ^{˜}20 kDa (molecular weight of DogCatcher) on Coomassie-stained SDS-PAGE after incubation with DogCatcher.

An alternative assay to the DogCatcher competition assay, enabling assessment of SnoopTagJr-peptide coupling to hexon-DogTag by direct gel shift, exploited the high affinity biotin:streptavidin interaction (stable in room temperature SDS loading buffer). Biotinylated SnoopTagJr-peptide was incubated with SnoopLigase and Ad5-HVR-DogTag as described previously. After 48 hours, the coupling reaction was stopped by boiling in SDS loading buffer to denature all proteins and virion structures. Samples were briefly cooled on ice, before incubation with an excess of monovalent streptavidin (mSA, 2-fold excess over biotin peptide) for 30 min at 25° C. Samples were run on SDS-PAGE, transferred to nitrocellulose, and Western blotting performed using an anti-hexon primary mouse monoclonal antibody (clone 65H6, ThermoFisher). Migration of the hexon protein was visualised using a goat anti-mouse-alkaline phosphatase secondary (1:1000 dilution) followed by BCIP/NBT substrate for development (Sigma). A gel-shift (^{˜}50 kDa) was observed for hexon-DogTag protein coupled to SnoopTagJr-biotin by virtue of the interaction between mSA and biotin.

For assessment of coupling of protein ligands to Ad5-pIX-SpyCatcher, Ad5-pIX-SnoopCatcher, Ad5-pIX-DogCatcher, Ad5-pIX-SpyTag or Ad5-pIX-SnoopTagJr, samples were run on SDS-PAGE and then transferred to nitrocellulose for Western blotting. Protein species covalently coupled to pIX-fusions were detected using primary antibodies or mouse antisera and alkaline-phosphatase conjugated secondary antibodies as described in figure legends. Western blots were developed as described above.

Antibody Neutralisation Assay

For assessment of vector neutralisation by potent neutralising mouse monoclonal antibody (mAb) 9C12 (Developmental Studies Hybridoma Bank, University of Iowa), Ad5(GFP) vectors were incubated with serially diluted mAb 9C12 antibody at a 1:1 ratio in complete media for 1 hour at 37° C. The vector-antibody mix was then added to an 80% confluent monolayer of HEK293A cells in a 96-well plate format (cells were infected at a multiplicity of infection of 200 ifu/cell). Cells were incubated with the vector-antibody mix for 2 hours at 37° C. 5% CO₂, before the mix was replaced with fresh media and the plates returned to 37° C. 5% CO₂ for a further 24 h. After 24 h, GFP expression within HEK293A cells was used as a readout of vector infectivity; bulk fluorescence was measured on a fluorimeter (Tecan) using an excitation wavelength of 395 nm and emission wavelength of 509 nm.

For assessment of vector neutralisation by Ad5-positive serum, serum samples were obtained by immunising mice with 1E+8 ifu of an Ad5 vector expressing ovalbumin (vector had an unmodified hexon). Serum was harvested two-weeks post immunization, stored at −80° C., and then serially diluted for the neutralisation assay (two-fold dilutions were prepared from 1:8 to 1:1024 in complete media, to give a final range of 1:16 to 1:2048 on cell monolayers). Diluted serum was incubated with Ad5(GFP) vectors, the mix incubated on HEK293 cells and bulk GFP fluorescence read 24 h later exactly as described above.

Mouse Immunisations

All mouse procedures were performed in accordance with the terms of the UK Animals (Scientific Procedures) Act Project Licence (PA7D20B85) and approved by the Oxford University Ethical Review Body. Female C57BL/6 mice (6 weeks of age, Charles River), housed in specific-pathogen free environments, were immunised intramuscularly by injection of 50 μL of vaccine formulated in endotoxin-free PBS (Gibco) into both hind limbs of each animal (100 μL total). Adenoviral vectors were administered at a dose of 5E+9 viral particles, peptides administered at a dose of 5 μg, and poly I:C (InvivoGen) administered at a dose of 10 μg. Endotoxin dose was <1 EU per mouse. Experiments were performed at Biomedical Services, University of Oxford, and completed two-weeks post-immunisation.

Ex-Vivo IFN-Gamma ELISPOT

Spleen ex vivo interferon-gamma (IFN-γ) ELISpot was performed according to standard protocols as described previously [Larsen K C, Spencer A J, Goodman A L, Gilchrist A, Furze J, Rollier C S, Kiss-Toth E, Gilbert S C, Bregu M, Soilleux E J, Hill A V, Wyllie D H, Expression of tak1 and tram induces synergistic pro-inflammatory signalling and adjuvants DNA vaccines. Vaccine. 2009 Sep. 18; 27(41):5589-98]. To measure antigen specific responses, cells were re-stimulated for 18-20 hours with peptides at a final concentration of 5 μg/mL. To measure SIINFEKL-specific responses, SIINFEKL peptide (Cambridge Bioscience) was used. For GFP-specific responses, EGFP peptide DTLVNRIEL (EGFP₁₁₈₋₁₂₆) (synthesised by Insight Biotechnology) was used. Spot forming cells (SFC) were measured using an automated ELISpot reader system (AID).

Modified adenovirus sequences:
Ad5-HVR-SpyTag sequences
Ad5-HVR1-SpyTag Hexon sequence
Amino acid (SEQ ID NO: 1):
MATPSMMPQWSYMHISGQDASEYLSPGLVQFARATETYFSLNNKFRNPTVAPTHDVTTDRSQRLTLRFIPVDREDTAYSYKARFTLA
VGDNRVLDMASTYFDIRGVLDRGPTFKPYSGTAYNALAPKGAPNPCEWDEAGSGGSGAHIVMVDAYKPTKGSGGSGTHVFGQAPYSG
INITKEGIQIGVEGQTPKYADKTFQPEPQIGESQWYETEINHAAGRVLKKTTPMKPCYGSYAKPTNENGGQGILVKQQNGKLESQVE
MQFFSTTEATAGNGDNLTPKVVLYSEDVDIETPDTHISYMPTIKEGNSRELMGQQSMPNRPNYIAFRDNFIGLMYYNSTGNMGVLAG
QASQLNAVVDLQDRNTELSYQLLLDSIGDRTRYFSMWNQAVDSYDPDVRIIENHGTEDELPNYCFPLGGVINTETLTKVKPKTGQEN
GWEKDATEFSDKNEIRVGNNFAMEINLNANLWRNFLYSNIALYLPDKLKYSPSNVKISDNPNTYDYMNKRVVAPGLVDCYINLGARW
SLDYMDNVNPFNHHRNAGLRYRSMLLGNGRYVPFHIQVPQKFFAIKNLLLLPGSYTYEWNFRKDVNMVLQSSLGNDLRVDGASIKFD
SICLYATFFPMAHNTASTLEAMLRNDTNDQSFNDYLSAANMLYPIPANATNVPISIPSRNWAAFRGWAFTRLKTKETPSLGSGYDPY
YTYSGSIPYLDGTFYLNHTFKKVAITFDSSVSWPGNDRLLTPNEFEIKRSVDGEGYNVAQCNMTKDWFLVQMLANYNIGYQGFYIPE
SYKDRMYSFFRNFQPMSRQVVDDTKYKDYQQVGILHQHNNSGFVGYLAPTMREGQAYPANFPYPLIGKTAVDSITQKKFLCDRTLWR
IPFSSNFMSMGALTDLGQNLLYANSAHALDMTFEVDPMDEPTLLYVLFEVFDVVRVHRPHRGVIETVYLRTPFSAGNATT
DNA (incl. STOP) (SEQ ID NO: 2):
ATGGCTACCCCTTCGATGATGCCGCAGTGGTCTTACATGCACATCTCGGGCCAGGACGCCTCGGAGTACCTGAGCCCCGGGCTGGTG
CAGTTTGCCCGCGCCACCGAGACGTACTTCAGCCTGAATAACAAGTTTAGAAACCCCACGGTGGCGCCTACGCACGACGTGACCACA
GACCGGTCCCAGCGTTTGACGCTGCGGTTCATCCCTGTGGACCGTGAGGATACTGCGTACTCGTACAAGGCGCGGTTCACCCTAGCT
GTGGGTGATAACCGTGTGCTGGACATGGCTTCCACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAATGGGATGAAGCTGGCAGCGGAGGATCCGGCGCC
CATATCGTGATGGTGGACGCCTACAAGCCTACCAAAGGCTCTGGCGGAAGCGGCACTCACGTATTTGGGCAGGCGCCTTATTCTGGT
ATAAATATTACAAAGGAGGGTATTCAAATAGGTGTCGAAGGTCAAACACCTAAATATGCCGATAAAACATTTCAACCTGAACCTCAA
ATAGGAGAATCTCAGTGGTACGAAACTGAAATTAATCATGCAGCTGGGAGAGTCCTTAAAAAGACTACCCCAATGAAACCATGTTAC
GGTTCATATGCAAAACCCACAAATGAAAATGGAGGGCAAGGCATTCTTGTAAAGCAACAAAATGGAAAGCTAGAAAGTCAAGTGGAA
ATGCAATTTTTCTCAACTACTGAGGCGACCGCAGGCAATGGTGATAACTTGACTCCTAAAGTGGTATTGTACAGTGAAGATGTAGAT
ATAGAAACCCCAGACACTCATATTTCTTACATGCCCACTATTAAGGAAGGTAACTCACGAGAACTAATGGGCCAACAATCTATGCCC
AACAGGCCTAATTACATTGCTTTTAGGGACAATTTTATTGGTCTAATGTATTACAACAGCACGGGTAATATGGGTGTTCTGGCGGGC
CAAGCATCGCAGTTGAATGCTGTTGTAGATTTGCAAGACAGAAACACAGAGCTTTCATACCAGCTTTTGCTTGATTCCATTGGTGAT
AGAACCAGGTACTTTTCTATGTGGAATCAGGCTGTTGACAGCTATGATCCAGATGTTAGAATTATTGAAAATCATGGAACTGAAGAT
GAACTTCCAAATTACTGCTTTCCACTGGGAGGTGTGATTAATACAGAGACTCTTACCAAGGTAAAACCTAAAACAGGTCAGGAAAAT
GGATGGGAAAAAGATGCTACAGAATTTTCAGATAAAAATGAAATAAGAGTTGGAAATAATTTTGCCATGGAAATCAATCTAAATGCC
AACCTGTGGAGAAATTTCCTGTACTCCAACATAGCGCTGTATTTGCCCGACAAGCTAAAGTACAGTCCTTCCAACGTAAAAATTTCT
GATAACCCAAACACCTACGACTACATGAACAAGCGAGTGGTGGCTCCCGGGTTAGTGGACTGCTACATTAACCTTGGAGCACGCTGG
TCCCTTGACTATATGGACAACGTCAACCCATTTAACCACCACCGCAATGCTGGCCTGCGCTACCGCTCAATGTTGCTGGGCAATGGT
CGCTATGTGCCCTTCCACATCCAGGTGCCTCAGAAGTTCTTTGCCATTAAAAACCTCCTTCTCCTGCCGGGCTCATACACCTACGAG
TGGAACTTCAGGAAGGATGTTAACATGGTTCTGCAGAGCTCCCTAGGAAATGACCTAAGGGTTGACGGAGCCAGCATTAAGTTTGAT
AGCATTTGCCTTTACGCCACCTTCTTCCCCATGGCCCACAACACCGCCTCCACGCTTGAGGCCATGCTTAGAAACGACACCAACGAC
CAGTCCTTTAACGACTATCTCTCCGCCGCCAACATGCTCTACCCTATACCCGCCAACGCTACCAACGTGCCCATATCCATCCCCTCC
CGCAACTGGGCGGCTTTCCGCGGCTGGGCCTTCACGCGCCTTAAGACTAAGGAAACCCCATCACTGGGCTCGGGCTACGACCCTTAT
TACACCTACTCTGGCTCTATACCCTACCTAGATGGAACCTTTTACCTCAACCACACCTTTAAGAAGGTGGCCATTACCTTTGACTCT
TCTGTCAGCTGGCCTGGCAATGACCGCCTGCTTACCCCCAACGAGTTTGAAATTAAGCGCTCAGTTGACGGGGAGGGTTACAACGTT
GCCCAGTGTAACATGACCAAAGACTGGTTCCTGGTACAAATGCTAGCTAACTACAACATTGGCTACCAGGGCTTCTATATCCCAGAG
AGCTACAAGGACCGCATGTACTCCTTCTTTAGAAACTTCCAGCCCATGAGCCGTCAGGTGGTGGATGATACTAAATACAAGGACTAC
CAACAGGTGGGCATCCTACACCAACACAACAACTCTGGATTTGTTGGCTACCTTGCCCCCACCATGCGCGAAGGACAGGCCTACCCT
GCTAACTTCCCCTATCCGCTTATAGGCAAGACCGCAGTTGACAGCATTACCCAGAAAAAGTTTCTTTGCGATCGCACCCTTTGGCGC
ATCCCATTCTCCAGTAACTTTATGTCCATGGGCGCACTCACAGACCTGGGCCAAAACCTTCTCTACGCCAACTCCGCCCACGCGCTA
GACATGACTTTTGAGGTGGATCCCATGGACGAGCCCACCCTTCTTTATGTTTTGTTTGAAGTCTTTGACGTGGTCCGTGTGCACCGG
CCGCACCGCGGCGTCATCGAAACCGTGTACCTGCGCACGCCCTTCTCGGCCGGCAACGCCACAACATAA
Ad5-HVR2-SpyTag Hexon sequence
Amino acid (SEQ ID NO: 3):
MATPSMMPQWSYMHISGQDASEYLSPGLVQFARATETYFSLNNKFRNPTVAPTHDVTTDRSQRLTLRFIPVDREDTAYSYKARFTLA
VGDNRVLDMASTYFDIRGVLDRGPTFKPYSGTAYNALAPKGAPNPCEWDEAATALEINLEEEDDDNEDEVDEQAEQQKTHVFGQAPY
SGINITKEGIQIGVGSGGSGAHIVMVDAYKPTKGSGGSGPKYADKTFQPEPQIGESQWYETEINHAAGRVLKKTTPMKPCYGSYAKP
TNENGGQGILVKQQNGKLESQVEMQFFSTTEATAGNGDNLTPKVVLYSEDVDIETPDTHISYMPTIKEGNSRELMGQQSMPNRPNYI
AFRDNFIGLMYYNSTGNMGVLAGQASQLNAVVDLQDRNTELSYQLLLDSIGDRTRYFSMWNQAVDSYDPDVRIIENHGTEDELPNYC
FPLGGVINTETLTKVKPKTGQENGWEKDATEFSDKNEIRVGNNFAMEINLNANLWRNFLYSNIALYLPDKLKYSPSNVKISDNPNTY
DYMNKRVVAPGLVDCYINLGARWSLDYMDNVNPFNHHRNAGLRYRSMLLGNGRYVPFHIQVPQKFFAIKNLLLLPGSYTYEWNFRKD
VNMVLQSSLGNDLRVDGASIKFDSICLYATFFPMAHNTASTLEAMLRNDTNDQSFNDYLSAANMLYPIPANATNVPISIPSRNWAAF
RGWAFTRLKTKETPSLGSGYDPYYTYSGSIPYLDGTFYLNHTFKKVAITFDSSVSWPGNDRLLTPNEFEIKRSVDGEGYNVAQCNMT
KDWFLVQMLANYNIGYQGFYIPESYKDRMYSFFRNFQPMSRQVVDDTKYKDYQQVGILHQHNNSGFVGYLAPTMREGQAYPANFPYP
LIGKTAVDSITQKKFLCDRTLWRIPFSSNFMSMGALTDLGQNLLYANSAHALDMTFEVDPMDEPTLLYVLFEVFDVVRVHRPHRGVI
ETVYLRTPFSAGNATT
DNA (incl. STOP) (SEQ ID NO: 4):
ATGGCTACCCCTTCGATGATGCCGCAGTGGTCTTACATGCACATCTCGGGCCAGGACGCCTCGGAGTACCTGAGCCCCGGGCTGGTG
CAGTTTGCCCGCGCCACCGAGACGTACTTCAGCCTGAATAACAAGTTTAGAAACCCCACGGTGGCGCCTACGCACGACGTGACCACA
GACCGGTCCCAGCGTTTGACGCTGCGGTTCATCCCTGTGGACCGTGAGGATACTGCGTACTCGTACAAGGCGCGGTTCACCCTAGCT
GTGGGTGATAACCGTGTGCTGGACATGGCTTCCACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAATGGGATGAAGCTGCTACTGCTCTTGAAATAAAC
CTAGAAGAAGAGGACGATGACAACGAAGACGAAGTAGACGAGCAAGCTGAGCAGCAAAAAACTCACGTATTTGGGCAGGCGCCTTAT
TCTGGTATAAATATTACAAAGGAGGGTATTCAAATAGGTGTCGGCAGCGGAGGATCCGGCGCCCATATCGTGATGGTGGACGCCTAC
AAGCCTACCAAAGGCTCTGGCGGAAGCGGCCCTAAATATGCCGATAAAACATTTCAACCTGAACCTCAAATAGGAGAATCTCAGTGG
TACGAAACTGAAATTAATCATGCAGCTGGGAGAGTCCTTAAAAAGACTACCCCAATGAAACCATGTTACGGTTCATATGCAAAACCC
ACAAATGAAAATGGAGGGCAAGGCATTCTTGTAAAGCAACAAAATGGAAAGCTAGAAAGTCAAGTGGAAATGCAATTTTTCTCAACT
ACTGAGGCGACCGCAGGCAATGGTGATAACTTGACTCCTAAAGTGGTATTGTACAGTGAAGATGTAGATATAGAAACCCCAGACACT
CATATTTCTTACATGCCCACTATTAAGGAAGGTAACTCACGAGAACTAATGGGCCAACAATCTATGCCCAACAGGCCTAATTACATT
GCTTTTAGGGACAATTTTATTGGTCTAATGTATTACAACAGCACGGGTAATATGGGTGTTCTGGCGGGCCAAGCATCGCAGTTGAAT
GCTGTTGTAGATTTGCAAGACAGAAACACAGAGCTTTCATACCAGCTTTTGCTTGATTCCATTGGTGATAGAACCAGGTACTTTTCT
ATGTGGAATCAGGCTGTTGACAGCTATGATCCAGATGTTAGAATTATTGAAAATCATGGAACTGAAGATGAACTTCCAAATTACTGC
TTTCCACTGGGAGGTGTGATTAATACAGAGACTCTTACCAAGGTAAAACCTAAAACAGGTCAGGAAAATGGATGGGAAAAAGATGCT
ACAGAATTTTCAGATAAAAATGAAATAAGAGTTGGAAATAATTTTGCCATGGAAATCAATCTAAATGCCAACCTGTGGAGAAATTTC
CTGTACTCCAACATAGCGCTGTATTTGCCCGACAAGCTAAAGTACAGTCCTTCCAACGTAAAAATTTCTGATAACCCAAACACCTAC
GACTACATGAACAAGCGAGTGGTGGCTCCCGGGTTAGTGGACTGCTACATTAACCTTGGAGCACGCTGGTCCCTTGACTATATGGAC
AACGTCAACCCATTTAACCACCACCGCAATGCTGGCCTGCGCTACCGCTCAATGTTGCTGGGCAATGGTCGCTATGTGCCCTTCCAC
ATCCAGGTGCCTCAGAAGTTCTTTGCCATTAAAAACCTCCTTCTCCTGCCGGGCTCATACACCTACGAGTGGAACTTCAGGAAGGAT
GTTAACATGGTTCTGCAGAGCTCCCTAGGAAATGACCTAAGGGTTGACGGAGCCAGCATTAAGTTTGATAGCATTTGCCTTTACGCC
ACCTTCTTCCCCATGGCCCACAACACCGCCTCCACGCTTGAGGCCATGCTTAGAAACGACACCAACGACCAGTCCTTTAACGACTAT
CTCTCCGCCGCCAACATGCTCTACCCTATACCCGCCAACGCTACCAACGTGCCCATATCCATCCCCTCCCGCAACTGGGCGGCTTTC
CGCGGCTGGGCCTTCACGCGCCTTAAGACTAAGGAAACCCCATCACTGGGCTCGGGCTACGACCCTTATTACACCTACTCTGGCTCT
ATACCCTACCTAGATGGAACCTTTTACCTCAACCACACCTTTAAGAAGGTGGCCATTACCTTTGACTCTTCTGTCAGCTGGCCTGGC
AATGACCGCCTGCTTACCCCCAACGAGTTTGAAATTAAGCGCTCAGTTGACGGGGAGGGTTACAACGTTGCCCAGTGTAACATGACC
AAAGACTGGTTCCTGGTACAAATGCTAGCTAACTACAACATTGGCTACCAGGGCTTCTATATCCCAGAGAGCTACAAGGACCGCATG
TACTCCTTCTTTAGAAACTTCCAGCCCATGAGCCGTCAGGTGGTGGATGATACTAAATACAAGGACTACCAACAGGTGGGCATCCTA
CACCAACACAACAACTCTGGATTTGTTGGCTACCTTGCCCCCACCATGCGCGAAGGACAGGCCTACCCTGCTAACTTCCCCTATCCG
CTTATAGGCAAGACCGCAGTTGACAGCATTACCCAGAAAAAGTTTCTTTGCGATCGCACCCTTTGGCGCATCCCATTCTCCAGTAAC
TTTATGTCCATGGGCGCACTCACAGACCTGGGCCAAAACCTTCTCTACGCCAACTCCGCCCACGCGCTAGACATGACTTTTGAGGTG
GATCCCATGGACGAGCCCACCCTTCTTTATGTTTTGTTTGAAGTCTTTGACGTGGTCCGTGTGCACCGGCCGCACCGCGGCGTCATC
GAAACCGTGTACCTGCGCACGCCCTTCTCGGCCGGCAACGCCACAACATAA
Ad5-HVR5-SpyTag Hexon sequence
Amino acid (SEQ ID NO: 5):
MATPSMMPQWSYMHISGQDASEYLSPGLVQFARATETYFSLNNKFRNPTVAPTHDVTTDRSQRLTLRFIPVDREDTAYSYKARFTLA
VGDNRVLDMASTYFDIRGVLDRGPTFKPYSGTAYNALAPKGAPNPCEWDEAATALEINLEEEDDDNEDEVDEQAEQQKTHVFGQAPY
SGINITKEGIQIGVEGQTPKYADKTFQPEPQIGESQWYETEINHAAGRVLKKTTPMKPCYGSYAKPTNENGGQGILVKQQNGKLESQ
VEMQFFSGSGGSGAHIVMVDAYKPTKGSGGSGPKVVLYSEDVDIETPDTHISYMPTIKEGNSRELMGQQSMPNRPNYIAFRDNFIGL
MYYNSTGNMGVLAGQASQLNAVVDLQDRNTELSYQLLLDSIGDRTRYFSMWNQAVDSYDPDVRIIENHGTEDELPNYCFPLGGVINT
ETLTKVKPKTGQENGWEKDATEFSDKNEIRVGNNFAMEINLNANLWRNFLYSNIALYLPDKLKYSPSNVKISDNPNTYDYMNKRVVA
PGLVDCYINLGARWSLDYMDNVNPFNHHRNAGLRYRSMLLGNGRYVPFHIQVPQKFFAIKNLLLLPGSYTYEWNFRKDVNMVLQSSL
GNDLRVDGASIKFDSICLYATFFPMAHNTASTLEAMLRNDTNDQSFNDYLSAANMLYPIPANATNVPISIPSRNWAAFRGWAFTRLK
TKETPSLGSGYDPYYTYSGSIPYLDGTFYLNHTFKKVAITFDSSVSWPGNDRLLTPNEFEIKRSVDGEGYNVAQCNMTKDWFLVQML
ANYNIGYQGFYIPESYKDRMYSFFRNFQPMSRQVVDDTKYKDYQQVGILHQHNNSGFVGYLAPTMREGQAYPANFPYPLIGKTAVDS
ITQKKFLCDRTLWRIPFSSNFMSMGALTDLGQNLLYANSAHALDMTFEVDPMDEPTLLYVLFEVFDVVRVHRPHRGVIETVYLRTPF
SAGNATT
DNA (incl. STOP) (SEQ ID NO: 6):
ATGGCTACCCCTTCGATGATGCCGCAGTGGTCTTACATGCACATCTCGGGCCAGGACGCCTCGGAGTACCTGAGCCCCGGGCTGGTG
CAGTTTGCCCGCGCCACCGAGACGTACTTCAGCCTGAATAACAAGTTTAGAAACCCCACGGTGGCGCCTACGCACGACGTGACCACA
GACCGGTCCCAGCGTTTGACGCTGCGGTTCATCCCTGTGGACCGTGAGGATACTGCGTACTCGTACAAGGCGCGGTTCACCCTAGCT
GTGGGTGATAACCGTGTGCTGGACATGGCTTCCACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAATGGGATGAAGCTGCTACTGCTCTTGAAATAAAC
CTAGAAGAAGAGGACGATGACAACGAAGACGAAGTAGACGAGCAAGCTGAGCAGCAAAAAACTCACGTATTTGGGCAGGCGCCTTAT
TCTGGTATAAATATTACAAAGGAGGGTATTCAAATAGGTGTCGAAGGTCAAACACCTAAATATGCCGATAAAACATTTCAACCTGAA
CCTCAAATAGGAGAATCTCAGTGGTACGAAACTGAAATTAATCATGCAGCTGGGAGAGTCCTTAAAAAGACTACCCCAATGAAACCA
TGTTACGGTTCATATGCAAAACCCACAAATGAAAATGGAGGGCAAGGCATTCTTGTAAAGCAACAAAATGGAAAGCTAGAAAGTCAA
GTGGAAATGCAATTTTTCTCAGGCAGCGGAGGATCCGGCGCCCATATCGTGATGGTGGACGCCTACAAGCCTACCAAAGGCTCTGGC
GGAAGCGGCCCTAAAGTGGTATTGTACAGTGAAGATGTAGATATAGAAACCCCAGACACTCATATTTCTTACATGCCCACTATTAAG
GAAGGTAACTCACGAGAACTAATGGGCCAACAATCTATGCCCAACAGGCCTAATTACATTGCTTTTAGGGACAATTTTATTGGTCTA
ATGTATTACAACAGCACGGGTAATATGGGTGTTCTGGCGGGCCAAGCATCGCAGTTGAATGCTGTTGTAGATTTGCAAGACAGAAAC
ACAGAGCTTTCATACCAGCTTTTGCTTGATTCCATTGGTGATAGAACCAGGTACTTTTCTATGTGGAATCAGGCTGTTGACAGCTAT
GATCCAGATGTTAGAATTATTGAAAATCATGGAACTGAAGATGAACTTCCAAATTACTGCTTTCCACTGGGAGGTGTGATTAATACA
GAGACTCTTACCAAGGTAAAACCTAAAACAGGTCAGGAAAATGGATGGGAAAAAGATGCTACAGAATTTTCAGATAAAAATGAAATA
AGAGTTGGAAATAATTTTGCCATGGAAATCAATCTAAATGCCAACCTGTGGAGAAATTTCCTGTACTCCAACATAGCGCTGTATTTG
CCCGACAAGCTAAAGTACAGTCCTTCCAACGTAAAAATTTCTGATAACCCAAACACCTACGACTACATGAACAAGCGAGTGGTGGCT
CCCGGGTTAGTGGACTGCTACATTAACCTTGGAGCACGCTGGTCCCTTGACTATATGGACAACGTCAACCCATTTAACCACCACCGC
AATGCTGGCCTGCGCTACCGCTCAATGTTGCTGGGCAATGGTCGCTATGTGCCCTTCCACATCCAGGTGCCTCAGAAGTTCTTTGCC
ATTAAAAACCTCCTTCTCCTGCCGGGCTCATACACCTACGAGTGGAACTTCAGGAAGGATGTTAACATGGTTCTGCAGAGCTCCCTA
GGAAATGACCTAAGGGTTGACGGAGCCAGCATTAAGTTTGATAGCATTTGCCTTTACGCCACCTTCTTCCCCATGGCCCACAACACC
GCCTCCACGCTTGAGGCCATGCTTAGAAACGACACCAACGACCAGTCCTTTAACGACTATCTCTCCGCCGCCAACATGCTCTACCCT
ATACCCGCCAACGCTACCAACGTGCCCATATCCATCCCCTCCCGCAACTGGGCGGCTTTCCGCGGCTGGGCCTTCACGCGCCTTAAG
ACTAAGGAAACCCCATCACTGGGCTCGGGCTACGACCCTTATTACACCTACTCTGGCTCTATACCCTACCTAGATGGAACCTTTTAC
CTCAACCACACCTTTAAGAAGGTGGCCATTACCTTTGACTCTTCTGTCAGCTGGCCTGGCAATGACCGCCTGCTTACCCCCAACGAG
TTTGAAATTAAGCGCTCAGTTGACGGGGAGGGTTACAACGTTGCCCAGTGTAACATGACCAAAGACTGGTTCCTGGTACAAATGCTA
GCTAACTACAACATTGGCTACCAGGGCTTCTATATCCCAGAGAGCTACAAGGACCGCATGTACTCCTTCTTTAGAAACTTCCAGCCC
ATGAGCCGTCAGGTGGTGGATGATACTAAATACAAGGACTACCAACAGGTGGGCATCCTACACCAACACAACAACTCTGGATTTGTT
GGCTACCTTGCCCCCACCATGCGCGAAGGACAGGCCTACCCTGCTAACTTCCCCTATCCGCTTATAGGCAAGACCGCAGTTGACAGC
ATTACCCAGAAAAAGTTTCTTTGCGATCGCACCCTTTGGCGCATCCCATTCTCCAGTAACTTTATGTCCATGGGCGCACTCACAGAC
CTGGGCCAAAACCTTCTCTACGCCAACTCCGCCCACGCGCTAGACATGACTTTTGAGGTGGATCCCATGGACGAGCCCACCCTTCTT
TATGTTTTGTTTGAAGTCTTTGACGTGGTCCGTGTGCACCGGCCGCACCGCGGCGTCATCGAAACCGTGTACCTGCGCACGCCCTTC
TCGGCCGGCAACGCCACAACATAA
Ad5-HVR-DogTag sequences
Ad5-HVR1-DogTag Hexon sequence
Amino acid (SEQ ID NO: 7):
MATPSMMPQWSYMHISGQDASEYLSPGLVQFARATETYFSLNNKFRNPTVAPTHDVTTDRSQRLTLRFIPVDREDTAYSYKARFTLA
VGDNRVLDMASTYFDIRGVLDRGPTFKPYSGTAYNALAPKGAPNPCEWDEAGSGGSGDIPATYEFTDGKHYITNEPIPPKGSGGSGT
HVFGQAPYSGINITKEGIQIGVEGQTPKYADKTFQPEPQIGESQWYETEINHAAGRVLKKTTPMKPCYGSYAKPTNENGGQGILVKQ
QNGKLESQVEMQFFSTTEATAGNGDNLTPKVVLYSEDVDIETPDTHISYMPTIKEGNSRELMGQQSMPNRPNYIAFRDNFIGLMYYN
STGNMGVLAGQASQLNAVVDLQDRNTELSYQLLLDSIGDRTRYFSMWNQAVDSYDPDVRIIENHGTEDELPNYCFPLGGVINTETLT
KVKPKTGQENGWEKDATEFSDKNEIRVGNNFAMEINLNANLWRNFLYSNIALYLPDKLKYSPSNVKISDNPNTYDYMNKRVVAPGLV
DCYINLGARWSLDYMDNVNPFNHHRNAGLRYRSMLLGNGRYVPFHIQVPQKFFAIKNLLLLPGSYTYEWNFRKDVNMVLQSSLGNDL
RVDGASIKFDSICLYATFFPMAHNTASTLEAMLRNDTNDQSFNDYLSAANMLYPIPANATNVPISIPSRNWAAFRGWAFTRLKTKET
PSLGSGYDPYYTYSGSIPYLDGTFYLNHTFKKVAITFDSSVSWPGNDRLLTPNEFEIKRSVDGEGYNVAQCNMTKDWFLVQMLANYN
IGYQGFYIPESYKDRMYSFFRNFQPMSRQVVDDTKYKDYQQVGILHQHNNSGFVGYLAPTMREGQAYPANFPYPLIGKTAVDSITQK
KFLCDRTLWRIPFSSNFMSMGALTDLGQNLLYANSAHALDMTFEVDPMDEPTLLYVLFEVFDVVRVHRPHRGVIETVYLRTPFSAGN
ATT
DNA (incl. STOP) (SEQ ID NO: 8):
ATGGCTACCCCTTCGATGATGCCGCAGTGGTCTTACATGCACATCTCGGGCCAGGACGCCTCGGAGTACCTGAGCCCCGGGCTGGTG
CAGTTTGCCCGCGCCACCGAGACGTACTTCAGCCTGAATAACAAGTTTAGAAACCCCACGGTGGCGCCTACGCACGACGTGACCACA
GACCGGTCCCAGCGTTTGACGCTGCGGTTCATCCCTGTGGACCGTGAGGATACTGCGTACTCGTACAAGGCGCGGTTCACCCTAGCT
GTGGGTGATAACCGTGTGCTGGACATGGCTTCCACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAATGGGATGAAGCTGGCAGCGGAGGATCCGGCgat
attccggctacatatgaatttaccgatggtaaacattatatcaccaatgaaccgataccgccgaaaGGCTCTGGCGGAAGCGGCACT
CACGTATTTGGGCAGGCGCCTTATTCTGGTATAAATATTACAAAGGAGGGTATTCAAATAGGTGTCGAAGGTCAAACACCTAAATAT
GCCGATAAAACATTTCAACCTGAACCTCAAATAGGAGAATCTCAGTGGTACGAAACTGAAATTAATCATGCAGCTGGGAGAGTCCTT
AAAAAGACTACCCCAATGAAACCATGTTACGGTTCATATGCAAAACCCACAAATGAAAATGGAGGGCAAGGCATTCTTGTAAAGCAA
CAAAATGGAAAGCTAGAAAGTCAAGTGGAAATGCAATTTTTCTCAACTACTGAGGCGACCGCAGGCAATGGTGATAACTTGACTCCT
AAAGTGGTATTGTACAGTGAAGATGTAGATATAGAAACCCCAGACACTCATATTTCTTACATGCCCACTATTAAGGAAGGTAACTCA
CGAGAACTAATGGGCCAACAATCTATGCCCAACAGGCCTAATTACATTGCTTTTAGGGACAATTTTATTGGTCTAATGTATTACAAC
AGCACGGGTAATATGGGTGTTCTGGCGGGCCAAGCATCGCAGTTGAATGCTGTTGTAGATTTGCAAGACAGAAACACAGAGCTTTCA
TACCAGCTTTTGCTTGATTCCATTGGTGATAGAACCAGGTACTTTTCTATGTGGAATCAGGCTGTTGACAGCTATGATCCAGATGTT
AGAATTATTGAAAATCATGGAACTGAAGATGAACTTCCAAATTACTGCTTTCCACTGGGAGGTGTGATTAATACAGAGACTCTTACC
AAGGTAAAACCTAAAACAGGTCAGGAAAATGGATGGGAAAAAGATGCTACAGAATTTTCAGATAAAAATGAAATAAGAGTTGGAAAT
AATTTTGCCATGGAAATCAATCTAAATGCCAACCTGTGGAGAAATTTCCTGTACTCCAACATAGCGCTGTATTTGCCCGACAAGCTA
AAGTACAGTCCTTCCAACGTAAAAATTTCTGATAACCCAAACACCTACGACTACATGAACAAGCGAGTGGTGGCTCCCGGGTTAGTG
GACTGCTACATTAACCTTGGAGCACGCTGGTCCCTTGACTATATGGACAACGTCAACCCATTTAACCACCACCGCAATGCTGGCCTG
CGCTACCGCTCAATGTTGCTGGGCAATGGTCGCTATGTGCCCTTCCACATCCAGGTGCCTCAGAAGTTCTTTGCCATTAAAAACCTC
CTTCTCCTGCCGGGCTCATACACCTACGAGTGGAACTTCAGGAAGGATGTTAACATGGTTCTGCAGAGCTCCCTAGGAAATGACCTA
AGGGTTGACGGAGCCAGCATTAAGTTTGATAGCATTTGCCTTTACGCCACCTTCTTCCCCATGGCCCACAACACCGCCTCCACGCTT
GAGGCCATGCTTAGAAACGACACCAACGACCAGTCCTTTAACGACTATCTCTCCGCCGCCAACATGCTCTACCCTATACCCGCCAAC
GCTACCAACGTGCCCATATCCATCCCCTCCCGCAACTGGGCGGCTTTCCGCGGCTGGGCCTTCACGCGCCTTAAGACTAAGGAAACC
CCATCACTGGGCTCGGGCTACGACCCTTATTACACCTACTCTGGCTCTATACCCTACCTAGATGGAACCTTTTACCTCAACCACACC
TTTAAGAAGGTGGCCATTACCTTTGACTCTTCTGTCAGCTGGCCTGGCAATGACCGCCTGCTTACCCCCAACGAGTTTGAAATTAAG
CGCTCAGTTGACGGGGAGGGTTACAACGTTGCCCAGTGTAACATGACCAAAGACTGGTTCCTGGTACAAATGCTAGCTAACTACAAC
ATTGGCTACCAGGGCTTCTATATCCCAGAGAGCTACAAGGACCGCATGTACTCCTTCTTTAGAAACTTCCAGCCCATGAGCCGTCAG
GTGGTGGATGATACTAAATACAAGGACTACCAACAGGTGGGCATCCTACACCAACACAACAACTCTGGATTTGTTGGCTACCTTGCC
CCCACCATGCGCGAAGGACAGGCCTACCCTGCTAACTTCCCCTATCCGCTTATAGGCAAGACCGCAGTTGACAGCATTACCCAGAAA
AAGTTTCTTTGCGATCGCACCCTTTGGCGCATCCCATTCTCCAGTAACTTTATGTCCATGGGCGCACTCACAGACCTGGGCCAAAAC
CTTCTCTACGCCAACTCCGCCCACGCGCTAGACATGACTTTTGAGGTGGATCCCATGGACGAGCCCACCCTTCTTTATGTTTTGTTT
GAAGTCTTTGACGTGGTCCGTGTGCACCGGCCGCACCGCGGCGTCATCGAAACCGTGTACCTGCGCACGCCCTTCTCGGCCGGCAAC
GCCACAACATAA
Ad5-HVR2-DogTag Hexon sequence
Amino acid (SEQ ID NO: 9):
MATPSMMPQWSYMHISGQDASEYLSPGLVQFARATETYFSLNNKFRNPTVAPTHDVTTDRSQRLTLRFIPVDREDTAYSYKARFTLA
VGDNRVLDMASTYFDIRGVLDRGPTFKPYSGTAYNALAPKGAPNPCEWDEAATALEINLEEEDDDNEDEVDEQAEQQKTHVFGQAPY
SGINITKEGIQIGVGSGGSGDIPATYEFTDGKHYITNEPIPPKGSGGSGPKYADKTFQPEPQIGESQWYETEINHAAGRVLKKTTPM
KPCYGSYAKPTNENGGQGILVKQQNGKLESQVEMQFFSTTEATAGNGDNLTPKVVLYSEDVDIETPDTHISYMPTIKEGNSRELMGQ
QSMPNRPNYIAFRDNFIGLMYYNSTGNMGVLAGQASQLNAVVDLQDRNTELSYQLLLDSIGDRTRYFSMWNQAVDSYDPDVRIIENH
GTEDELPNYCFPLGGVINTETLTKVKPKTGQENGWEKDATEFSDKNEIRVGNNFAMEINLNANLWRNFLYSNIALYLPDKLKYSPSN
VKISDNPNTYDYMNKRVVAPGLVDCYINLGARWSLDYMDNVNPFNHHRNAGLRYRSMLLGNGRYVPFHIQVPQKFFAIKNLLLLPGS
YTYEWNFRKDVNMVLQSSLGNDLRVDGASIKFDSICLYATFFPMAHNTASTLEAMLRNDTNDQSFNDYLSAANMLYPIPANATNVPI
SIPSRNWAAFRGWAFTRLKTKETPSLGSGYDPYYTYSGSIPYLDGTFYLNHTFKKVAITFDSSVSWPGNDRLLTPNEFEIKRSVDGE
GYNVAQCNMTKDWFLVQMLANYNIGYQGFYIPESYKDRMYSFFRNFQPMSRQVVDDTKYKDYQQVGILHQHNNSGFVGYLAPTMREG
QAYPANFPYPLIGKTAVDSITQKKFLCDRTLWRIPFSSNFMSMGALTDLGQNLLYANSAHALDMTFEVDPMDEPTLLYVLFEVFDVV
RVHRPHRGVIETVYLRTPFSAGNATT
DNA (incl. STOP) (SEQ ID NO: 10):
ATGGCTACCCCTTCGATGATGCCGCAGTGGTCTTACATGCACATCTCGGGCCAGGACGCCTCGGAGTACCTGAGCCCCGGGCTGGTG
CAGTTTGCCCGCGCCACCGAGACGTACTTCAGCCTGAATAACAAGTTTAGAAACCCCACGGTGGCGCCTACGCACGACGTGACCACA
GACCGGTCCCAGCGTTTGACGCTGCGGTTCATCCCTGTGGACCGTGAGGATACTGCGTACTCGTACAAGGCGCGGTTCACCCTAGCT
GTGGGTGATAACCGTGTGCTGGACATGGCTTCCACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAATGGGATGAAGCTGCTACTGCTCTTGAAATAAAC
CTAGAAGAAGAGGACGATGACAACGAAGACGAAGTAGACGAGCAAGCTGAGCAGCAAAAAACTCACGTATTTGGGCAGGCGCCTTAT
TCTGGTATAAATATTACAAAGGAGGGTATTCAAATAGGTGTCGGCAGCGGAGGATCCGGCgatattccggctacatatgaatttacc
gatggtaaacattatatcaccaatgaaccgataccgccgaaaGGCTCTGGCGGAAGCGGCCCTAAATATGCCGATAAAACATTTCAA
CCTGAACCTCAAATAGGAGAATCTCAGTGGTACGAAACTGAAATTAATCATGCAGCTGGGAGAGTCCTTAAAAAGACTACCCCAATG
AAACCATGTTACGGTTCATATGCAAAACCCACAAATGAAAATGGAGGGCAAGGCATTCTTGTAAAGCAACAAAATGGAAAGCTAGAA
AGTCAAGTGGAAATGCAATTTTTCTCAACTACTGAGGCGACCGCAGGCAATGGTGATAACTTGACTCCTAAAGTGGTATTGTACAGT
GAAGATGTAGATATAGAAACCCCAGACACTCATATTTCTTACATGCCCACTATTAAGGAAGGTAACTCACGAGAACTAATGGGCCAA
CAATCTATGCCCAACAGGCCTAATTACATTGCTTTTAGGGACAATTTTATTGGTCTAATGTATTACAACAGCACGGGTAATATGGGT
GTTCTGGCGGGCCAAGCATCGCAGTTGAATGCTGTTGTAGATTTGCAAGACAGAAACACAGAGCTTTCATACCAGCTTTTGCTTGAT
TCCATTGGTGATAGAACCAGGTACTTTTCTATGTGGAATCAGGCTGTTGACAGCTATGATCCAGATGTTAGAATTATTGAAAATCAT
GGAACTGAAGATGAACTTCCAAATTACTGCTTTCCACTGGGAGGTGTGATTAATACAGAGACTCTTACCAAGGTAAAACCTAAAACA
GGTCAGGAAAATGGATGGGAAAAAGATGCTACAGAATTTTCAGATAAAAATGAAATAAGAGTTGGAAATAATTTTGCCATGGAAATC
AATCTAAATGCCAACCTGTGGAGAAATTTCCTGTACTCCAACATAGCGCTGTATTTGCCCGACAAGCTAAAGTACAGTCCTTCCAAC
GTAAAAATTTCTGATAACCCAAACACCTACGACTACATGAACAAGCGAGTGGTGGCTCCCGGGTTAGTGGACTGCTACATTAACCTT
GGAGCACGCTGGTCCCTTGACTATATGGACAACGTCAACCCATTTAACCACCACCGCAATGCTGGCCTGCGCTACCGCTCAATGTTG
CTGGGCAATGGTCGCTATGTGCCCTTCCACATCCAGGTGCCTCAGAAGTTCTTTGCCATTAAAAACCTCCTTCTCCTGCCGGGCTCA
TACACCTACGAGTGGAACTTCAGGAAGGATGTTAACATGGTTCTGCAGAGCTCCCTAGGAAATGACCTAAGGGTTGACGGAGCCAGC
ATTAAGTTTGATAGCATTTGCCTTTACGCCACCTTCTTCCCCATGGCCCACAACACCGCCTCCACGCTTGAGGCCATGCTTAGAAAC
GACACCAACGACCAGTCCTTTAACGACTATCTCTCCGCCGCCAACATGCTCTACCCTATACCCGCCAACGCTACCAACGTGCCCATA
TCCATCCCCTCCCGCAACTGGGCGGCTTTCCGCGGCTGGGCCTTCACGCGCCTTAAGACTAAGGAAACCCCATCACTGGGCTCGGGC
TACGACCCTTATTACACCTACTCTGGCTCTATACCCTACCTAGATGGAACCTTTTACCTCAACCACACCTTTAAGAAGGTGGCCATT
ACCTTTGACTCTTCTGTCAGCTGGCCTGGCAATGACCGCCTGCTTACCCCCAACGAGTTTGAAATTAAGCGCTCAGTTGACGGGGAG
GGTTACAACGTTGCCCAGTGTAACATGACCAAAGACTGGTTCCTGGTACAAATGCTAGCTAACTACAACATTGGCTACCAGGGCTTC
TATATCCCAGAGAGCTACAAGGACCGCATGTACTCCTTCTTTAGAAACTTCCAGCCCATGAGCCGTCAGGTGGTGGATGATACTAAA
TACAAGGACTACCAACAGGTGGGCATCCTACACCAACACAACAACTCTGGATTTGTTGGCTACCTTGCCCCCACCATGCGCGAAGGA
CAGGCCTACCCTGCTAACTTCCCCTATCCGCTTATAGGCAAGACCGCAGTTGACAGCATTACCCAGAAAAAGTTTCTTTGCGATCGC
ACCCTTTGGCGCATCCCATTCTCCAGTAACTTTATGTCCATGGGCGCACTCACAGACCTGGGCCAAAACCTTCTCTACGCCAACTCC
GCCCACGCGCTAGACATGACTTTTGAGGTGGATCCCATGGACGAGCCCACCCTTCTTTATGTTTTGTTTGAAGTCTTTGACGTGGTC
CGTGTGCACCGGCCGCACCGCGGCGTCATCGAAACCGTGTACCTGCGCACGCCCTTCTCGGCCGGCAACGCCACAACATAA
Ad5-HVR5-DogTag Hexon sequence
Amino acid (SEQ ID NO: 11):
MATPSMMPQWSYMHISGQDASEYLSPGLVQFARATETYFSLNNKFRNPTVAPTHDVTTDRSQRLTLRFIPVDREDTAYSYKARFTLA
VGDNRVLDMASTYFDIRGVLDRGPTFKPYSGTAYNALAPKGAPNPCEWDEAATALEINLEEEDDDNEDEVDEQAEQQKTHVFGQAPY
SGINITKEGIQIGVEGQTPKYADKTFQPEPQIGESQWYETEINHAAGRVLKKTTPMKPCYGSYAKPTNENGGQGILVKQQNGKLESQ
VEMQFFSGSGGSGDIPATYEFTDGKHYITNEPIPPKGSGGSGPKVVLYSEDVDIETPDTHISYMPTIKEGNSRELMGQQSMPNRPNY
IAFRDNFIGLMYYNSTGNMGVLAGQASQLNAVVDLQDRNTELSYQLLLDSIGDRTRYFSMWNQAVDSYDPDVRIIENHGTEDELPNY
CFPLGGVINTETLTKVKPKTGQENGWEKDATEFSDKNEIRVGNNFAMEINLNANLWRNFLYSNIALYLPDKLKYSPSNVKISDNPNT
YDYMNKRVVAPGLVDCYINLGARWSLDYMDNVNPFNHHRNAGLRYRSMLLGNGRYVPFHIQVPQKFFAIKNLLLLPGSYTYEWNFRK
DVNMVLQSSLGNDLRVDGASIKFDSICLYATFFPMAHNTASTLEAMLRNDTNDQSFNDYLSAANMLYPIPANATNVPISIPSRNWAA
FRGWAFTRLKTKETPSLGSGYDPYYTYSGSIPYLDGTFYLNHTFKKVAITFDSSVSWPGNDRLLTPNEFEIKRSVDGEGYNVAQCNM
TKDWFLVQMLANYNIGYQGFYIPESYKDRMYSFFRNFQPMSRQVVDDTKYKDYQQVGILHQHNNSGFVGYLAPTMREGQAYPANFPY
PLIGKTAVDSITQKKFLCDRTLWRIPFSSNFMSMGALTDLGQNLLYANSAHALDMTFEVDPMDEPTLLYVLFEVFDVVRVHRPHRGV
IETVYLRTPFSAGNATT
DNA (incl. STOP) (SEQ ID NO: 12):
ATGGCTACCCCTTCGATGATGCCGCAGTGGTCTTACATGCACATCTCGGGCCAGGACGCCTCGGAGTACCTGAGCCCCGGGCTGGTG
CAGTTTGCCCGCGCCACCGAGACGTACTTCAGCCTGAATAACAAGTTTAGAAACCCCACGGTGGCGCCTACGCACGACGTGACCACA
GACCGGTCCCAGCGTTTGACGCTGCGGTTCATCCCTGTGGACCGTGAGGATACTGCGTACTCGTACAAGGCGCGGTTCACCCTAGCT
GTGGGTGATAACCGTGTGCTGGACATGGCTTCCACGTACTTTGACATCCGCGGCGTGCTGGACAGGGGCCCTACTTTTAAGCCCTAC
TCTGGCACTGCCTACAACGCCCTGGCTCCCAAGGGTGCCCCAAATCCTTGCGAATGGGATGAAGCTGCTACTGCTCTTGAAATAAAC
CTAGAAGAAGAGGACGATGACAACGAAGACGAAGTAGACGAGCAAGCTGAGCAGCAAAAAACTCACGTATTTGGGCAGGCGCCTTAT
TCTGGTATAAATATTACAAAGGAGGGTATTCAAATAGGTGTCGAAGGTCAAACACCTAAATATGCCGATAAAACATTTCAACCTGAA
CCTCAAATAGGAGAATCTCAGTGGTACGAAACTGAAATTAATCATGCAGCTGGGAGAGTCCTTAAAAAGACTACCCCAATGAAACCA
TGTTACGGTTCATATGCAAAACCCACAAATGAAAATGGAGGGCAAGGCATTCTTGTAAAGCAACAAAATGGAAAGCTAGAAAGTCAA
GTGGAAATGCAATTTTTCTCAGGCAGCGGAGGATCCGGCgatattccggctacatatgaatttaccgatggtaaacattatatcacc
aatgaaccgataccgccgaaaGGCTCTGGCGGAAGCGGCCCTAAAGTGGTATTGTACAGTGAAGATGTAGATATAGAAACCCCAGAC
ACTCATATTTCTTACATGCCCACTATTAAGGAAGGTAACTCACGAGAACTAATGGGCCAACAATCTATGCCCAACAGGCCTAATTAC
ATTGCTTTTAGGGACAATTTTATTGGTCTAATGTATTACAACAGCACGGGTAATATGGGTGTTCTGGCGGGCCAAGCATCGCAGTTG
AATGCTGTTGTAGATTTGCAAGACAGAAACACAGAGCTTTCATACCAGCTTTTGCTTGATTCCATTGGTGATAGAACCAGGTACTTT
TCTATGTGGAATCAGGCTGTTGACAGCTATGATCCAGATGTTAGAATTATTGAAAATCATGGAACTGAAGATGAACTTCCAAATTAC
TGCTTTCCACTGGGAGGTGTGATTAATACAGAGACTCTTACCAAGGTAAAACCTAAAACAGGTCAGGAAAATGGATGGGAAAAAGAT
GCTACAGAATTTTCAGATAAAAATGAAATAAGAGTTGGAAATAATTTTGCCATGGAAATCAATCTAAATGCCAACCTGTGGAGAAAT
TTCCTGTACTCCAACATAGCGCTGTATTTGCCCGACAAGCTAAAGTACAGTCCTTCCAACGTAAAAATTTCTGATAACCCAAACACC
TACGACTACATGAACAAGCGAGTGGTGGCTCCCGGGTTAGTGGACTGCTACATTAACCTTGGAGCACGCTGGTCCCTTGACTATATG
GACAACGTCAACCCATTTAACCACCACCGCAATGCTGGCCTGCGCTACCGCTCAATGTTGCTGGGCAATGGTCGCTATGTGCCCTTC
CACATCCAGGTGCCTCAGAAGTTCTTTGCCATTAAAAACCTCCTTCTCCTGCCGGGCTCATACACCTACGAGTGGAACTTCAGGAAG
GATGTTAACATGGTTCTGCAGAGCTCCCTAGGAAATGACCTAAGGGTTGACGGAGCCAGCATTAAGTTTGATAGCATTTGCCTTTAC
GCCACCTTCTTCCCCATGGCCCACAACACCGCCTCCACGCTTGAGGCCATGCTTAGAAACGACACCAACGACCAGTCCTTTAACGAC
TATCTCTCCGCCGCCAACATGCTCTACCCTATACCCGCCAACGCTACCAACGTGCCCATATCCATCCCCTCCCGCAACTGGGCGGCT
TTCCGCGGCTGGGCCTTCACGCGCCTTAAGACTAAGGAAACCCCATCACTGGGCTCGGGCTACGACCCTTATTACACCTACTCTGGC
TCTATACCCTACCTAGATGGAACCTTTTACCTCAACCACACCTTTAAGAAGGTGGCCATTACCTTTGACTCTTCTGTCAGCTGGCCT
GGCAATGACCGCCTGCTTACCCCCAACGAGTTTGAAATTAAGCGCTCAGTTGACGGGGAGGGTTACAACGTTGCCCAGTGTAACATG
ACCAAAGACTGGTTCCTGGTACAAATGCTAGCTAACTACAACATTGGCTACCAGGGCTTCTATATCCCAGAGAGCTACAAGGACCGC
ATGTACTCCTTCTTTAGAAACTTCCAGCCCATGAGCCGTCAGGTGGTGGATGATACTAAATACAAGGACTACCAACAGGTGGGCATC
CTACACCAACACAACAACTCTGGATTTGTTGGCTACCTTGCCCCCACCATGCGCGAAGGACAGGCCTACCCTGCTAACTTCCCCTAT
CCGCTTATAGGCAAGACCGCAGTTGACAGCATTACCCAGAAAAAGTTTCTTTGCGATCGCACCCTTTGGCGCATCCCATTCTCCAGT
AACTTTATGTCCATGGGCGCACTCACAGACCTGGGCCAAAACCTTCTCTACGCCAACTCCGCCCACGCGCTAGACATGACTTTTGAG
GTGGATCCCATGGACGAGCCCACCCTTCTTTATGTTTTGTTTGAAGTCTTTGACGTGGTCCGTGTGCACCGGCCGCACCGCGGCGTC
ATCGAAACCGTGTACCTGCGCACGCCCTTCTCGGCCGGCAACGCCACAACATAA
Ad5-pIX-SpyCatcher sequences
pIX-(EAAAK3)-SpyCatcher sequence:
Amino acid (SEQ ID NO: 13):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKGSDSATHIKFSKRDED
GKELAGATMELRDSSGKTISTWISDGQVKDFYLYPGKYTFVETAAPDGYEVATAITFTVNEQGQVTVNGKATKGDAHI
DNA (incl. STOP) (SEQ ID NO: 14):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAggctccGAAGCC
GCCGCTAAAGAAGCTGCTGCCAAAGAGGCCGCTGCAAAGggatccGACAGCGCCACACACATCAAGTTCAGCAAGAGAGATGAGGAC
GGCAAAGAGCTGGCTGGCGCAACAATGGAACTGAGAGATAGCAGCGGCAAGACCATCAGCACCTGGATCTCCGACGGCCAAGTGAAG
GACTTCTATCTGTACCCCGGCAAGTACACCTTCGTGGAAACCGCCGCTCCTGACGGATATGAAGTGGCCACCGCCATCACCTTCACC
GTGAATGAGCAGGGACAAGTGACCGTGAACGGCAAGGCCACAAAAGGCGACGCCCACATTTAA
Ligands for capsid decoration described in this patent:
Recombinant Proteins:
DogCatcher
Amino acid (SEQ ID NO: 15):
MGSSHHHHHHSSGLVPRGSHMKLGDIEFIKVNKNDKKPLRGAVFSLQKQHPDYPDIYGAIDQNGTYQNVRTGEDGKLTFKNLSDGKY
RLFENSEPAGYKPVQNKPIVAFQIVNGEVRDVTSIVPQ
DNA (incl. STOP) (SEQ ID NO: 16):
ATGGGCAGCAGCCATCATCATCATCATCACAGCAGCGGCCTGGTGCCGCGCGGCAGCCATATGAAACTGGGCGATATTGAATTTATT
AAAGTGAACAAAAACGATAAAAAGCCGCTGCGTGGTGCCGTGTTTAGCCTGCAGAAACAGCATCCCGACTATCCCGATATCTATGGC
GCGATTGATCAGAATGGGACCTATCAAAATGTGCGTACCGGCGAAGATGGTAAACTGACCTTTAAGAATCTGAGCGATGGCAAATAT
CGCCTGTTTGAAAATAGCGAACCCGCTGGCTATAAACCGGTGCAGAATAAGCCGATTGTGGCGTTTCAGATTGTGAATGGCGAAGTG
CGTGATGTGACCAGCATTGTGCCGCAGTAA
DogCatcher-NANP9
Amino acid (SEQ ID NO: 17):
MAHHHHHHVGTGKLGDIEFIKVNKNDKKPLRGAVFSLQKQHPDYPDIYGAIDQNGTYQNVRTGEDGKLTFKNLSDGKYRLFENSEPA
GYKPVQNKPIVAFQIVNGEVRDVTSIVPQGSGGSGGSNANPNANPNANPNANPNANPNANPNANPNANPNANP
DNA (incl. STOP) (SEQ ID NO: 18):
ATGGCACATCACCACCACCATCACGTGGGTACCGGTAAACTGGGCGATATTGAATTTATTAAAGTGAACAAAAACGATAAAAAGCCG
CTGCGTGGTGCCGTGTTTAGCCTGCAGAAACAGCATCCCGACTATCCCGATATCTATGGCGCGATTGATCAGAATGGGACCTATCAA
AATGTGCGTACCGGCGAAGATGGTAAACTGACCTTTAAGAATCTGAGCGATGGCAAATATCGCCTGTTTGAAAATAGCGAACCCGCT
GGCTATAAACCGGTGCAGAATAAGCCGATTGTGGCGTTTCAGATTGTGAATGGCGAAGTGCGTGATGTGACCAGCATTGTGCCGCAG
GGCTCTGGCGGAAGCGGCggatccAATGCGAACCCTAATGCGAATCCCAATGCAAATCCCAATGCGAACCCTAACGCAAATCCGAAC
GCAAACCCTAACGCGAACCCTAATGCTAATCCTAACGCCAATCCTtaa
DogCatcher-NANP18
Amino acid (SEQ ID NO: 19):
MAHHHHHHVGTGKLGDIEFIKVNKNDKKPLRGAVFSLQKQHPDYPDIYGAIDQNGTYQNVRTGEDGKLTFKNLSDGKYRLFENSEPA
GYKPVQNKPIVAFQIVNGEVRDVTSIVPQGSGGSGGSNANPNANPNANPNANPNANPNANPNANPNANPNANPNANPNANPNANPNA
NPNANPNANPNANPNANPNANP
DNA (incl. STOP) (SEQ ID NO: 20):
ATGGCACATCACCACCACCATCACGTGGGTACCGGTAAACTGGGCGATATTGAATTTATTAAAGTGAACAAAAACGATAAAAAGCCG
CTGCGTGGTGCCGTGTTTAGCCTGCAGAAACAGCATCCCGACTATCCCGATATCTATGGCGCGATTGATCAGAATGGGACCTATCAA
AATGTGCGTACCGGCGAAGATGGTAAACTGACCTTTAAGAATCTGAGCGATGGCAAATATCGCCTGTTTGAAAATAGCGAACCCGCT
GGCTATAAACCGGTGCAGAATAAGCCGATTGTGGCGTTTCAGATTGTGAATGGCGAAGTGCGTGATGTGACCAGCATTGTGCCGCAG
GGCTCTGGCGGAAGCGGCggatccAATGCTAACCCTAACGCTAACCCCAACGCCAATCCGAATGCGAATCCTAACGCCAATCCAAAT
GCCAATCCGAACGCGAACCCAAACGCTAATCCAAACGCGAATCCAAATGCGAACCCTAATGCGAATCCCAATGCAAATCCCAATGCG
AACCCTAACGCAAATCCGAACGCAAACCCTAACGCGAACCCTAATGCTAATCCTAACGCCAATCCTtaa
DogCatcher-NANP Domain
Amino acid (SEQ ID NO: 21):
MAHHHHHHVGTGKLGDIEFIKVNKNDKKPLRGAVFSLQKQHPDYPDIYGAIDQNGTYQNVRTGEDGKLTFKNLSDGKYRLFENSEPA
GYKPVQNKPIVAFQIVNGEVRDVTSIVPQGSGGSGGSNANPNVDPNANPNVDPNANPNVDPNANPNANPNANPNANPNANPNANPNA
NPNANPNANPNANPNANPNANPNANPNANPNANPNANPNANPNVDPNANPNANPNANPNANPNANPNANPNANPNANPNANPNANPN
ANPNANPNANPNANPNANPNANPNANPNANP
DNA (incl. STOP) (SEQ ID NO: 22):
ATGGCACATCACCACCACCATCACGTGGGTACCGGTAAACTGGGCGATATTGAATTTATTAAAGTGAACAAAAACGATAAAAAGCCG
CTGCGTGGTGCCGTGTTTAGCCTGCAGAAACAGCATCCCGACTATCCCGATATCTATGGCGCGATTGATCAGAATGGGACCTATCAA
AATGTGCGTACCGGCGAAGATGGTAAACTGACCTTTAAGAATCTGAGCGATGGCAAATATCGCCTGTTTGAAAATAGCGAACCCGCT
GGCTATAAACCGGTGCAGAATAAGCCGATTGTGGCGTTTCAGATTGTGAATGGCGAAGTGCGTGATGTGACCAGCATTGTGCCGCAG
GGCTCTGGCGGAAGCGGCGGATCCAATGCAAATCCGAATGTTGATCCGAACGCGAACCCGAACGTGGACCCTAACGCCAATCCTAAT
GTGGACCCAAATGCGAATCCAAATGCTAACCCAAACGCAAACCCGAATGCGAACCCCAATGCCAATCCGAACGCTAATCCCAATGCT
AATCCTAATGCAAATCCAAACGCGAATCCGAACGCCAATCCTAACGCAAACCCGAACGCAAATCCAAATGCAAACCCAAATGCTAAT
CCTAATGCGAACCCGAATGCTAACCCGAATGCAAACCCTAACGTTGACCCTAATGCTAACCCTAACGCTAACCCCAACGCCAATCCG
AATGCGAATCCTAACGCCAATCCAAATGCCAATCCGAACGCGAACCCAAACGCTAATCCAAACGCGAATCCAAATGCGAACCCTAAT
GCGAATCCCAATGCAAATCCCAATGCGAACCCTAACGCAAATCCGAACGCAAACCCTAACGCGAACCCTAATGCTAATCCTAACGCC
AATCCTTAA
SpyTag-MBP
Amino acid (SEQ ID NO: 23):
MGSSHHHHHHSSGLVPRGSHMGAHIVMVDAYKPTKGSGESGKIEEGKLVIWINGDKGYNGLAEVGKKFEKDTGIKVTVEHPDKLEEK
FPQVAATGDGPDIIFWAHDRFGGYAQSGLLAEITPDKAFQDKLYPFTWDAVRYNGKLIAYPIAVEALSLIYNKDLLPNPPKTWEEIP
ALDKELKAKGKSALMFNLQEPYFTWPLIAADGGYAFKYENGKYDIKDVGVDNAGAKAGLTFLVDLIKNKHMNADTDYSIAEAAFNKG
ETAMTINGPWAWSNIDTSKVNYGVTVLPTFKGQPSKPFVGVLSAGINAASPNKELAKEFLENYLLTDEGLEAVNKDKPLGAVALKSY
EEELAKDPRIAATMENAQKGEIMPNIPQMSAFWYAVRTAVINAASGRQTVDEALKDAQTNSSS
DNA (incl. STOP) (SEQ ID NO: 24):
ATGGGCAGCAGCCATCATCATCATCATCACAGCAGCGGCCTGGTGCCGCGCGGCAGCCATATGGGAGCCCACATCGTGATGGTGGAC
GCCTACAAGCCGACGAAGggtagtggtgaaagtggtAAAATCGAAGAAGGTAAACTGGTAATCTGGATTAACGGCGATAAAGGCTAT
AACGGTCTCGCTGAAGTCGGTAAGAAATTCGAGAAAGATACCGGAATTAAAGTCACCGTTGAGCATCCGGATAAACTGGAAGAGAAA
TTCCCACAGGTTGCGGCAACTGGCGATGGCCCTGACATTATCTTCTGGGCACACGACCGCTTTGGTGGCTACGCTCAATCTGGCCTG
TTGGCTGAAATCACCCCGGACAAAGCGTTCCAGGACAAGCTGTATCCGTTTACCTGGGATGCCGTACGTTACAACGGCAAGCTGATT
GCTTACCCGATCGCTGTTGAAGCGTTATCGCTGATTTATAACAAAGATCTGCTGCCGAACCCGCCAAAAACCTGGGAAGAGATCCCG
GCGCTGGATAAAGAACTGAAAGCGAAAGGTAAGAGCGCGCTGATGTTCAACCTGCAAGAACCGTACTTCACCTGGCCGCTGATTGCT
GCTGACGGGGGTTATGCGTTCAAGTATGAAAACGGCAAGTACGACATTAAAGACGTGGGCGTGGATAACGCTGGCGCGAAAGCGGGT
CTGACCTTCCTGGTTGACCTGATTAAAAACAAACACATGAATGCAGACACCGATTACTCCATCGCAGAAGCTGCCTTTAATAAAGGC
GAAACAGCGATGACCATCAACGGCCCGTGGGCATGGTCCAACATCGACACCAGCAAAGTGAATTATGGTGTAACGGTACTGCCGACC
TTCAAGGGTCAACCATCCAAACCGTTCGTTGGCGTGCTGAGCGCAGGTATTAACGCCGCCAGTCCGAACAAAGAGCTGGCAAAAGAG
TTCCTCGAAAACTATCTGCTGACTGATGAAGGTCTGGAAGCGGTTAATAAAGACAAACCGCTGGGTGCCGTAGCGCTGAAGTCTTAC
GAGGAAGAGTTGGCGAAAGATCCACGTATTGCCGCCACTATGGAAAACGCCCAGAAAGGTGAAATCATGCCGAACATCCCGCAGATG
TCCGCTTTCTGGTATGCCGTGCGTACTGCGGTGATCAACGCCGCCAGCGGTCGTCAGACTGTCGATGAAGCCCTGAAAGACGCGCAG
ACTAATTCGAGCTCGTAA
SnoopLigase
Amino acid (SEQ ID NO: 25):
MGSWSHHHHHHSSGGSGVNKNDKKPLRGAVFSLQKQHPDYPDIYGAIDQNGTYQNVRTGEDGKLTFKNLSDGKYRLFENSEPPGYKP
VQNKPIVAFQIVNGEVRDVTSIVPPGVPATYEFT
DNA (incl. STOP) (SEQ ID NO: 26):
atgggcagctggagccatcatcatcatcatcacagctctggtggtagtggtgtgaataagaacgataaaaagccgctgcgtggtgcc
gtgtttagcctgcagaaacagcatcccgactatcccgatatctatggcgcgattgatcagaatgggacctatcaaaatgtgcgtacc
ggcgaagatggtaaactgacctttaagaatctgagcgatggcaaatatcgcctgtttgaaaatagcgaacccccgggctataaaccg
gtgcagaataagccgattgtggcgtttcagattgtgaatggcgaagtgcgtgatgtgaccagcattgtgccgccgggtgtgccggct
acatatgaatttacctaa
SnoopTagJr-AffiHER2
Amino acid (SEQ ID NO: 27):
MGSSHHHHHHSSGGKLGSIEFIKVNKGSGESGSGASMTGGQQMGRDPGVDNKFNKEMRNAYWEIALLPNLNNQQKRAFIRSLYDDPS
QSANLLAEAKKLNDAQAPKGLE
DNA (incl. STOP) (SEQ ID NO: 28):
atgggcagcagccatcatcatcatcatcacagcagcggcgggaaactgggctctattgaatttattaaagtgaacaaaggcagtggt
gagtcgggatccggagctagcatgactggtggacagcaaatgggtcgggatccgggcgtggacaacaaattcaacaaagaaatgagg
aacgcttactgggagatagctcttttacccaacttaaacaatcaacagaaaagggctttcataaggtcgttatacgatgacccaagc
caaagcgctaaccttttagcagaagctaaaaagctaaatgatgctcaggcgccgaaaggcctcgagtaa
SpyCatcher
Amino acid (SEQ ID NO: 29):
MSYYHHHHHHDYDIPTTENLYFQGAMVDTLSGLSSEQGQSGDMTIEEDSATHIKFSKRDEDGKELAGATMELRDSSGKTISTWISDG
QVKDFYLYPGKYTFVETAAPDGYEVATAITFTVNEQGQVTVNGKATKGDAHI
DNA (incl. STOP) (SEQ ID NO: 30):
ATGTCGTACTACCATCACCATCACCATCACGATTACGACATCCCAACGACCGAAAACCTGTATTTTCAGGGCGCCATGGTTGATACC
TTATCAGGTTTATCAAGTGAGCAAGGTCAGTCCGGTGATATGACAATTGAAGAAGATAGTGCTACCCATATTAAATTCTCAAAACGT
GATGAGGACGGCAAAGAGTTAGCTGGTGCAACTATGGAGTTGCGTGATTCATCTGGTAAAACTATTAGTACATGGATTTCAGATGGA
CAAGTGAAAGATTTCTACCTGTATCCAGGAAAATATACATTTGTCGAAACCGCAGCACCAGACGGTTATGAGGTAGCAACTGCTATT
ACCTTTACAGTTAATGAGCAAGGTCAGGTTACTGTAAATGGCAAAGCAACTAAAGGTGACGCTCATATTTAA
HCMV Pentamer
HCMV gH-SpyTag
Amino acid (SEQ ID NO: 31):
MRPGLPSYLIILAVCLFSHLLSSRYGAEAVSEPLDKAFHLLLNTYGRPIRFLRENTTQCTYNSSLRNSTVVRENAISFNFFQSYNQY
YVFHMPRCLFAGPLAEQFLNQVDLTETLERYQQRLNTYALVSKDLASYRSFSQQLKAQDSLGEQPTTVPPPIDLSIPHVWMPPQTTP
HGWTESHTTSGLHRPHFNQTCILFDGHDLLFSTVTPCLHQGFYLIDELRYVKITLTEDFFVVTVSIDDDTPMLLIFGHLPRVLFKAP
YQRDNFILRQTEKHELLVLVKKDQLNRHSYLKDPDFLDAALDFNYLDLSALLRNSFHRYAVDVLKSGRCQMLDRRTVEMAFAYALAL
FAAARQEEAGAQVSVPRALDRQAALLQIQEFMITCLSQTPPRTTLLLYPTAVDLAKRALWTPNQITDITSLVRLVYILSKQNQQHLI
PQWALRQIADFALKLHKTHLASFLSAFARQELYLMGSLVHSMLVHTTERREIFIVETGLCSLAELSHFTQLLAHPHHEYLSDLYTPC
SSSGRRDHSLERLTRLFPDATVPATVPAALSILSTMQPSTLETFPDLFCLPLGESFSALTVSEHVSYIVTNQYLIKGISYPVSTTVV
GQSLIITQTDSQTKCELTRNMHTTHSITVALNISLENCAFCQSALLEYDDTQGVINIMYMHDSDDVLFALDPYNEVVVSSPRTHYLM
LLKNGTVLEVTDVVVDATDSRLLGSGGSGAHIVMVDAYKPTKHHHHHH
DNA (incl. STOP) (SEQ ID NO: 32):
ATGCGGCCAGGCCTCCCCTCCTACCTCATCATCCTCGCCGTCTGTCTCTTCAGCCACCTACTTTCGTCACGATATGGCGCAGAAGCC
GTATCCGAACCGCTGGACAAAGCGTTTCACCTACTGCTCAACACCTACGGGAGACCCATCCGCTTCCTGCGTGAAAATACCACCCAG
TGTACCTACAACAGCAGCCTCCGTAACAGCACGGTCGTCAGGGAAAACGCCATCAGTTTCAACTTTTTCCAAAGCTATAATCAATAC
TATGTATTCCATATGCCTCGATGTCTTTTTGCGGGTCCTCTGGCGGAGCAGTTTCTGAACCAGGTAGATCTGACCGAAACCCTGGAA
AGATACCAACAGAGACTTAACACTTACGCGCTGGTATCCAAAGACCTGGCCAGCTACCGATCTTTTTCGCAGCAGCTAAAGGCACAA
GACAGCCTAGGTGAACAGCCCACCACTGTGCCACCGCCCATTGACCTGTCAATACCTCACGTTTGGATGCCACCGCAAACCACTCCA
CACGGCTGGACAGAATCACATACCACCTCAGGACTACACCGACCACACTTTAACCAGACCTGTATCCTCTTTGATGGACACGATCTA
CTATTCAGCACCGTCACACCTTGTTTGCACCAAGGCTTTTACCTCATCGACGAACTACGTTACGTTAAAATAACACTGACCGAGGAC
TTCTTCGTAGTTACGGTGTCCATAGACGACGACACACCCATGCTGCTTATCTTCGGCCATCTTCCACGCGTACTTTTCAAAGCGCCC
TATCAACGCGACAACTTTATACTACGACAAACTGAAAAACACGAGCTCCTGGTGCTAGTTAAGAAAGATCAACTGAACCGTCACTCT
TATCTCAAAGACCCGGACTTTCTTGACGCCGCACTTGACTTCAACTACCTAGACCTCAGCGCACTACTACGTAACAGCTTTCACCGT
TACGCCGTGGATGTACTCAAGAGCGGTCGATGTCAGATGCTGGACCGCCGCACGGTAGAAATGGCCTTCGCCTACGCATTAGCACTG
TTCGCAGCAGCCCGACAAGAAGAGGCCGGCGCCCAAGTCTCCGTCCCACGGGCCCTAGACCGCCAGGCCGCACTCTTACAAATACAA
GAATTTATGATCACATGCCTCTCACAAACACCACCACGCACCACGTTGCTGCTGTATCCCACGGCCGTGGACCTGGCCAAACGAGCC
CTTTGGACACCGAATCAGATCACCGACATCACCAGCCTCGTACGCCTGGTCTACATACTCTCTAAACAGAATCAGCAACATCTCATC
CCCCAATGGGCACTACGACAGATCGCCGACTTTGCCCTAAAACTACACAAAACGCACCTGGCCTCTTTTCTTTCAGCCTTCGCACGC
CAAGAACTCTACCTCATGGGCAGCCTCGTCCACTCCATGCTGGTACATACGACGGAGAGACGCGAAATCTTCATCGTAGAAACGGGC
CTCTGTTCATTGGCCGAGCTATCACACTTTACGCAGTTGTTAGCTCATCCACACCACGAATACCTCAGCGACCTGTACACACCCTGT
TCCAGTAGCGGGCGACGCGATCACTCGCTCGAACGCCTCACGCGTCTCTTCCCCGATGCCACCGTCCCCGCTACCGTTCCCGCCGCC
CTCTCCATCCTATCTACCATGCAACCAAGCACGCTGGAAACCTTCCCCGACCTGTTTTGCTTGCCGCTCGGCGAATCCTTCTCCGCG
CTGACCGTCTCCGAACACGTCAGTTATATCGTAACAAACCAGTACCTGATCAAAGGTATCTCCTACCCTGTCTCCACCACCGTCGTA
GGCCAGAGCCTCATCATCACCCAGACGGACAGTCAAACTAAATGCGAACTGACGCGCAACATGCATACCACACACAGCATCACAGTG
GCGCTCAACATTTCGCTAGAAAACTGCGCCTTTTGCCAAAGCGCCCTGCTAGAATACGACGACACGCAAGGCGTCATCAACATCATG
TACATGCACGACTCGGACGACGTCCTTTTCGCCCTGGATCCCTACAACGAAGTGGTGGTCTCATCTCCGCGAACTCACTACCTCATG
CTTTTGAAAAACGGTACGGTACTAGAAGTAACTGACGTCGTCGTGGACGCCACCGACAGTCGTCTCCTCGGAAGCGGAGGCTCTGGT
GCCCATATCGTGATGGTGgacgCCTACAAGCCTACCAAACATCATCACCATCACCACTAA
HCMV gL
Amino acid (SEQ ID NO: 33):
MCRRPDCGFSFSPGPVILLWCCLLLPIVSSAAVSVAPTAAEKVPAECPELTRRCLLGEVFEGDKYESWLRPLVNVTGRDGPLSQLIR
YRPVTPEAANSVLLDEAFLDTLALLYNNPDQLRALLTLLSSDTAPRWMTVMRGYSECGDGSPAVYTCVDDLCRGYDLTRLSYGRSIF
TEHVLGFELVPPSLFNVVVAIRNEATRTNRAVRLPVSTAAAPEGITLFYGLYNAVKEFCLRHQLDPPLLRHLDKYYAGLPPELKQTR
VNLPAHSRYGPQAVDAR
DNA (incl. STOP) (SEQ ID NO: 34):
ATGTGCCGCCGCCCGGATTGCGGCTTCTCTTTCTCACCTGGACCGGTGATACTGCTGTGGTGTTGCCTTCTGCTGCCCATTGTTTCC
TCAGCCGCCGTCAGCGTCGCTCCTACCGCCGCCGAGAAAGTCCCCGCGGAGTGCCCCGAACTAACGCGCCGATGCTTGTTGGGTGAG
GTGTTTGAGGGTGACAAGTATGAAAGTTGGCTGCGCCCGTTGGTGAATGTTACCGGGCGCGATGGCCCGCTATCGCAACTTATCCGT
TACCGTCCCGTTACGCCGGAGGCCGCCAACTCCGTGCTGTTGGACGAGGCTTTCCTGGACACTCTGGCCCTGCTGTACAACAATCCG
GATCAATTGCGGGCCCTGCTGACGCTGTTGAGCTCGGACACAGCGCCGCGCTGGATGACGGTGATGCGCGGCTACAGCGAGTGCGGC
GATGGCTCGCCGGCCGTGTACACGTGCGTGGACGACCTGTGCCGCGGCTACGACCTCACGCGACTGTCATACGGGCGCAGCATCTTC
ACGGAACACGTGTTAGGCTTCGAGCTGGTGCCACCGTCTCTCTTTAACGTGGTGGTGGCCATACGCAACGAAGCCACGCGTACCAAC
CGCGCCGTGCGTCTGCCCGTGAGCACCGCTGCCGCGCCCGAGGGCATCACGCTCTTTTACGGCCTGTACAACGCAGTGAAGGAATTC
TGCCTGCGTCACCAGCTGGACCCGCCGCTGCTACGCCACCTAGATAAATACTACGCCGGACTGCCGCCCGAGCTGAAGCAGACGCGC
GTCAACCTGCCGGCTCACTCGCGCTATGGCCCTCAAGCAGTGGATGCTCGCTAA
HCMV UL131A
Amino acid (SEQ ID NO: 35):
MRLCRVWLSVCLCAVVLGQCQRETAEKNDYYRVPHYWDACSRALPDQTRYKYVEQLVDLTLNYHYDASHGLDNFDVLKRINVTEVSL
LISDFRRQNRRGGTNKRTTFNAAGSLAPHARSLEFSVRLFAN
DNA (incl. STOP)-contains intron (bold) (SEQ ID NO: 36):
ATGCGGCTGTGTCGGGTGTGGCTGTCTGTTTGTCTGTGCGCCGTGGTGCTGGGTCAGTGCCAGCGGGAAACCGCGGAAAAAAACGAT
TATTACCGAGTACCGCATTACTGGGACGCGTGCTCTCGCGCGCTGCCCGACCAAACCCGTTACAAGTATGTGGAACAGCTCGTGGAC
CTCACGTTGAACTACCACTACGATGCGAGCCACGGCTTGGACAACTTTGACGTGCTCAAGAGGTGAGGGTACGCGCTAAAGATGCAT
GACAACGGGAAGGTAAGGGCGAACGGGTAACGGGTAAGTAACCGCATGGGGTATGAAATGACGTTCGGAACCTGTGCTTGCAGAATC
AACGTGACCGAGGTGTCGTTGCTCATCAGCGACTTTAGACGTCAGAACCGTCGCGGCGGCACCAACAAAAGGACCACGTTCAACGCC
GCCGGTTCGCTGGCGCCACACGCCCGGAGCCTCGAGTTCAGCGTGCGGCTCTTTGCCAACTAG
HCMV UL128
Amino acid (SEQ ID NO: 37):
MSPKDLTPFLTALWLLLGHSRVPRVRAEECCEFINVNHPPERCYDFKMCNRFTVALRCPDGEVCYSPEKTAEIRGIVTTMTHSLTRQ
VVHNKLTSCNYNPLYLEADGRIRCGKVNDKAQYLLGAAGSVPYRWINLEYDKITRIVGLDQYLESVKKHKRLDVCRAKMGYMLQ
DNA (incl. STOP)-contains introns (bold) (SEQ ID NO: 38):
ATGAGTCCCAAAGATCTGACGCCGTTCTTGACGGCGTTGTGGCTGCTATTGGGTCACAGCCGCGTGCCGCGGGTGCGCGCAGAAGAA
TGTTGCGAATTCATAAACGTCAACCACCCGCCGGAACGCTGTTACGATTTCAAAATGTGCAATCGCTTCACCGTCGCGTACGTATTT
TCATGATTGTCTGCGTTCTGTGGTGCGTCTGGATCTGTCTCTCGACGTTTCTGATAGCCATGTTCCATCGACGATCCTCGGGAATGC
CAGAGTAGATTTTCATGAATCCACAGGCTGCGGTGTCCGGACGGCGAAGTCTGCTACAGTCCCGAGAAAACGGCTGAGATTCGCGGG
ATCGTCACCACCATGACCCATTCATTGACACGCCAGGTCGTACACAACAAACTGACGAGCTGCAACTACAATCCGTAAGTCTCTTCC
TGAGGGCCTTACAGCCTATGGGAGAGTAAGACAGAGAGGGACAAAACATCATTAAAAAAAAAAGTCTAATTTCACGTTTTGTACCCC
CCTTCCCCTCCGTGTTGTAGGTTATACCTCGAAGCTGACGGGCGAATACGCTGCGGCAAAGTAAACGACAAGGCGCAGTACCTGCTG
GGCGCCGCTGGCAGCGTTCCCTATcGATGGATCAATCTGGAATACGACAAGATAACCCGGATCGTGGGCCTGGATCAGTACCTGGAG
AGCGTTAAGAAACACAAACGGCTGGATGTGTGCCGCGCTAAAATGGGCTATATGCTGCAGTGA
HCMV UL130
Amino acid (SEQ ID NO: 39):
MLRLLLRHHFHCLLLCAVWATPCLASPWSTLTANQNPSPPWSKLTYSKPHDAATFYCPFLYPSPPRSPLQFSGFQRVSTGPECRNET
LYLLYNREGQTLVERSSTWVKKVIWYLSGRNQTILQRMPRTASKPSDGNVQISVEDAKIFGAHMVPKQTKLLRFVVNDGTRYQMCVM
KLESWAHVFRDYSVSFQVRLTFTEANNQTYTFCTHPNLIVGGGGSGGGGSGGGGSEPEA
DNA (incl. STOP) (SEQ ID NO: 40):
ATGCTGCGGCTTCTGCTTCGTCACCACTTTCACTGCCTGCTTCTGTGCGCGGTTTGGGCAACGCCCTGTCTGGCGTCTCCGTGGTCG
ACGCTAACAGCAAACCAGAATCCGTCCCCGCCATGGTCTAAACTGACGTATTCCAAACCGCATGACGCGGCGACGTTTTACTGTCCT
TTTCTCTATCCCTCGCCCCCACGATCCCCCTTGCAATTCTCGGGGTTCCAGCGGGTATCAACGGGTCCCGAGTGTCGCAACGAGACC
CTGTATCTGCTGTACAACCGGGAAGGCCAGACCTTGGTGGAGAGAAGCTCCACCTGGGTGAAAAAGGTGATCTGGTACCTGAGCGGT
CGGAACCAAACCATCCTCCAACGGATGCCCCGAACGGCTTCGAAACCGAGCGACGGAAACGTGCAGATCAGCGTGGAAGACGCCAAG
ATTTTTGGAGCGCACATGGTGCCCAAGCAGACCAAGCTGCTACGCTTCGTCGTCAACGATGGCACACGTTATCAGATGTGTGTGATG
AAGCTGGAGAGCTGGGCTCACGTCTTCCGGGACTACAGCGTGTCTTTTCAGGTGCGATTGACGTTCACCGAGGCCAATAACCAGACT
TACACCTTCTGCACCCATCCCAATCTCATCGTTGGAGGCGGAGGATCTGGCGGAGGTGGAAGTGGCGGAGGCGGATCTGAGCCCGAG
GCCTAA
Peptides (solid phase synthesized-amino acid sequences only):
SnoopTagJr-hTERT (SEQ ID NO: 41): GKLGSIEFIKVNKGEARPALLTSRLRFIPK
SnoopTagJr-GGS-SHNFEKL (PEP1) (SEQ ID NO: 42): GKLGSIEFIKVNKGGGSSIINFEKL
SnoopTagJr-AAY-SHNFEKL (PEP2) (SEQ ID NO: 43): GKLGSIEFIKVNKGAAYSIINFEKL
Biotin-SnoopTagJr (SEQ ID NO: 44): Biotin-GKLGSIEFIKVNK (N-terminal biotin)
Biotin-SpyTag003 (SEQ ID NO: 45): Biotin-GSRGVPHIVMVDAYKRYK (N-terminal biotin)
Modified adenovirus sequences:
Ad5-pIX-SpyCatcher sequences
Ad5-pIX-SpyCatcher (no linker) pIX sequence
Amino acid (SEQ ID NO: 46):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVDSATHIKFSKRDEDGKELAGATMELRDSSGKTIS
TWISDGQVKDFYLYPGKYTFVETAAPDGYEVATAITFTVNEQGQVTVNGKATKGDAHI
DNA (incl. STOP) (SEQ ID NO: 47):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGACAGCGCCACACAC
ATCAAGTTCAGCAAGAGAGATGAGGACGGCAAAGAGCTGGCTGGCGCAACAATGGAACTGAGAGATAGCAGCGGCAAGACCATCAGC
ACCTGGATCTCCGACGGCCAAGTGAAGGACTTCTATCTGTACCCCGGCAAGTACACCTTCGTGGAAACCGCCGCTCCTGACGGATAT
GAAGTGGCCACCGCCATCACCTTCACCGTGAATGAGCAGGGACAAGTGACCGTGAACGGCAAGGCCACAAAAGGCGACGCCCACATT
TAA
Ad5-pIX-SpyCatcher (EAAAK3-GS linker) pIX sequence
Amino acid (SEQ ID NO: 48):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVEAAAKEAAAKEAAAKGSDSATHIKFSKRDEDGKE
LAGATMELRDSSGKTISTWISDGQVKDFYLYPGKYTFVETAAPDGYEVATAITFTVNEQGQVTVNGKATKGDAHI
DNA (incl. STOP) (SEQ ID NO: 49):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGAAGCCGCCGCTAAA
GAAGCTGCTGCCAAAGAGGCCGCTGCAAAGggatCCGACAGCGCCACACACATCAAGTTCAGCAAGAGAGATGAGGACGGCAAAGAG
CTGGCTGGCGCAACAATGGAACTGAGAGATAGCAGCGGCAAGACCATCAGCACCTGGATCTCCGACGGCCAAGTGAAGGACTTCTAT
CTGTACCCCGGCAAGTACACCTTCGTGGAAACCGCCGCTCCTGACGGATATGAAGTGGCCACCGCCATCACCTTCACCGTGAATGAG
CAGGGACAAGTGACCGTGAACGGCAAGGCCACAAAAGGCGACGCCCACATTTAA
Ad5-pIX-SpyCatcher (GGS-EAAAK3 linker) pIX sequence
Amino acid (SEQ ID NO: 50):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKDSATHIKFSKRDEDGK
ELAGATMELRDSSGKTISTWISDGQVKDFYLYPGKYTFVETAAPDGYEVATAITFTVNEQGQVTVNGKATKGDAHI
DNA (incl. STOP) (SEQ ID NO: 51):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAggCtCCGAAGCC
GCCGCTAAAGAAGCTGCTGCCAAAGAGGCCGCTGCAAAGGACAGCGCCACACACATCAAGTTCAGCAAGAGAGATGAGGACGGCAAA
GAGCTGGCTGGCGCAACAATGGAACTGAGAGATAGCAGCGGCAAGACCATCAGCACCTGGATCTCCGACGGCCAAGTGAAGGACTTC
TATCTGTACCCCGGCAAGTACACCTTCGTGGAAACCGCCGCTCCTGACGGATATGAAGTGGCCACCGCCATCACCTTCACCGTGAAT
GAGCAGGGACAAGTGACCGTGAACGGCAAGGCCACAAAAGGCGACGCCCACATTTAA
Ad5-pIX-SpyCatcher (EAAAK3 linker, no GGS or GS hinges) pIX sequence
Amino acid (SEQ ID NO: 52):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVEAAAKEAAAKEAAAKDSATHIKFSKRDEDGKELA
GATMELRDSSGKTISTWISDGQVKDFYLYPGKYTFVETAAPDGYEVATAITFTVNEQGQVTVNGKATKGDAHI
DNA (incl. STOP) (SEQ ID NO: 53):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGAAGCCGCCGCTAAA
GAAGCTGCTGCCAAAGAGGCCGCTGCAAAGGACAGCGCCACACACATCAAGTTCAGCAAGAGAGATGAGGACGGCAAAGAGCTGGCT
GGCGCAACAATGGAACTGAGAGATAGCAGCGGCAAGACCATCAGCACCTGGATCTCCGACGGCCAAGTGAAGGACTTCTATCTGTAC
CCCGGCAAGTACACCTTCGTGGAAACCGCCGCTCCTGACGGATATGAAGTGGCCACCGCCATCACCTTCACCGTGAATGAGCAGGGA
CAAGTGACCGTGAACGGCAAGGCCACAAAAGGCGACGCCCACATTTAA
Ad5-pIX-SpyCatcher (GGS-EAAAK5-GS linker) pIX sequence
Amino acid (SEQ ID NO: 54):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKEAAAKEAAAKGSDSAT
HIKFSKRDEDGKELAGATMELRDSSGKTISTWISDGQVKDFYLYPGKYTFVETAAPDGYEVATAITFTVNEQGQVTVNGKATKGDAH
I
DNA (incl. STOP) (SEQ ID NO: 55):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAGGCTCCGAAGCC
GCCGCTAAAGAAGCTGCTGCCAAAGAGGCCGCTGCAAAGGAGGCCGCAGCTAAGGAAGCCGCCGCTAAGGGATCCGACAGCGCCACA
CACATCAAGTTCAGCAAGAGAGATGAGGACGGCAAAGAGCTGGCTGGCGCAACAATGGAACTGAGAGATAGCAGCGGCAAGACCATC
AGCACCTGGATCTCCGACGGCCAAGTGAAGGACTTCTATCTGTACCCCGGCAAGTACACCTTCGTGGAAACCGCCGCTCCTGACGGA
TATGAAGTGGCCACCGCCATCACCTTCACCGTGAATGAGCAGGGACAAGTGACCGTGAACGGCAAGGCCACAAAAGGCGACGCCCAC
ATTTAA
Ad5-pIX-SpyCatcher(deltaN1deltaC2) pIX sequence
Amino acid (SEQ ID NO: 56):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKGSDSATHIKFSKRDED
GKELAGATMELRDSSGKTISTWISDGQVKDFYLYPGKYTFVETAAPDGYEVATAITFTVNEQGQVTVNG
DNA (incl. STOP) (SEQ ID NO: 57):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAggctccGAAGCC
GCCGCTAAAGAAGCTGCTGCCAAAGAGGCCGCTGCAAAGggatccGACAGCGCCACCCACATCAAGTTCAGCAAGAGGGACGAGGAC
GGCAAGGAGCTGGCCGGCGCAACAATGGAGCTGAGGGACAGCAGCGGCAAGACCATCAGCACCTGGATCAGCGACGGCCAGGTGAAG
GACTTCTACCTGTACCCCGGCAAGTACACCTTCGTGGAGACCGCCGCCCCCGACGGCTACGAGGTGGCCACCGCCATCACCTTCACC
GTGAACGAGCAGGGCCAGGTGACCGTGAACGGCTAA
Ad5-pIX-SnoopCatcher sequences
Ad5-pIX-SnoopCatcher pIX sequence
Amino acid (SEQ ID NO: 58):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKGSHMKPLRGAVFSLQK
QHPDYPDIYGAIDQNGTYQNVRTGEDGKLTFKNLSDGKYRLFENSEPAGYKPVQNKPIVAFQIVNGEVRDVTSIVPQDIPATYEFTN
GKHYITNEPIPPK
DNA (incl. STOP) (SEQ ID NO: 59):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAggctccGAAGCC
GCCGCTAAAGAAGCTGCTGCCAAAGAGGCCGCTGCAAAGggatccCACATGAAGCCCCTGAGGGGCGCCGTGTTCAGCCTGCAGAAG
CAGCACCCCGACTACCCCGACATCTACGGCGCCATCGACCAGAACGGCACCTACCAGAACGTGAGGACCGGCGAGGACGGCAAGCTG
ACCTTCAAGAACCTGAGCGACGGCAAGTACAGGCTGTTCGAGAACAGCGAGCCCGCCGGCTACAAGCCCGTGCAGAACAAGCCCATC
GTGGCCTTCCAGATCGTGAACGGCGAGGTGAGGGACGTGACCAGCATCGTGCCCCAGGACATCCCCGCCACCTACGAGTTCACCAAC
GGCAAGCACTACATCACCAACGAGCCCATCCCCCCCAAGTAA
Ad5-pIX-DogCatcher sequences
Ad5-pIX-DogCatcher pIX sequence
Amino acid (SEQ ID NO: 60):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKGSKLGDIEFIKVNKND
KKPLRGAVFSLQKQHPDYPDIYGAIDQNGTYQNVRTGEDGKLTFKNLSDGKYRLFENSEPAGYKPVQNKPIVAFQIVNGEVRDVTSI
VPQ
DNA (incl. STOP) (SEQ ID NO: 61):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAggctccGAAGCC
GCCGCTAAAGAAGCTGCTGCCAAAGAGGCCGCTGCAAAGggatccAAACTGGGCGATATTGAATTTATTAAAGTGAACAAAAACGAT
AAAAAGCCGCTGCGTGGTGCCGTGTTTAGCCTGCAGAAACAGCATCCCGACTATCCCGATATCTATGGCGCGATTGATCAGAATGGG
ACCTATCAAAATGTGCGTACCGGCGAAGATGGTAAACTGACCTTTAAGAATCTGAGCGATGGCAAATATCGCCTGTTTGAAAATAGC
GAACCCGCTGGCTATAAACCGGTGCAGAATAAGCCGATTGTGGCGTTTCAGATTGTGAATGGCGAAGTGCGTGATGTGACCAGCATT
GTGCCGCAGTAA
Ad5-pIX-SpyTag sequences
Ad5-pIX-SpyTag pIX sequence
Amino acid (SEQ ID NO: 62):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKGSAHIVMVDAYKPTK
DNA (incl. STOP) (SEQ ID NO: 63):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAGGCTCCGAAGCC
GCCGCTAAAGAAGCTGCTGCCAAAGAGGCCGCTGCAAAGGGATCCGCCCACATCGTTATGGTGGATGCCTACAAGCCCACCAAATAA
Ad5-pIX-SnoopTagJr sequences
Ad5-pIX-SnoopTagJr pIX sequence
Amino acid (SEQ ID NO: 64):
MSTNSFDGSIVSSYLTTRMPPWAGVRQNVMGSSIDGRPVLPANSTTLTYETVSGTPLETAASAAASAAAATARGIVTDFAFLSPLAS
SAASRSSARDDKLTALLAQLDSLTRELNVVSQQLLDLRQQVSALKASSPPNAVGGSEAAAKEAAAKEAAAKGSKLGSIEFIKVNK
DNA (incl. STOP) (SEQ ID NO: 65):
ATGAGCACCAACTCGTTTGATGGAAGCATTGTGAGCTCATATTTGACAACGCGCATGCCCCCATGGGCCGGGGTGCGTCAGAATGTG
ATGGGCTCCAGCATTGATGGTCGCCCCGTCCTGCCCGCAAACTCTACTACCTTGACCTACGAGACCGTGTCTGGAACGCCGTTGGAG
ACTGCAGCCTCCGCCGCCGCTTCAGCCGCTGCAGCCACCGCCCGCGGGATTGTGACTGACTTTGCTTTCCTGAGCCCGCTTGCAAGC
AGTGCAGCTTCCCGTTCATCCGCCCGCGATGACAAGTTGACGGCTCTTTTGGCACAATTGGATTCTTTGACCCGGGAACTTAATGTC
GTTTCTCAGCAGCTGTTGGATCTGCGCCAGCAGGTTTCTGCCCTGAAGGCTTCCTCCCCTCCCAATGCGGTTGGAGGCTCCGAAGCC
GCCGCTAAAGAAGCTGCTGCCAAAGAGGCCGCTGCAAAGGGATCCaaactgggctctattgaatttattaaagtgaacaaaTA
Ligands for capsid decoration described in this application:
Recombinant Proteins:
SARS CoV2 Spike-SnoopTagJr
Amino acid (SEQ ID NO: 66):
MFVFLVLLPLVSSQCVNLTTRTQLPPAYTNSFTRGVYYPDKVFRSSVLHSTQDLFLPFFSNVTWFHAIHVSGTNGTKRFDNPVLPFN
DGVYFASTEKSNIIRGWIFGTTLDSKTQSLLIVNNATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLVDLPIGINITRFQTLLALHRSYLTPGDSSSGWTAG
AAAYYVGYLQPRTFLLKYNENGTITDAVDCALDPLSETKCTLKSFTVEKGIYQTSNFRVQPTESIVRFPNITNLCPFGEVFNATRFA
SVYAWNRKRISNCVADYSVLYNSASFSTFKCYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGFNCYFPLQSYGFQPTNGVGYQPYRVVVLSFELLHAPA
TVCGPKKSTNLVKNKCVNFNFNGLTGTGVLTESNKKFLPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLGDIAARDLICAQKFNGLTVLPPLLTDEMI
AQYTSALLAGTITSGWTFGAGAALQIPFAMQMAYRFNGIGVTQNVLYENQKLIANQFNSAIGKIQDSLSSTASALGKLQDVVNQNAQ
ALNTLVKQLSSNFGAISSVLNDILSRLDPPEAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQKEIDRLNEVAKNLNESLIDLQELGKYEQGSGIVQQQNN
LLRAIEAQQHLLQLTVWGIKQLQARILAGGSGGHTTWMEWDREINNYTSLIHSLIEESQNQQEKNEQELLEGSGGSGKLGSIEFIKV
NKEPEA
DNA (incl. STOP) (SEQ ID NO: 67):
ATGTTCGTGTTTCTGGTGCTGCTGCCTCTGGTGTCCAGCCAGTGTGTGAACCTGACCACCAGAACACAGCTGCCTCCAGCCTACACC
AACAGCTTTACCAGAGGCGTGTACTACCCCGACAAGGTGTTCAGATCCAGCGTGCTGCACTCTACCCAGGACCTGTTCCTGCCTTTC
TTCAGCAACGTGACCTGGTTCCACGCCATCCACGTGTCCGGCACCAATGGCACCAAGAGATTCGACAACCCCGTGCTGCCCTTCAAC
GACGGGGTGTACTTTGCCAGCACCGAGAAGTCCAACATCATCAGAGGCTGGATCTTCGGCACCACACTGGACAGCAAGACCCAGAGC
CTGCTGATCGTGAACAACGCCACCAACGTGGTCATCAAAGTGTGCGAGTTCCAGTTCTGCAACGACCCCTTCCTGGGCGTCTACTAC
CACAAGAACAACAAGAGCTGGATGGAAAGCGAGTTCCGGGTGTACAGCAGCGCCAACAACTGCACCTTCGAGTACGTGTCCCAGCCT
TTCCTGATGGACCTGGAAGGCAAGCAGGGCAACTTCAAGAACCTGCGCGAGTTCGTGTTCAAGAACATCGACGGCTACTTCAAGATC
TACAGCAAGCACACCCCTATCAACCTCGTGCGGGATCTGCCTCAGGGCTTCTCTGCTCTGGAACCCCTGGTGGATCTGCCCATCGGC
ATCAACATCACCCGGTTTCAGACACTGCTGGCCCTGCACAGAAGCTACCTGACACCTGGCGATAGCAGCAGCGGATGGACAGCTGGT
GCCGCCGCTTACTATGTGGGCTACCTGCAGCCTAGAACCTTCCTGCTGAAGTACAACGAGAACGGCACCATCACCGACGCCGTGGAT
TGTGCTCTGGATCCTCTGAGCGAGACAAAGTGCACCCTGAAGTCCTTCACCGTGGAAAAGGGCATCTACCAGACCAGCAACTTCCGG
GTGCAGCCCACCGAATCCATCGTGCGGTTCCCCAATATCACCAATCTGTGCCCCTTCGGCGAGGTGTTCAATGCCACCAGATTCGCC
TCTGTGTACGCCTGGAACCGGAAGCGGATCAGCAATTGCGTGGCCGACTACTCCGTGCTGTACAACTCCGCCAGCTTCAGCACCTTC
AAGTGCTACGGCGTGTCCCCTACCAAGCTGAACGACCTGTGCTTCACAAACGTGTACGCCGACAGCTTCGTGATCCGGGGAGATGAA
GTGCGGCAGATTGCCCCTGGACAGACAGGCAAGATCGCCGACTACAACTACAAGCTGCCCGACGACTTCACCGGCTGTGTGATTGCC
TGGAACAGCAACAACCTGGACTCCAAAGTCGGCGGCAACTACAATTACCTGTACCGGCTGTTCCGGAAGTCCAATCTGAAGCCCTTC
GAGCGGGACATCTCCACCGAGATCTATCAGGCCGGCAGCACCCCTTGTAACGGCGTGGAAGGCTTCAACTGCTACTTCCCACTGCAG
TCCTACGGCTTTCAGCCCACAAATGGCGTGGGCTATCAGCCCTACAGAGTGGTGGTGCTGAGCTTCGAACTGCTGCATGCCCCTGCC
ACAGTGTGCGGCCCTAAGAAAAGCACCAATCTCGTGAAGAACAAATGCGTGAACTTCAACTTCAACGGCCTGACCGGCACCGGCGTG
CTGACAGAGAGCAACAAGAAGTTCCTGCCATTCCAGCAGTTTGGCCGGGATATCGCCGATACCACAGACGCCGTTAGAGATCCCCAG
ACACTGGAAATCCTGGACATCACCCCTTGCAGCTTCGGCGGAGTGTCTGTGATCACCCCTGGCACCAACACCAGCAATCAGGTGGCA
GTGCTGTACCAGGACGTGAACTGTACCGAAGTGCCCGTGGCCATTCACGCCGATCAGCTGACACCTACATGGCGGGTGTACTCCACC
GGCAGCAATGTGTTTCAGACCAGAGCCGGCTGTCTGATCGGAGCCGAGCACGTGAACAATAGCTACGAGTGCGACATCCCCATCGGC
GCTGGCATCTGTGCCAGCTACCAGACACAGACAAACAGCCCTGGCAGCGCCTCTTCTGTGGCCAGCCAGAGCATCATTGCCTACACA
ATGTCTCTGGGCGCCGAGAACAGCGTGGCCTACTCCAACAACTCTATCGCTATCCCCACCAACTTCACCATCAGCGTGACCACAGAG
ATCCTGCCTGTGTCCATGACCAAGACCAGCGTGGACTGCACCATGTACATCTGCGGCGATTCCACCGAGTGCTCCAACCTGCTGCTG
CAGTACGGCAGCTTCTGCACCCAGCTGAATAGAGCCCTGACAGGGATCGCCGTGGAACAGGACAAGAACACCCAAGAGGTGTTCGCC
CAAGTGAAGCAGATCTACAAGACCCCTCCTATCAAGGACTTCGGCGGCTTCAATTTCAGCCAGATTCTGCCCGATCCTAGCAAGCCC
AGCAAGCGGAGCTTCATCGAGGACCTGCTGTTCAACAAAGTGACACTGGCCGACGCCGGCTTCATCAAGCAGTATGGCGATTGTCTG
GGCGACATTGCCGCCAGGGATCTGATTTGCGCCCAGAAGTTTAACGGACTGACAGTGCTGCCTCCTCTGCTGACCGATGAGATGATC
GCCCAGTACACATCTGCCCTGCTGGCCGGCACAATCACAAGCGGCTGGACATTTGGAGCTGGCGCCGCTCTGCAGATCCCCTTTGCT
ATGCAGATGGCCTACAGGTTCAACGGCATCGGAGTGACCCAGAATGTGCTGTACGAGAACCAGAAGCTGATCGCCAACCAGTTCAAC
AGCGCCATCGGCAAGATCCAGGACAGCCTGAGCAGCACAGCAAGCGCCCTGGGAAAGCTGCAGGACGTGGTCAACCAGAATGCCCAG
GCACTGAACACCCTGGTCAAGCAGCTGTCCTCCAACTTCGGCGCCATCAGCTCTGTGCTGAACGATATCCTGAGCAGACTGGACCCT
CCTGAGGCCGAGGTGCAGATCGACAGACTGATCACAGGCAGACTGCAGAGCCTCCAGACATACGTGACCCAGCAGCTGATCAGAGCC
GCCGAGATTAGAGCCTCTGCCAATCTGGCCGCCACCAAGATGTCTGAGTGTGTGCTGGGCCAGAGCAAGAGAGTGGACTTTTGCGGC
AAGGGCTACCACCTGATGAGCTTCCCTCAGTCTGCCCCTCACGGCGTGGTGTTTCTGCACGTGACATATGTGCCCGCTCAAGAGAAG
AATTTCACCACCGCTCCAGCCATCTGCCACGACGGCAAAGCCCACTTTCCTAGAGAAGGCGTGTTCGTGTCCAACGGCACCCATTGG
TTCGTGACACAGCGGAACTTCTACGAGCCCCAGATCATCACCACCGACAACACCTTCGTGTCTGGCAACTGCGACGTCGTGATCGGC
ATTGTGAACAATACCGTGTACGACCCTCTGCAGCCCGAGCTGGACAGCTTCAAAGAGGAACTGGACAAGTACTTTAAGAACCACACA
AGCCCCGACGTGGACCTGGGCGATATCAGCGGAATCAATGCCAGCGTCGTGAACATCCAGAAAGAGATCGACCGGCTGAACGAGGTG
GCCAAGAATCTGAACGAGAGCCTGATCGACCTGCAAGAACTGGGGAAGTACGAGCAAGGCTCTGGAATTGTGCAGCAGCAAAACAAT
CTGCTGCGGGCCATCGAGGCTCAGCAGCATCTGCTGCAGCTGACCGTGTGGGGAATCAAGCAGCTCCAGGCCAGAATCCTGGCTGGT
GGATCTGGCGGCCATACCACCTGGATGGAATGGGACAGAGAGATCAACAACTACACCAGCCTGATCCACAGCCTGATTGAGGAATCC
CAGAATCAGCAAGAGAAGAACGAGCAAGAACTGCTGGAAGGAAGCGGAGGCTCTGGTaaactgggctctattgaatttattaaagtg
aacaaaGAGCCCGAAGCCTAA
SARS CoV2 Spike RBD-SnoopTagJr
Amino acid (SEQ ID NO: 68):
MFVFLVLLPLVSSQCRVQPTESIVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKSTNGSGGSGKLGSIEFIKVNKEPEA
DNA (incl. STOP) (SEQ ID NO: 69):
ATGTTCGTGTTTCTGGTGCTGCTGCCTCTGGTGTCCAGCCAGTGTCGGGTGCAGCCCACCGAATCCATCGTGCGGTTCCCCAATATC
ACCAATCTGTGCCCCTTCGGCGAGGTGTTCAATGCCACCAGATTCGCCTCTGTGTACGCCTGGAACCGGAAGCGGATCAGCAATTGC
GTGGCCGACTACTCCGTGCTGTACAACTCCGCCAGCTTCAGCACCTTCAAGTGCTACGGCGTGTCCCCTACCAAGCTGAACGACCTG
TGCTTCACAAACGTGTACGCCGACAGCTTCGTGATCCGGGGAGATGAAGTGCGGCAGATTGCCCCTGGACAGACAGGCAAGATCGCC
GACTACAACTACAAGCTGCCCGACGACTTCACCGGCTGTGTGATTGCCTGGAACAGCAACAACCTGGACTCCAAAGTCGGCGGCAAC
TACAATTACCTGTACCGGCTGTTCCGGAAGTCCAATCTGAAGCCCTTCGAGCGGGACATCTCCACCGAGATCTATCAGGCCGGCAGC
ACCCCTTGTAACGGCGTGGAAGGCTTCAACTGCTACTTCCCACTGCAGTCCTACGGCTTTCAGCCCACAAATGGCGTGGGCTATCAG
CCCTACAGAGTGGTGGTGCTGAGCTTCGAACTGCTGCATGCCCCTGCCACAGTGTGCGGCCCTAAGAAAAGCACCAATGGAAGCGGA
GGCTCTGGTaaactgggctctattgaatttattaaagtgaacaaaGAGCCCGAAGCCTAA
SARS CoV2 Spike RBD-SnoopCatcher
Amino acid (SEQ ID NO: 70):
MFVFLVLLPLVSSQCRVQPTESIVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKSTNGSGGSGGSGGSGHMKPLRGAVFSLQKQHPDYP
DIYGAIDQNGTYQNVRTGEDGKLTFKNLSDGKYRLFENSEPAGYKPVQNKPIVAFQIVNGEVRDVTSIVPQDIPATYEFTNGKHYIT
NEPIPPKEPEA
DNA (incl. STOP) (SEQ ID NO: 71):
ATGTTCGTGTTTCTGGTGCTGCTGCCTCTGGTGTCCAGCCAGTGTCGGGTGCAGCCCACCGAATCCATCGTGCGGTTCCCCAATATC
ACCAATCTGTGCCCCTTCGGCGAGGTGTTCAATGCCACCAGATTCGCCTCTGTGTACGCCTGGAACCGGAAGCGGATCAGCAATTGC
GTGGCCGACTACTCCGTGCTGTACAACTCCGCCAGCTTCAGCACCTTCAAGTGCTACGGCGTGTCCCCTACCAAGCTGAACGACCTG
TGCTTCACAAACGTGTACGCCGACAGCTTCGTGATCCGGGGAGATGAAGTGCGGCAGATTGCCCCTGGACAGACAGGCAAGATCGCC
GACTACAACTACAAGCTGCCCGACGACTTCACCGGCTGTGTGATTGCCTGGAACAGCAACAACCTGGACTCCAAAGTCGGCGGCAAC
TACAATTACCTGTACCGGCTGTTCCGGAAGTCCAATCTGAAGCCCTTCGAGCGGGACATCTCCACCGAGATCTATCAGGCCGGCAGC
ACCCCTTGTAACGGCGTGGAAGGCTTCAACTGCTACTTCCCACTGCAGTCCTACGGCTTTCAGCCCACAAATGGCGTGGGCTATCAG
CCCTACAGAGTGGTGGTGCTGAGCTTCGAACTGCTGCATGCCCCTGCCACAGTGTGCGGCCCTAAGAAAAGCACCAATGGAAGCGGA
GGCTCTGGTGGATCCGGTGGATCTGGACACATGAAGCCTCTGAGAGGCGCCGTGTTCAGCCTGCAGAAACAGCACCCTGACTACCCC
GATATCTACGGCGCCATCGACCAGAACGGCACCTACCAGAATGTTCGGACAGGCGAGGATGGCAAGCTGACCTTCAAGAACCTGAGC
GACGGCAAGTACCGGCTGTTCGAGAATTCTGAGCCTGCCGGCTACAAGCCCGTGCAGAACAAACCTATCGTGGCCTTCCAGATCGTG
AACGGCGAAGTGCGGGATGTGACCAGCATCGTGCCTCAGGATATCCCCGCCACCTACGAGTTCACCAACGGCAAGCACTACATCACC
AACGAGCCCATTCCTCCAAAAGAGCCCGAAGCCTAA
SnoopCatcher-SARS CoV2 Spike RBD
Amino acid (SEQ ID NO: 72):
METDTLLLWVLLLWVPGSTGDGHMKPLRGAVFSLQKQHPDYPDIYGAIDQNGTYQNVRTGEDGKLTFKNLSDGKYRLFENSEPAGYK
PVQNKPIVAFQIVNGEVRDVTSIVPQDIPATYEFTNGKHYITNEPIPPKGSGGSGGSRVQPTESIVRFPNITNLCPFGEVFNATRFA
SVYAWNRKRISNCVADYSVLYNSASFSTFKCYGVSPTKLNDLCFTNVYADSFVIRGDEVRQIAPGQTGKIADYNYKLPDDFTGCVIA
WNSNNLDSKVGGNYNYLYRLFRKSNLKPFERDISTEIYQAGSTPCNGVEGFNCYFPLQSYGFQPTNGVGYQPYRVVVLSFELLHAPA
TVCGPKKSTNEPEA
DNA (incl. STOP) (SEQ ID NO: 73):
atggagacagacacactcctgctatgggtactgctgctctgggttccaggttccactggtgacGGTCACATGAAGCCCCTGAGGGGC
GCCGTGTTCAGCCTGCAGAAGCAGCACCCCGACTACCCCGACATCTACGGCGCCATCGACCAGAACGGCACCTACCAGAACGTGAGG
ACCGGCGAGGACGGCAAGCTGACCTTCAAGAACCTGAGCGACGGCAAGTACAGGCTGTTCGAGAACAGCGAGCCCGCCGGCTACAAG
CCCGTGCAGAACAAGCCCATCGTGGCCTTCCAGATCGTGAACGGCGAGGTGAGGGACGTGACCAGCATCGTGCCCCAGGACATCCCC
GCCACCTACGAGTTCACCAACGGCAAGCACTACATCACCAACGAGCCCATCCCCCCCAAGGGCTCTGGCGGAAGCGGCggatccCGG
GTGCAGCCCACCGAATCCATCGTGCGGTTCCCCAATATCACCAATCTGTGCCCCTTCGGCGAGGTGTTCAATGCCACCAGATTCGCC
TCTGTGTACGCCTGGAACCGGAAGCGGATCAGCAATTGCGTGGCCGACTACTCCGTGCTGTACAACTCCGCCAGCTTCAGCACCTTC
AAGTGCTACGGCGTGTCCCCTACCAAGCTGAACGACCTGTGCTTCACAAACGTGTACGCCGACAGCTTCGTGATCCGGGGAGATGAA
GTGCGGCAGATTGCCCCTGGACAGACAGGCAAGATCGCCGACTACAACTACAAGCTGCCCGACGACTTCACCGGCTGTGTGATTGCC
TGGAACAGCAACAACCTGGACTCCAAAGTCGGCGGCAACTACAATTACCTGTACCGGCTGTTCCGGAAGTCCAATCTGAAGCCCTTC
GAGCGGGACATCTCCACCGAGATCTATCAGGCCGGCAGCACCCCTTGTAACGGCGTGGAAGGCTTCAACTGCTACTTCCCACTGCAG
TCCTACGGCTTTCAGCCCACAAATGGCGTGGGCTATCAGCCCTACAGAGTGGTGGTGCTGAGCTTCGAACTGCTGCATGCCCCTGCC
ACAGTGTGCGGCCCTAAGAAAAGCACCAATGAGCCCGAGGCCTAA
SARS CoV2 Spike RBD-SpyCatcher
Amino acid (SEQ ID NO: 74):
MFVFLVLLPLVSSQCRVQPTESIVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKKSTNGSGGSGGSGGSGDSATHIKFSKRDEDGKELAG
ATMELRDSSGKTISTWISDGQVKDFYLYPGKYTFVETAAPDGYEVATAITFTVNEQGQVTVNGKATKGDAHIEPEA
DNA (incl. STOP) (SEQ ID NO: 75):
ATGTTCGTGTTTCTGGTGCTGCTGCCTCTGGTGTCCAGCCAGTGTCGGGTGCAGCCCACCGAATCCATCGTGCGGTTCCCCAATATC
ACCAATCTGTGCCCCTTCGGCGAGGTGTTCAATGCCACCAGATTCGCCTCTGTGTACGCCTGGAACCGGAAGCGGATCAGCAATTGC
GTGGCCGACTACTCCGTGCTGTACAACTCCGCCAGCTTCAGCACCTTCAAGTGCTACGGCGTGTCCCCTACCAAGCTGAACGACCTG
TGCTTCACAAACGTGTACGCCGACAGCTTCGTGATCCGGGGAGATGAAGTGCGGCAGATTGCCCCTGGACAGACAGGCAAGATCGCC
GACTACAACTACAAGCTGCCCGACGACTTCACCGGCTGTGTGATTGCCTGGAACAGCAACAACCTGGACTCCAAAGTCGGCGGCAAC
TACAATTACCTGTACCGGCTGTTCCGGAAGTCCAATCTGAAGCCCTTCGAGCGGGACATCTCCACCGAGATCTATCAGGCCGGCAGC
ACCCCTTGTAACGGCGTGGAAGGCTTCAACTGCTACTTCCCACTGCAGTCCTACGGCTTTCAGCCCACAAATGGCGTGGGCTATCAG
CCCTACAGAGTGGTGGTGCTGAGCTTCGAACTGCTGCATGCCCCTGCCACAGTGTGCGGCCCTAAGAAAAGCACCAATGGAAGCGGA
GGCTCTGGTGGATCCGGTGGATCTGGCGATTCTGCCACACACATCAAGTTCAGCAAGCGCGACGAGGACGGCAAAGAACTGGCTGGC
GCTACCATGGAACTGAGAGACAGCAGCGGCAAGACCATCAGCACCTGGATCTCCGACGGCCAAGTGAAGGACTTCTATCTGTACCCC
GGCAAGTACACCTTCGTGGAAACCGCCGCTCCTGACGGATATGAAGTGGCCACCGCCATCACCTTCACCGTGAATGAGCAGGGACAA
GTGACCGTGAACGGCAAGGCCACAAAAGGCGACGCCCACATTGAGCCCGAAGCCTAA

### Example 1—Modular Covalent Decoration of the Adenovirus Capsid Via Insertion of SpyTag or DogTag into Hexon HVR Loops

In order to modify the hexon proteins of the adenovirus capsid, sequence alignment was undertaken to identify the location of hypervariable region (HVR) loops 1, 2 and 5 in FIG. 1E [Sequence comparison of the following adenoviruses; Ad5 and Ad2 (species C), Ad12 and Ad18 (species A), Ad3 and Ad35 (species B), Ad4 (species E), Ad40 and Ad41 (species F)]. Deletions were generated at the insertion sites of the HVR loops indicated in FIG. 1E. SpyTag and DogTag were inserted at each locus. The insertion sites indicated are identical between SpyTag and DogTag recombinants at each of the three HVRs tested. At each locus, SpyTag or DogTag sequences are flanked by GSGGSG sequences on each side.

### Example 2—Coupling of SpyCatcher with SpyTag Inserted into HVR Loops

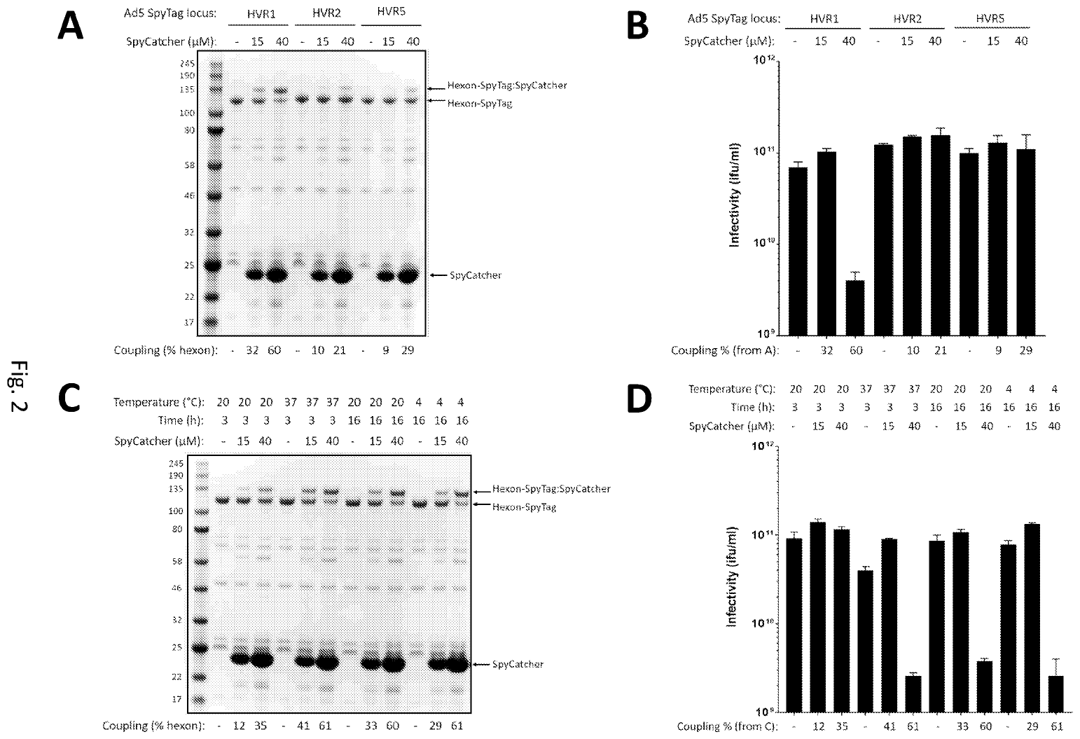

Ad5 vectors displaying SpyTag at HVR1, HVR2 or HVR5 were incubated with biotinylated SpyCatcher at 15 μM or 40 μM in order to assess the coupling reaction between SpyTag and SpyCatcher. Samples were run on SDS-PAGE and proteins visualised by Coomassie staining as shown in FIG. 2A.Coupling efficiency was assessed by comparing band intensities of uncoupled hexon-SpyTag with hexon-SpyTag:SpyCatcher using ImageJ software (% hexon coupled=band intensity of hexon-SpyTag:SpyCatcher divided by the sum of band intensities of hexon-SpyTag:SpyCatcher and uncoupled hexon-SpyTag, multiplied by 100).

Immediately after the coupling reaction described in FIG. 2A, a vector infectivity assay was undertaken with the same samples (FIG. 2B). Samples were serially diluted, applied to monolayers of HEK293A cells in a 96-well plate, and incubated for 48 h at 37° C. with 5% CO₂. Infectious titres were calculated by enumeration of GFP fluorescent foci by fluorescence microscopy. Infectious titres were calculated as no. of infectious units (ifu) per ml.

Ad5(GFP) vectors displaying SpyTag at HVR1 (Ad5(GFP) HVR1 SpyTag) (1E+10 viral particles) were incubated with biotinylated SpyCatcher at 15 μM or 40 μM under different conditions (FIG. 2C). Samples were run on SDS-PAGE and proteins visualised by Coomassie staining. A vector infectivity assay was performed on the same samples as shown in FIG. 2C (FIG. 2D).

The results demonstrate that SpyTag is poorly reactive after insertion into Ad5 HVR loops, and that vector infectivity is substantially reduced (over 100-fold) upon coupling of SpyCatcher to >50% of hexon protein on the viral capsid.

### Example 3—Coupling of DogCatcher with DogTag Inserted into HVR Loops

HEK293A cells were infected either with wild type Ad5(GFP) (native hexon protein) or DogTag inserted into HVR1 (HVR1DT), HVR2 (HVR2DT) or HVR5 (HVR5DT). Vector yield from 1500 cm² of adherent HEK293A cells infected with Ad5 displaying DogTag on hexon surfaces is equivalent to vector yield with unmodified hexon Ad5 (FIG. 3A).

Ad5(GFP) vectors (1E+10 viral particles) displaying DogTag at either HVR1, HVR2 or HVR5 were incubated with 5 μM DogCatcher. Reactions were performed for 16 h at 4° C. as shown in FIG. 3B. Coupling efficiency was assessed by comparing band intensities of uncoupled hexon-DogTag with hexon-DogTag:DogCatcher using ImageJ as described in Example 2. FIG. 3C shows an infectivity assay performed on the same samples as in FIG. 3B.

Ad5(GFP) vectors displaying DogTag at either HVR1, HVR2 or HVR5 (1E+10 viral particles) were incubated with DogCatcher at 20 μM or 80 μM for 0.1, 1 or 16 hours at 4° C. as shown in FIG. 3D. Coupling efficiency was assessed as described above.

The data show that DogTag is highly reactive after insertion into Ad5 HVR loops and that vector infectivity is not inhibited by coupling to DogCatcher, despite coverage of >90% capsid hexon.

### Example 4—Reactivity of SnoopTagJr with DogTag Inserted into HVR Loops

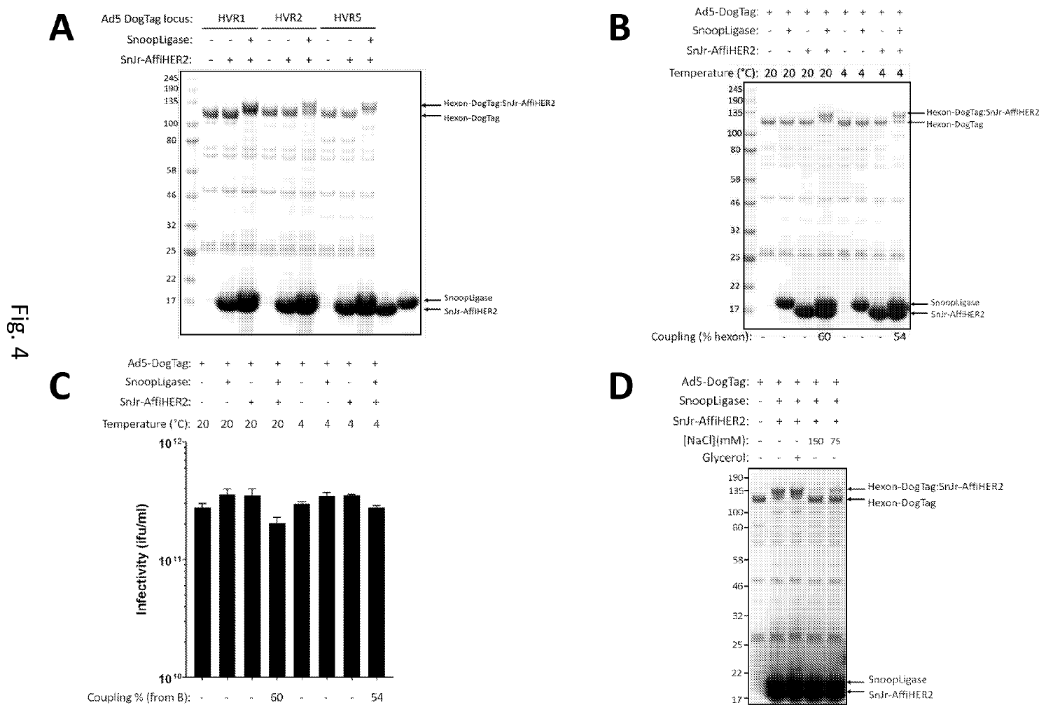

Ad5(GFP) vectors (1E+10 viral particles) displaying DogTag at either HVR1, HVR2 or HVR5 were incubated with SnoopTagJr-AffiHER2 (80 μM) and SnoopLigase (70 μM). Samples were run on SDS-PAGE and proteins visualised by Coomassie staining. Coupling efficiency was quantified by comparing band intensities of uncoupled hexon-DogTag with hexon-DogTag:SnJr-AffiHER2 using ImageJ (FIG. 4A).

To assess the temperature-dependence of hexon-DogTag coupling to SnoopTagJr-AffiHER2, Ad5(GFP) vectors displaying DogTag at HVR5 (1E+10 viral particles) were incubated with SnoopTagJr-AffiHER2 (80 μM) and SnoopLigase (70 μM) for 16 h at either 20° C. or 4° C. (FIG. 4B). A vector infectivity assay was performed as described in Example 3 on the samples used for FIG. 4B (FIG. 4C).

Ad5(GFP) vectors displaying DogTag at HVR5 (1E+10 viral particles) were incubated with SnoopTagJr-AffiHER2 (80 μM) and SnoopLigase (70 μM) for 16 h at 4° C. (FIG. 4D). Different concentrations of NaCl were added at either 150 mM or 75 mM, and glycerol was added as indicated at 15% (v/v). Samples were analysed by SDS-PAGE and Coomassie staining.

### Example 5—Assessment of Vector Infectivity for Non-Fluorescent Recombinant Vectors

A hexon immunostaining assay to assess vector infectivity was performed with Ad5(GFP) (with a WT hexon) and Ad5(GFP) HVR5 DogTag. Both vectors were serially diluted, and 50 μl of each dilution was transferred to a monolayer of HEK293A cells in a poly-L-lysine coated 96-well plate. After a 48 h incubation at 37° C. with 5% CO₂, GFP-positive cells were counted by fluorescence microscopy. For immunostaining, cells on the same plate were fixed with ice-cold methanol and an anti-hexon mouse monoclonal antibody (clone 65H6, ThermoFisher) was used to detect hexon protein within infected cells. Hexon-positive cells were subsequently stained using an alkaline phosphatase-conjugated secondary antibody, and BCIP/NBT substrate solution. The results demonstrated that HEK293A cells infected with Ad5 vectors with a WT hexon protein and Ad5 vectors displaying DogTag at HVR5 showed equivalent intensity of hexon immunostaining.

The infectious titres for Ad5(GFP) (hexon WT) and Ad5(GFP) HVR5 DogTag were compared using both GFP fluorescence and hexon immunostaining assays. Both assays were performed using the same wells on the same plate, n=4. Bars show mean+SD and dots represent spot counts from individual wells. The results were not significantly different (ns) as calculated by a two-tailed t-test.

### Example 6—Reactivity of DogTag Inserted into Hexon Loops with SnoopTagJr Tagged Peptides

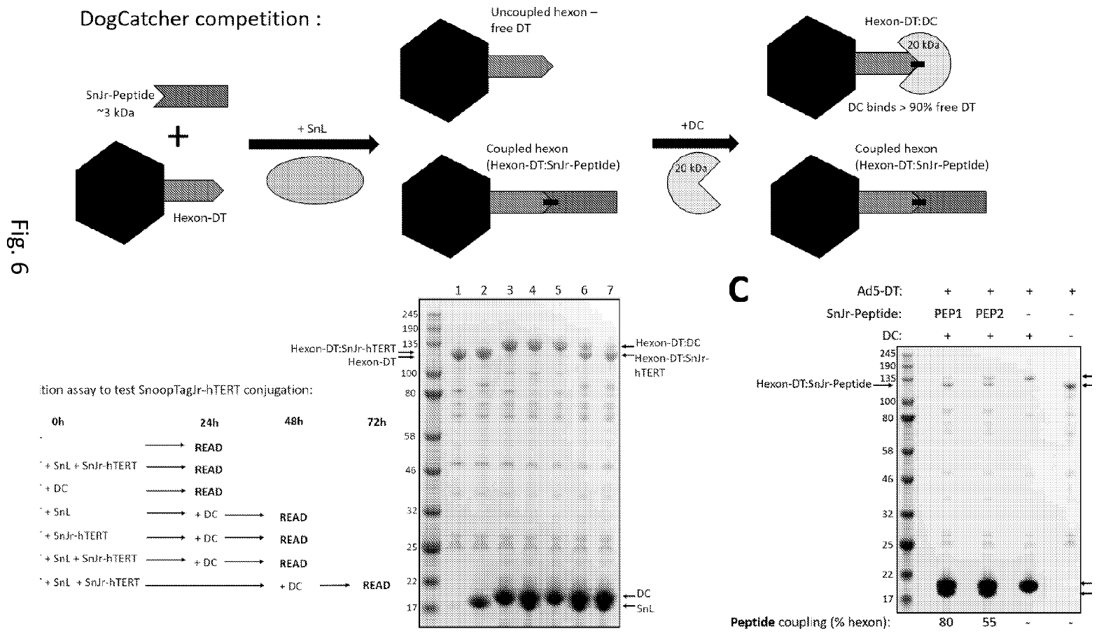

Peptides were synthesized consisting of the SnoopTagJr sequence fused to T cell epitopes. These SnoopTagJr tagged peptides were coupled to Ad5(GFP) HVR5 DogTag using SnoopLigase. A gel shift assay with Coomassie staining was not suitable to assess the coupling efficiency due to the low molecular weight of these short peptides (^{˜}3 kDa). The efficiency of peptide coupling was instead assessed using a competition assay with DogCatcher (described in FIG. 6A). SnoopTagJr-peptide (SnJr-peptide) and hexon-DogTag (hexon-DT) are coupled using SnoopLigase (SnL). After coupling, an excess of DogCatcher (DC) protein is added to the reaction. DC binds >90% of free (uncoupled) hexon-DT molecules on the surface of Ad5 (see FIG. 3B), therefore the proportion of hexon-DT coupled to SnJr-peptide can be estimated to be the proportion of hexon that does not undergo a gel shift of ^{˜}20 kDa on SDS-PAGE after incubation with DC.

Coupling efficiency of SnoopTagJr-hTERT peptide (SnoopTagJr fused to a T cell epitope from human telomerase reverse transcriptase, EARPALLTSRLRFIPK) to hexon-DT was assessed using the DogCatcher competition assay described above. Ad5(GFP) HVR5 DogTag (Ad5-DT, 1E+10 viral particles) was incubated with different combinations of SnL (70 μM) and SnJr-hTERT (80 μM), and in some samples DogCatcher (20 μM, excess) was added after the peptide-peptide coupling reaction to assess coupling efficiency (FIG. 6B). All reactions were performed at 4° C. Samples were run on SDS-PAGE with subsequent Coomassie staining (FIG. 6B). The proportion of coupled hexon-DT:SnJr-hTERT compared to hexon-DT:DC (representing free hexon-DT after the initial SnL-catalysed reaction) was assessed by comparing band intensities as indicated. The optimum time interval for the hexon-DT:SnJr-hTERT reaction was determined to be 48 h.

FIG. 6C shows an assessment of coupling efficiency of SnoopTagJr-SIINFEKL peptides (SIINFEKL is a mouse CD8⁺ T cell epitope derived from ovalbumin) to hexon-DT using the DogCatcher competition assay described above. PEP1 corresponds to the peptide sequence SnJr-GGSSIINFEKL and PEP2 corresponds to the peptide sequence SnJr-AAYSIINFEKL. Ad5-DT (1E+10 viral particles) was incubated with SnJr-SIINFEKL peptides (PEP1 or PEP2, 80 μM) and SnL (70 μM) for 48 h at 4° C. DC (20 μM) was subsequently added. Samples were then incubated for a further 24 h at 4° C. Samples were run on SDS-PAGE and peptide coupling efficiency was assessed as described above.

Alternatively, a direct gel shift assay using monovalent streptavidin (mSA) can be used to assess coupling efficiency of short peptides as described in FIG. 6D. Biotinylated SnJr-peptide was incubated with SnL and Ad5-DT as described previously. After 48 h, the coupling reaction was stopped by boiling in sample buffer including sodium dodecyl sulfate (SDS) and dithiothreitol (DTT) to denature all proteins and virion structures. Samples were briefly cooled on ice before incubation with an excess of monovalent streptavidin (mSA) for 30 min at 20° C. Samples were run on SDS-PAGE, transferred to nitrocellulose, and Western blotting performed using an anti-hexon primary mouse monoclonal antibody (clone 65H6, ThermoFisher). A gel-shift (^{˜}50 kDa) is observed for hexon-DT protein coupled to SnJr-biotin by virtue of the strong interaction between mSA and biotin (hexon-DT:SnJr-biotin/mSA). FIG. 6E shows the SDS-PAGE of the mSA direct gel shift assay as described above with additional controls. Bands corresponding to hexon-DT:SnJr-biotin/mSA and hexon-DT species are indicated. FIG. 6F shows a vector infectivity assay (by GFP focus enumeration, as described in example 5) was performed on the same samples shown in FIG. 6C. The results demonstrate that Ad5-DT infectivity is not affected by surface display of SnJr-peptides.

### Example 7—Coupling of Ad5 HVR DogTag to DogCatcher Fusion Proteins

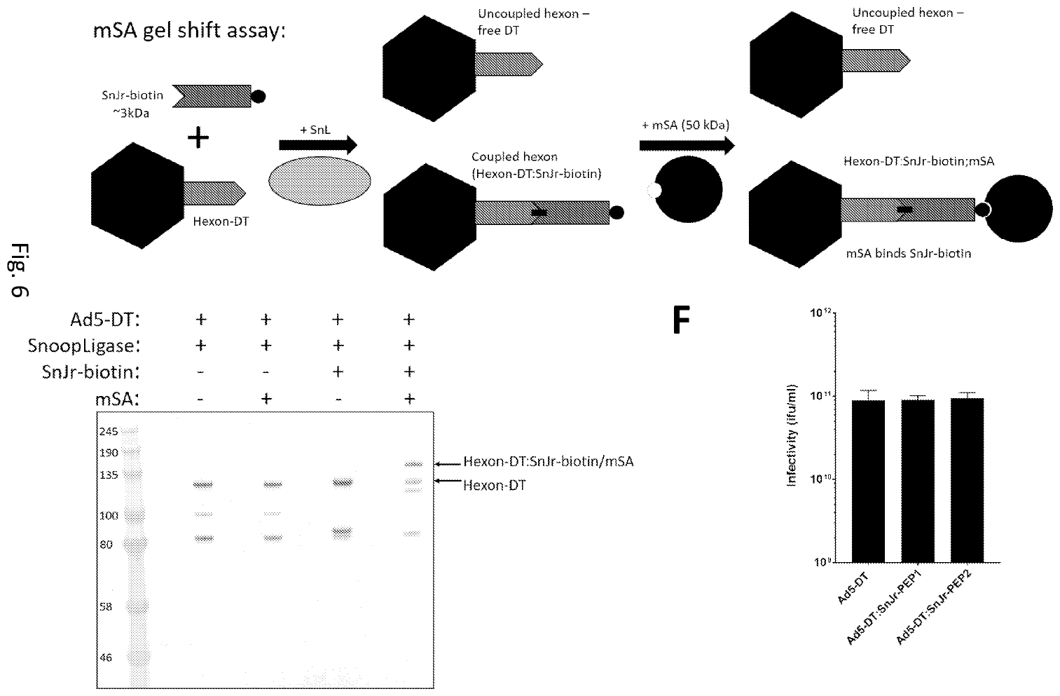

The circumsporozoite protein (CSP) of Plasmodium falciparum (Pf) has been extensively studied as a malaria vaccine candidate antigen. The protein contains a highly immunogenic repetitive region, primarily consisting of repeats of the sequence NANP. Repeat sequences of varying lengths from this region were fused to the C-terminus of DogCatcher and the resulting fusion proteins coupled to Ad5(GFP) HVR5 DogTag. Three DogCatcher fusion constructs; NANP9, NANP18, and NANPD (consisting of the entire NANP repetitive domain of PfCSP from the 3D7 strain) are shown in FIG. 7A.

Reactivity of DogCatcher-NANPn with DogTag inserted into hexon HVR5 loop was assessed. Ad5(GFP) vectors (1E+9 viral particles) displaying DogTag at HVR5 were incubated with different DogCatcher-NANPn fusions (as indicated) at 15 μM. Reactions were performed for 16 h at 4° C. Samples were run on SDS-PAGE and proteins visualised by Coomassie staining (FIG. 7B). Coupling efficiency was assessed as described above. A vector infectivity assay (by GFP focus enumeration, as described above) was performed on the same samples shown in FIG. 7B (FIG. 7C).

### Example 8—Coupling of DogCatcher-NANPn to the Surface of Ad5 Via Hexon Reduces the Potency of Virus Neutralising Antibodies

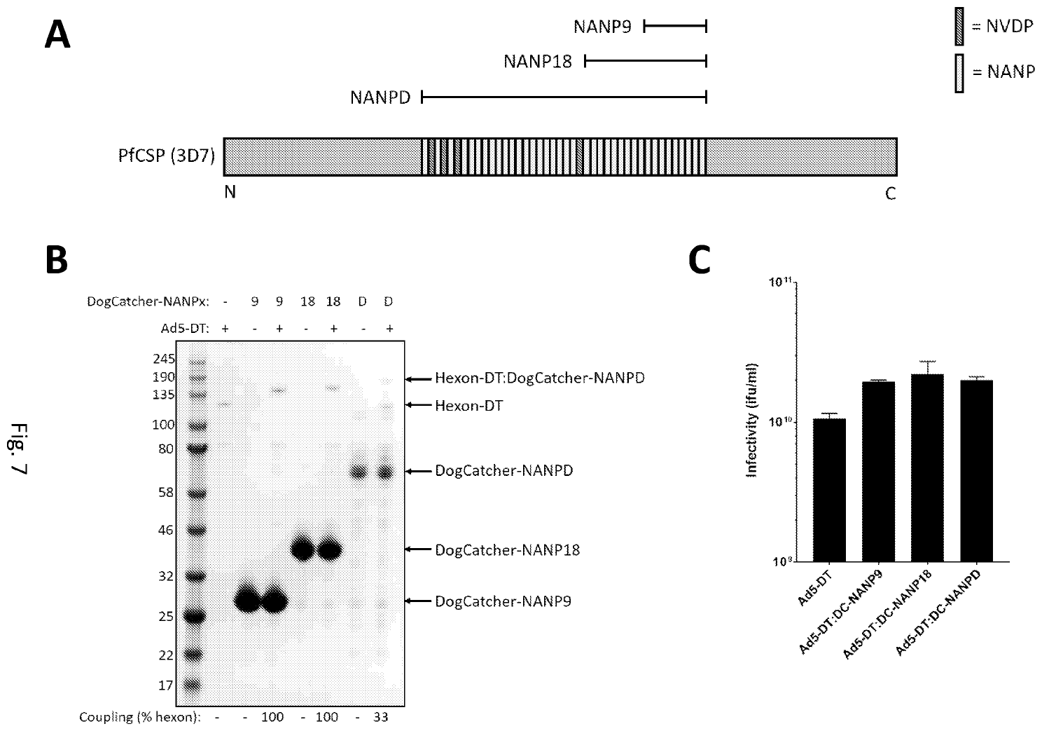

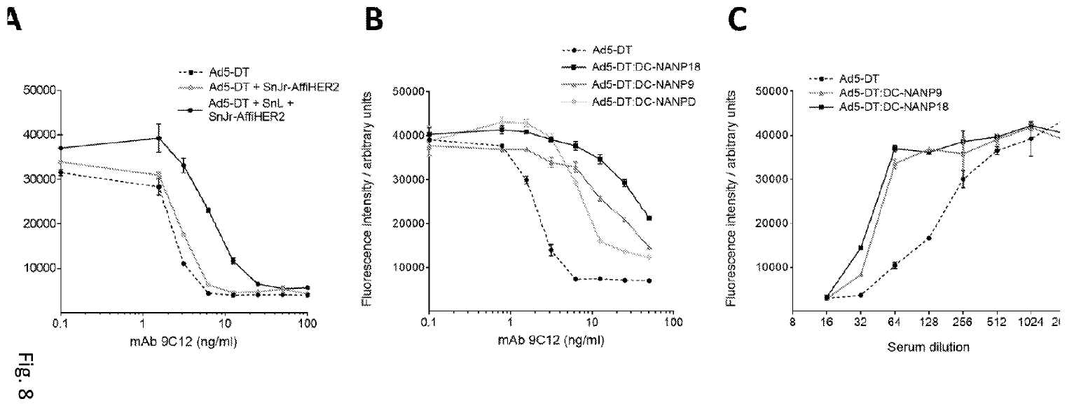

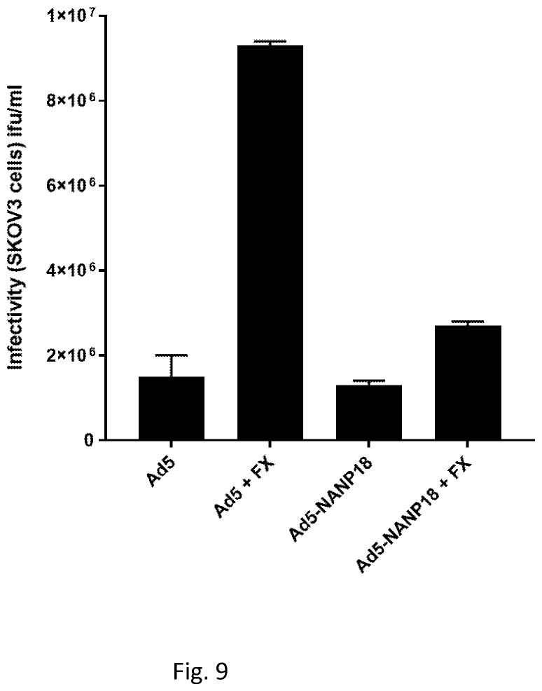

Ad5(GFP) HVR5 DogTag vectors (Ad5-DT, 1E+10 viral particles) were incubated with SnoopLigase (SnL) and SnJr-AffiHER2 (or SnJr-AffiHER2 alone) for 48 h at 4° C. After 48 h, approximately 60-65% of the hexon was coupled to SnJr-AffiHER2 (a comparable coupling efficiency to the experiment shown in FIG. 4B). Coupled vector was incubated with serially diluted virus-neutralising mouse monoclonal anti-hexon antibody mAb 9C12 (Developmental Studies Hybridoma Bank, University of Iowa) for 1 h at 37° C., before the vector-antibody mix was added to an 80% confluent monolayer of HEK293A cells in a 96-well plate format (cells were infected at a multiplicity of infection of 200 ifu/cell). Cells were incubated with the vector-antibody mix for 2 h at 37° C. with 5% CO₂, before the mix was replaced with fresh media and the plates returned to 37° C. with 5% CO₂ for a further 24 h. After 24 h, GFP expression within HEK293A cells was employed as a readout of infectivity—bulk fluorescence was measured on a fluorimeter using an excitation of 395 nm and emission of 509 nm. The data, shown in FIG. 8A, demonstrated an increase in the IC₅₀ of a potent neutralising anti-hexon monoclonal antibody upon coupling of SnoopTagJr-AffiHER2 to Ad5 DogTag. IC₅₀ for mAb 9C12 neutralisation increased from 2.5 ng/ml (Ad5-DT alone, and Ad5-DT+SnJr-AffiHER2 only) to 7 ng/ml with coupled vector (Ad5-DT+SnL+SnJr-AffiHER2).

In FIG. 8B, a similar experiment to that described above was performed using the DogCatcher-NANPn fusions described in FIG. 7A. Ad5(GFP) HVR5 DogTag vectors (Ad5-DT, 1E+9 viral particles) were incubated with DogCatcher-NANPn constructs for 16 h at 4° C., exactly as described in FIG. 7. Coupling efficiency of each of the constructs to Ad5-DT was similar to that shown in FIG. 7B. As described above, vector was incubated with mAb 9C12 for 1 h, the mix added to cells at a multiplicity of infection of 200 ifu/cell for 2 h, and vector replaced with fresh media prior to reading GFP fluorescence after a further 24 h. A substantial increase in IC₅₀ for mAb 9C12 was observed with the DogCatcher-NANPn coupled vectors compared to Ad5-DT alone, implying shielding of the hexon from the neutralising monoclonal antibody (FIG. 8B). Ad5-DT:DC-NANP18 exhibited the greatest increase in IC₅₀, >10-fold compared to Ad5-DT alone.

In FIG. 8C, a neutralisation assay with Ad5-positive mouse serum (instead of mAb 9C12) was performed on coupled Ad5-DT:DC-NANPn vectors. To generate Ad5-positive mouse serum, mice were immunised intramuscularly with 1E+8 ifu Ad5(Ovalbumin) (WT hexon) vector (Ad5 E1 and E3 deleted vector with an unmodified hexon expressing ovalbumin instead of GFP under the control of a constitutive CMV promoter encoded at E1 locus) and serum was harvested two-weeks post-immunisation. The results of the neutralization assay demonstrated that IC₅₀ of the neutralizing anti-serum is increased with Ad5-DT:NANP9 and Ad5-DT:NANP18 compared to Ad5-DT alone (FIG. 8C).

### Example 9—Human Coagulation Factor X-Dependent Vector Transduction of SKOV3 Cells

It has been demonstrated that, particularly after intravenous administration, some serotypes of adenovirus including Ad5 can transduce hepatocytes via an interaction between human coagulation Factor X (FX) and the hexon protein of the virus [Simon N. Waddington, John H. McVey, David Bhella, Menzo J. E. Havenga, Stuart A. Nicklin, Andrew H. Baker, Adenovirus Serotype 5 Hexon Mediates Liver Gene Transfer. Cell, 2008, Volume 132, Issue 3, P397-409]. In vitro, FX mediated infection can be assessed by measuring vector transduction of SKOV3 cells, a human ovarian cancer cell line, in the presence or absence of FX.

Ad5(GFP)-DogTag (Ad5) or Ad5(GFP)-DogTag:DogCatcher-NANP18 (Ad5-NANP18) vectors (2E+9 viral particles) were incubated in the presence or absence of human coagulation Factor X (8 μg/mL) on SKOV3 cells for 2 h at 37° C. in serum-free media. Then media was replaced with fresh complete media and plates were incubated for a further 48 h. Infectious titres were then calculated by enumeration of GFP-positive foci by fluorescence microscopy.

The data indicate that coupling of DogCatcher-NANP18 to the Ad5 hexon reduces FX dependent infection of SKOV3 cells, presumably by shielding the viral capsid from interaction with FX.

### Example 10—Mouse Immunogenicity Experiment to Assess CD8⁺ T Cell Responses to Ad5 Surface-Displayed Peptide Antigens

C57BL/6 mice (4/group) were immunised intramuscularly with either surface-display vector Ad5(GFP) HVR5-DT:SnJr-GGSSIINFEKL (5E+9 viral particles, dialysed to remove excess peptide and Snoopligase) (Group 1), the same SIINFEKL surface display vector as in Group 1 but without dialysis to remove excess peptide and Snoopligase (5E+9 viral particles, excess SIINFEKL peptide 0.7 μg) (Group 2), Ad5(GFP) vector (5E+9 viral particles) co-administered with SnJr-GGSSIINFEKL peptide (0.7 μg, not coupled to the vector) (Group 3), or SnJr-GGSSIINFEKL peptide (5 μg, co-administered with 10 μg poly I:C) (group 4) (FIG. 10A). The effective dose of SIINFEKL peptide displayed on the coupled vector from Group 1 was calculated to be approx. 10 ng, taking into account the molecular weight of SnJr-GGSSIINFEKL, a coupling efficiency of 80% hexon coverage (see FIG. 6C, PEP1), 720 copies of hexon per virion, and 5E+9 virions per vaccine dose. The dose of peptide in Group 4 is 500-fold higher than in Group 1. After 14 days post-immunisation, mice were sacrificed and CD8+T cell responses in the spleen were measured by overnight ex vivo IFNγ-ELISPOT.

Spleen ex vivo IFNγ-ELISPOT responses to SIINFEKL, a murine CD8+T cell epitope are shown in FIG. 10B. SIINFEKL responses in Group 1 and Group 2 were higher than those in Group 3, demonstrating that attachment of SnJr-GGSSIINFEKL to the vector capsid is required to achieve potent SIINFEKL T cell responses. Spleen ex vivo IFNγ-ELISPOT responses to DTLVNRIEL (EGFP₁₁₈₋₁₂₆), a murine CD8⁺ T cell epitope present in the encoded GFP transgene expressed by the vector upon cell transduction, are shown in FIG. 10C. There were no significant differences in EGFP₁₁₈₋₁₂₆ responses between Groups 1-3, demonstrating that surface decoration of the vector capsid did not impair T cell responses to the encoded GFP transgene antigen.

### Example 11—Modular Covalent Decoration of the Adenovirus Capsid Via Fusion of SpyCatcher at the C-Terminus of pIX

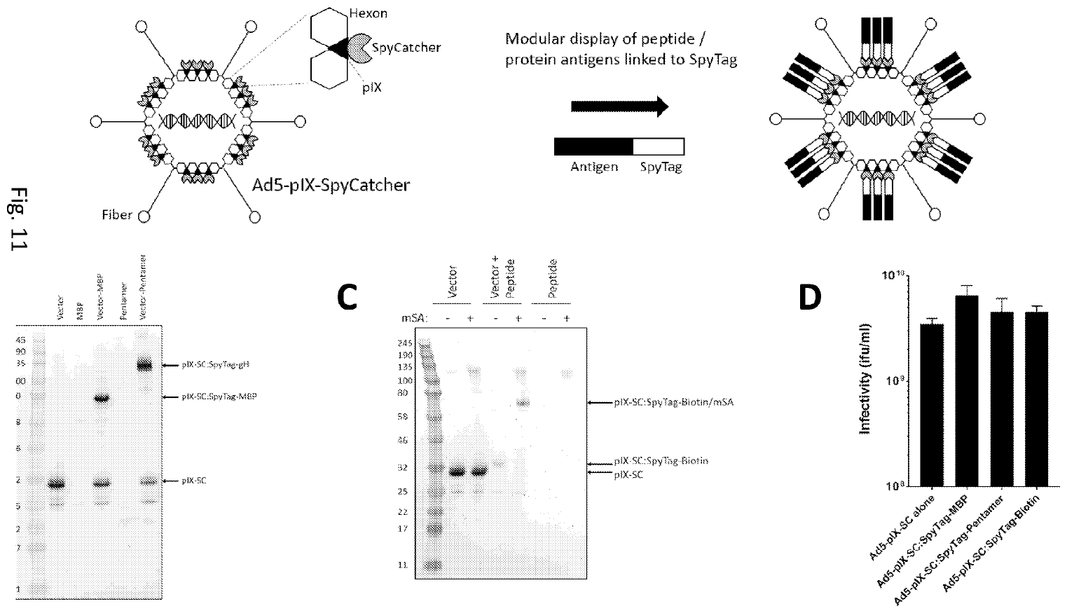

An (EAAAK)₃ linker was inserted between the C-terminus of viral pIX ad the N-terminus of SpyCatcherΔN1 to facilitate successful rescue of the virus and efficient capsid display as shown in FIG. 11A.

Ad5(GFP) pIX-SpyCatcher vectors (3E+9 viral particles) were incubated with SpyTag-Maltose binding protein (SpyTag-MBP, 5 μM) or SpyTag-Human cytomegalovirus pentamer (SpyTag-Pentamer, 2.5 μM) for 16 h at 4° C. (FIG. 11B). Samples containing SpyTag-MBP or SpyTag-Pentamer alone were included as controls. After conjugation, samples were run on SDS-PAGE and transferred to nitrocellulose for Western blotting. pIX-SpyCatcher, pIX-SpyCatcher:SpyTag-MBP, and pIX-SpyCatcher:SpyTag-gH (from the HCMV Pentamer) species were detected using antisera from mice immunised with SpyCatcher protein (FIG. 11B).

Ad5(GFP) pIX-SpyCatcher vectors (3E+9 viral particles) were incubated with biotinylated SpyTag peptide (5 μM) for 16 h at 4° C. (FIG. 11C). To assess peptide coupling efficiency to pIX-SpyCatcher by gel-shift assay, vector material was denatured and boiled in sample buffer including sodium dodecyl sulfate (SDS) and DTT, before cooling and incubating with monovalent streptavidin (mSA) as described in FIG. 6D. pIX-SpyCatcher and pIX-SpyCatcher:SpyTag-biotin/mSA species were detected by SDS-PAGE and Western blotting with anti-SpyCatcher antisera as described in B.

A vector infectivity assay (by GFP focus enumeration, described previously) was performed on the same samples shown in FIGS. 11B and 11C (FIG. 11D). The results demonstrated that Ad5(GFP) pIX-SpyCatcher infectivity is not affected by surface display of SpyTag-protein fusions or peptides.

### Example 12—Viability, Genetic Stability, and Yield of Different Recombinant Adenovirus Vectors with SpyCatcher, SnoopCatcher or DogCatcher Fused to the C-Terminus of pIX

Further research demonstrated that the vector construct described in Example 11 (SpyCatcher fused to the C-terminus of pIX via an EAAAK3 linker) while reactive, was not genetically stable. A range of alternative constructs with alternative linker sequences (GSGGSGGSG (GSG3), GGSEAAAKEAAAKEAAAKGS (EAAAK3), or GGSPANLKALEAQKQKEQRQAAEELANAKKLKEQLEKGS (a helical sequence from ribosomal protein L9, Veggiani et al, Proc Natl Acad Sci USA. 2016 Feb. 2; 113(5):1202-7. doi: 10.1073/pnas.1519214113) (HELIX)), GS hinge sequences either side of the linker, or alternative Catchers (SpyCatcher dN1, SpyCatcher dN1dC2, SnoopCatcher, DogCatcher) were constructed (FIG. 12A). Each vector DNA construct was linearised by PacI restriction digest to release the viral inverted terminal repeats (ITRs) prior to transfection into HEK293A cells. Viability of each construct was assessed by inspecting cell monolayers for development of cytopathic effect (CPE) which would indicate successful rescue of the recombinant virus. Viable viral vector preparations were passaged to increase virus titer, with three passages typically required to obtain sufficient material for purification by CsCl gradient ultracentrifugation (from initial transfection). A forth passage was required to obtain a second batch of each viable vector, and genetic stability was assessed after the third and fourth passage by sequencing of the pIX locus. To assess vector yield, viable vectors were amplified from a total of 10×T150 flasks (total surface area 1500 cm² HEK293A cells), purified by CsCl ultracentrifugation, and infectious titer calculated by GFP infectivity assay as described previously (FIG. 12B).

### Example 13—Reactivity of SnoopCatcher and DogCatcher Fused to the C-Terminus of pIX

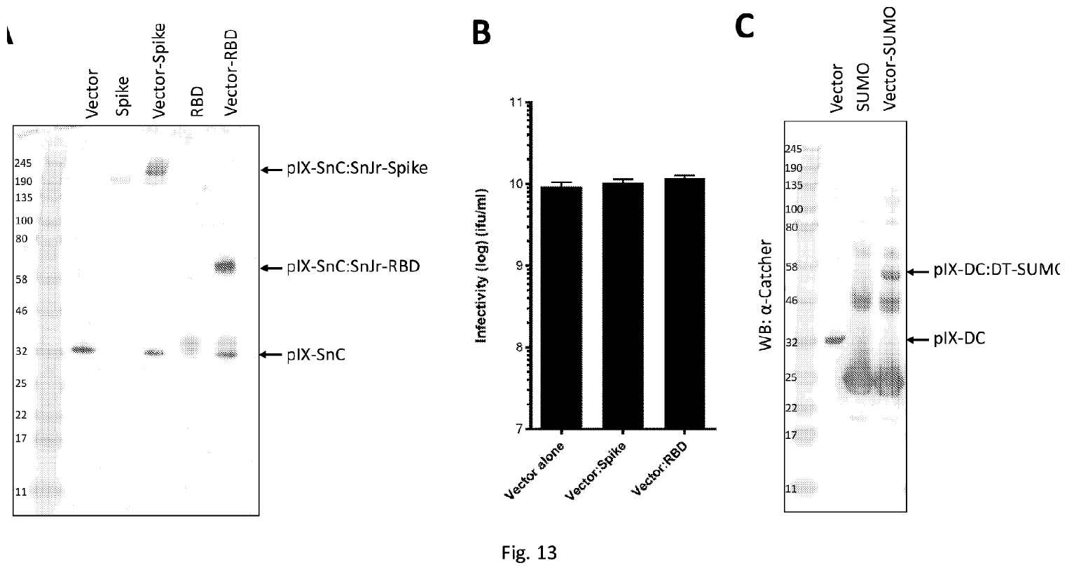

To assess reactivity of Ad5(GFP) pIX-SnoopCatcher with SnoopTagJr-fused ligands, Ad5(GFP) pIX-SnoopCatcher vectors were incubated with tagged full-length SARS CoV2 Spike (Spike) or SARS CoV2 spike receptor binding domain (RBD) proteins. Both recombinant proteins were produced in mammalian suspension 293F cells, with SnoopTagJr fused at the C-terminus. After a 16 h coupling reaction at 4° C., samples were run on SDS-PAGE and a western blot performed using mouse sera with reactivity against SnoopCatcher (sera from mice immunised with IMX313-DogTag:SnoopTag-CIDR:SnoopLigase, Andersson, A. C., Buldun, C. M., Pattinson, D. J. et al. SnoopLigase peptide-peptide conjugation enables modular vaccine assembly. Sci Rep 9, 4625 (2019)) (FIG. 13A). Controls included vector alone (Vector), Spike alone (Spike) and RBD alone (RBD). Species including pIX-SnoopCatcher (pIX-SnC), and covalently coupled pIX-SnoopCatcher:SnoopTagJr-Spike (pIX-SnC:SnJr-Spike) and pIX-SnoopCatcher:SnoopTagJr-RBD (pIX-SnC:SnJr-RBD) are indicated. Immediately after the coupling reactions described in FIG. 13A, a vector infectivity assay (GFP foci) was undertaken with the same samples (FIG. 13B). The data indicate that SnoopCatcher is reactive when fused to the C-terminus of pIX, and that coupling of ligands to pIX does not impair vector infectivity in vitro (despite the size of the SARS CoV2 Spike trimer exceeding 500 kDa).

To assess reactivity of Ad5(GFP) pIX-DogCatcher with DogTag-fused ligands, Ad5(GFP) pIX-DogCatcher vectors were incubated with E coli expressed small ubiquitin modifier (SUMO) protein with DogTag fused at the C-terminus. After a 16 h coupling reaction at 4° C., samples were run on SDS-PAGE and a western blot performed using mouse sera with reactivity against DogCatcher (FIG. 13C). The same mouse sera used for the western blot described in FIG. 13A was also used in FIG. 13C, since DogCatcher shares a significant degree of sequence identity with SnoopCatcher. Controls included vector alone (Vector) and SUMO alone (SUMO). Species pIX-DogCatcher (pIX-DC) and pIX-DogCatcher:DogTag-SUMO (pIX-DC:DT-SUMO) are indicated.

### Example 14—Reactivity of SnoopTagJr and SpyTag Fused to the C-Terminus of pIX

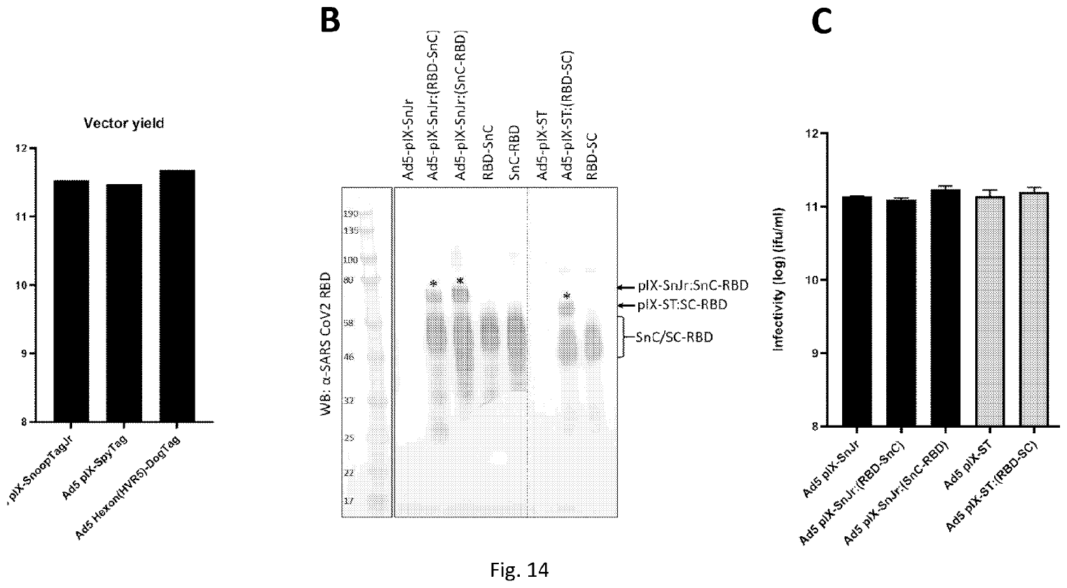

Ad5(GFP) vectors were constructed with SnoopTagJr or SpyTag fused to the C-terminus of pIX, with an EAAAK3 linker between pIX and tag flanked by GGS and GS hinges, similar to the SnoopCatcher and DogCatcher fusion constructs shown in FIG. 12A. Both Ad5 pIX-SnoopTagJr and Ad5 pIX-SpyTag vectors were transfected into HEK293A cells, passaged, and amplified into 10×T150 cm² flasks. Infectious yield of both vectors was high, and comparable to Ad5(GFP) HVR5-DogTag (FIG. 14A).

To assess reactivity of SnoopTagJr and SpyTag displayed as a pIX fusion, Ad5 pIX-SnoopTagJr (Ad5 pIX-SnJr) or Ad5 pIX-SpyTag (Ad5 pIX-ST) vectors were co-incubated with recombinant mammalian (Chinese Hamster Ovary, CHO) cell expressed SARS CoV2 RBD proteins fused to either SnoopCatcher (RBD-SnC or SnC-RBD, C- or N-terminal fusions to RBD respectively) or SpyCatcher (RBD-SC, C-terminal fusion). After a 16 h coupling reaction at 4° C., samples were run on SDS-PAGE and a western blot performed using an anti-SARS CoV2 RBD polyclonal antibody (Sino Biological) (FIG. 14B). Protein species representing coupling between Tag and Catcher modules are indicated (*). Immediately after the coupling reactions described in FIG. 14B, a vector infectivity assay (GFP foci) was undertaken with the same samples (FIG. 14C). Coupling of ligands did not impair infectivity of either Ad5 pIX-SnoopTagJr or Ad5 pIX-SpyTag.

1. An adenoviral vector for preparation of a prophylactic or therapeutic composition, comprising at least one modification in a capsid protein, wherein said modification comprises the inclusion of a first peptide partner in a capsid protein, and said first peptide is capable of forming a covalent bond with a second peptide partner.

2. An adenoviral vector according to claim 1 where said second peptide partner is attached to an entity, optionally an antigen, a targeting moiety or a shielding entity.

3. An adenoviral vector according to claim 1 or claim 2 wherein at least one modification in a capsid protein is in a major capsid protein or in a minor capsid protein.

4. An adenoviral vector according to any one of claims 1 to 3 wherein at least one modification in a major capsid protein is in the hexon protein, optionally in a HVR loop.

5. An adenoviral vector according to any one of claims 1 to 3 wherein at least one modification in a minor capsid protein is in the pIX protein.

6. An adenoviral vector according to any one of claims 1 to 5 wherein at least one modification to a capsid protein is the insertion or fusion of said first peptide partner to the capsid protein.

7. An adenoviral vector according to any one of claims 1 to 6 wherein said covalent bond is an isopeptide.

8. An adenoviral vector according to claim 4 wherein the first peptide partner is DogTag and wherein the second peptide partner is DogCatcher or SnoopTagJr or SnoopTag.

9. An adenoviral vector according to claim 5 wherein a) a first peptide partner is SpyCatcher, optionally wherein the second peptide partner is SpyTag;
b) a first peptide partner is SnoopCatcher, optionally wherein the second peptide partner is SnoopTagJr or SnoopTag;
c) a first peptide partner is DogCatcher, optionally wherein the second peptide partner is DogTag;
c) a first peptide partner is SnoopTagJr, optionally wherein the second peptide partner is SnoopCatcher; or
d) a first peptide partner is SpyTag, optionally wherein the second peptide partner is SpyCatcher.

10. An adenoviral vector according to any one of claims 2 to 9 wherein the second peptide is attached to an antigen via genetic fusion or chemically attached to an antigen.

11. An adenoviral vector according to claim 10 wherein said antigen is a tumour-associated antigen, such as a neoepitope or neoantigen, a self-antigen, or an antigen from a pathogen, such as a virus, bacterium, parasite or fungus, optionally wherein the virus is SARS-CoV-2.

12. An adenoviral vector according to any preceding claim wherein the size of said second peptide partner attached to an entity is over 15 kDa, 20 kDa, 30 kDa, 40 kDa, 50 kDa, 60 kDa, 70 kDa, 80 kDa, 90 kDa or 100 kDa in size.

13. An adenoviral vector according to claim 12 wherein said second peptide partner attached to an entity shields the adenovirus from antibody binding to the capsid.

14. An adenoviral vector according to any preceding claim for use in the manufacture of a vaccine comprising the addition of a second peptide partner attached to an antigen to said adenoviral vector.

15. A vaccine comprising the adenoviral vector of any preceding claim, wherein an antigen is attached to the adenoviral vector via the second peptide partner.

16. A vaccine as claimed in claim 15 wherein said antigen comprises at least one antigen specifically expressed on a tumour or cancer cell, or virus, optionally SARS-Cov-2.

17. A method of producing an adenoviral vector as claimed in any one of claims 1 to 14 comprising:
introducing a nucleic acid which encodes a first peptide partner into the nucleic acid encoding a capsid protein;
inserting the modified capsid gene into the genome of an appropriate adenovirus; and
infecting a cell with said adenovirus and collecting the released progeny virus.

18. A vaccine comprising an adenoviral vector as claimed in claim 15 or 16 and an attached antigen for use in the treatment or prevention of a disease.

19. A kit comprising an adenoviral vector as claimed in any one of claims 1 to 14.

20. A vaccine comprising an adenoviral vector as claimed in claim 1, wherein
a. said first peptide partner is SpyCatcher which is fused to the pIX capsid protein, and said second peptide partner is SpyTag, which is attached to an antigen, and wherein SpyCatcher and SpyTag are bound covalently through an isopeptide bond;
b. said first peptide partner is SnoopCatcher which is fused to the pIX capsid protein, and said second peptide partner is SnoopTagJr, or SnoopTag which is attached to an antigen, and wherein SnoopCatcher and SnoopTagJr or SnoopTag are bound covalently through an isopeptide bond; or
c. said first peptide partner is DogCatcher which is fused to the pIX capsid protein, and said second peptide partner is DogTag which is attached to an antigen, and wherein DogCatcher and DogTag are bound covalently through an isopeptide bond; or
d. said first peptide partner is SnoopTagJr which is fused to the pIX capsid protein, and said second peptide partner is SnoopCatcher, which is attached to an antigen, and wherein SnoopTagJr and SnoopCatcher are bound covalently through an isopeptide bond;
e. said first peptide partner is SpyTag which is fused to the pIX capsid protein, and said second peptide partner is SpyCatcher, which is attached to an antigen, and wherein SpyTag and SpyCatcher are bound covalently through an isopeptide bond.

21. A vaccine comprising an adenoviral vector as claimed in claim 1, wherein said first peptide partner is DogTag which is inserted into HVR loops of the hexon protein, and said second peptide partner is DogCatcher or SnoopTagJr, said second peptide partner being attached to an antigen, and wherein DogTag and DogCatcher/SnoopTagJr are bound covalently through an isopeptide bond.

22. An oncolytic virus preparation comprising an adenoviral vector as claimed in claim 1, wherein said second peptide partner is attached to a shielding entity or a targeting moiety.

23. An adenoviral vector as claimed in any one of claims 1 to 14 wherein said adenovirus is any serotype or species, optionally a human adenovirus.

24. An adenoviral vector as claimed in any one of claims 1 to 14 wherein said adenovirus is replication-competent, optionally modified to replicate only in selective cells.

25. An adenoviral vector as claimed in any one of claims 1 to 14 wherein said adenovirus is replication-incompetent.

26. An adenoviral vector as claimed in any one of claims 1 to 14 wherein said adenovirus is genetically modified to encode a transgene and optionally surface decorated via an isopeptide linkage.

27. An adenoviral vector capable of surface decoration via an isopeptide linkage via a modified capsid protein.
